(19) United States
(12) Patent Application Publication
(10) Pub. No.: US 2026/0305550 A1
(43) Pub. Date: Oct. 8, 2026

(54) GENETIC DETERMINANTS CONFERRING IMPROVED TOBRFV RESISTANCE

(71) Applicant: VILMORIN & CIE, Paris (FR)

(72) Inventors: Stéphanie MALLARD, Ledenon (FR); Lilian FONTANET, Portes-lès-Valence (FR); Shai KOUSSEVITZKY, Tel Aviv (IL); Nicolas SOUNAC, Saint-Remy de Provence (FR); Yaniv ROTEM, Tel Aviv (IL); Thomas DUGE DE BERNONVILLE, Loire-Authon (FR)

(73) Assignee: VILMORIN & CIE, Paris (FR)

(21) Appl. No.: 19/479,065

(22) PCT Filed: Apr. 26, 2024
(86) PCT No.: PCT/EP2024/061670

§ 371 Date: Oct. 27, 2025

(30) Foreign Application Priority Data
Apr. 28, 2023 (EP) 23170875.1

(51) Int. Cl. A01H 1/00; A01H 1/04; C12Q 1/6895

(52) U.S. Cl. CPC A01H 1/126; A01H 1/045; C12Q 1/6895; C12Q 2600/13; C12Q 2600/156

(57) ABSTRACT

A combination of genetic determinants in seeds and cells provide improved ToBRFV resistance and to different methods and use, wherein a tomato plant is resistant to Tomato Brown Rugose Fruit virus (ToBRFV). Included in its genome are the combination of a resistance quantitative trait locus (QTL) on chromosome 8 introgressed from S. habrochaites and conferring resistance to ToBRFV and at least one QTL on chromosome 11, conferring to the plant foliar resistance to ToBRFV when present homozygously, or the Tm-1 gene, the plant having an improved resistance to ToBRFV.

The present invention relates to resistance in plants of Solanum lycopersicum, also known as Lycopersicum esculentum, to the tobamovirus Tomato Brown Rugose Fruit virus (ToBRFV, previously also known as TBRFV).

More specifically, the present invention relates to tomato plants and fruits comprising one or more genetic determinants, that lead to resistance to the Tomato Brown Rugose Fruit virus, especially the combination of two genetic determinants giving rise to an improved resistance, in terms of inter alia absence of viral replication, duration of the resistance and/or acceptable temperature for the resistance. The invention further relates to the genetic determinants, especially a new ToBRFV resistance QTL, and to markers linked to these one or more genetic determinant(s), for identification and/or selection of plants carrying such resistance. The invention also relates to the seeds and progeny of such plants and to propagation material for obtaining such plants, and to different uses of these plants.

### BACKGROUND OF THE INVENTION

All cultivated and commercial forms of tomato belong to a species most frequently referred to as Lycopersicon esculentum Miller. Lycopersicon is a relatively small genus within the extremely large and diverse family Solanaceae which is considered to consist of around 90 genera, including potato, pepper, tobacco and eggplant. The genus Lycopersicon has been divided into two subgenera, the esculentum complex which contains those species that can easily be crossed with the commercial tomato and the peruvianum complex which contains those species which are crossed with considerable difficulty (Stevens, M., and Rick, C. M. 1986). Due to its value as a crop, L. esculentum Miller has become widely disseminated all over the world. Even if the precise origin of the cultivated tomato is still somewhat unclear, it seems to come from the Americas, being native to Ecuador, Peru and the Galapagos Island and initially cultivated by Aztecs and Incas as early as 700 AD. Mexico appears to have been the site of domestication and the source of the earliest introduction. It is supposed that the cherry tomato, L. esculentum var. cerasiforme, is the direct ancestor of modern cultivated forms.

Tomato is grown for its fruit, widely used as a fresh market or processed product. As a crop, tomato is grown commercially wherever environmental conditions permit the production of an economically viable yield. Most fresh market tomatoes are harvested by hand at vine ripe and mature green stage of ripeness. Fresh market tomatoes are available year-round.

Tomato is a normally simple diploid species with twelve pairs of differentiated chromosomes. However, polyploidy tomato is also part of the present invention. The cultivated tomato is self-fertile and almost exclusively self-pollinating. The tomato flowers are hermaphrodites. Commercial cultivars were initially open pollinated. As hybrid vigor has been identified in tomatoes, hybrids are replacing the open pollinated varieties by gaining more and more popularity amongst farmers with better yield and uniformity of plant characteristics. Due to its wide dissemination and high value, tomato has been intensively bred. This explains why such a wide array of tomato is now available. The shape may range from small to large, and there are cherry, plum, pear, blocky, round, and beefsteak types.

A variety of pathogens affect the productivity of tomato plants, including viruses, fungi, bacteria and nematodes, as well as arthropod pests-insects and mites. Tomatoes are inter alia susceptible to many viruses and virus resistance is therefore of major agricultural importance.

Tobamoviruses are among the most important plant viruses causing severe damages in agriculture, especially to vegetable and ornamental crops around the world. Tobamoviruses are easily transmitted by mechanical means while there is no evidence of a natural vector, as well as through seed transmission. Tobamoviruses are generally characterized by a rod-shaped particle of about 300 nm, their structure consists in a single stranded, positive RNA genome encoding four proteins, encapsidated by 17 KDa coat protein (CP) molecules.

In tomatoes, tobacco mosaic virus (TMV), tomato mosaic virus (ToMV) may cause severe damage to crop production, by weakening of the plants and through irregular ripening (fruits having yellowish patches on the surface and brownish spots beneath the surface). Several resistance genes against TMV and ToMV have been identified by plants breeders over the years.

The first resistance gene identified was the Tm-1 gene, conferring resistance to TMV. This gene, introgressed from S. habrochaites, is incompletely dominant and homozygosis was generally required for the TMV resistance. The Tm-1 gene was however overcome within about one year of introduction to commercial horticulture, rendering the pursuing of its introduction into other commercial lines entirely useless (Pelham et al, 1970). The vast majority of the circulating TMV and ToMV strains, infecting the commercial plants, are indeed resistant to plants harboring the Tm-1 gene, such that this gene is no longer considered as a resistance gene against TMV/TOMV infection in commercial plants. The use of this Tm-1 gene has now been almost completely abandoned in favor of alternative resistance genes.

For the last decades, all modern indeterminate tomato varieties and many of the determinate tomato varieties indeed contain the Tm-2 gene or preferably the Tm-22 allele of this gene (also noted Tm2.2 or Tm-2(2)), which give them immunity to almost all known races of Tobamoviruses which affected commercial tomatoes (ToMV and TMV) before 2014. The resistance gene Tm2.2 appears to also mostly confer resistance to ToMMV (Nagai et al, 2019; Sui et al, 2017).

During 2014-2015, a most severe outbreak of a new Tobamovirus affected all the tomato production areas in Israel and in Jordan, and all the varieties that were grown, which are all ToMV-resistant varieties, were severely affected by a new Tobamovirus, showing typical TMV/ToMV-like symptoms. In addition to symptoms on leaves, which are quite like those which are produced by TMV/ToMV, the new virus also caused severe fruit symptoms, which are much more frequent and severe than the known occurrence of fruit symptoms produced by TMV/TOMV.

Both Jordanian and Israeli isolates of the virus were fully sequenced (Luria et al, 2017). The virus was named “Tomato Brown Rugose Fruit Virus-ToBRFV” (Salem et al. 2016).

ToBRFV spread rapidly, and now it is found in most of the Mediterranean and European countries, as well as in America and East Asia, thus considered today as a global major disease of tomatoes.

Generally, ToBRFV is causing significant damage to the tomato leaves, and significant damage to the tomato fruit quality—particularly in large fruit varieties. It causes dramatic weakening of the plants which limits the growth season length. For example, in Israel-until the outbreak of ToBRFV in 2014, the typical main crop season started in August transplanting and ended in May or June—a season of 10-11 months. Since the ToBRFV outbreak, the growing season is much shorter—not more than 4 months, and the growers shifted to a situation of two short seasons instead of 1 long season per year, thus giving rise to a diminished yield for increased costs.

Identification of tomato plants which display resistance to ToBRFV and localization and identification of genetic determinants, also referred to hereafter as QTLs (Quantitative Trait Locus) that lead to tolerance to the Tomato Brown Rugose Fruit virus have recently been described in WO2018/219941. Two QTLs, designated “QTL1” and “QTL2”, on chromosome 6 and 9 respectively, confer independently or in combination an improved tolerance or resistance in the fruits of a tomato plant infected or likely to be infected by the ToBRFV, when present homozygously in a S. lycopersicum background. A third QTL, “QTL3”, on chromosome 11, confers an improved tolerance or resistance in the leaves of a tomato plant infected or likely to be infected by the ToBRFV, when present homozygously in a S. lycopersicum background.

Whereas these QTLs, either alone or in combination, provide tolerance or resistance to ToBRFV, this tolerance/resistance appears quantitative, and plants are not free of virus, thus propagating the virus to further plants. Moreover, these QTLs are described as providing resistance only when present homozygously.

In document WO2020/249798, it was further disclosed that the tolerance provided by the already identified QTLs “QTL1”, “QTL2” and “QTL3” could be improved by addition of the Tm-1 gene.

WO2019/110130 and WO2019/110821 disclose the identification of 3 different QTLs on chromosomes 6, 11 and 12, introgressed from S. pimpinellifolium and allegedly conferring resistance or tolerance to ToBRFV.

WO2020/018783 discloses a genetic region on tomato chromosome 11 that comprises a Stemphylium resistance allele from S. pimpinellifolium, and allegedly also comprises an associated ToBRFV resistance allele. As both resistance alleles are linked to the same markers according to the disclosure of this document, introgression of only ToBRFV resistance is not made possible, especially in plants already resistant to Stemphylium but susceptible to ToBRFV.

A further source of resistance was identified in a wild S. pimpinellifolium plant and is disclosed in WO2021/245282. This resistance is imparted by two distinct QTLs on chromosome 9 and 11, wherein the QTL on chromosome 9 (named “QTL9”) imparts resistance at the fruit and leaf level, and the QTL on chromosome 11 (named “QTL11”) essentially at the level of the leaves. Presence of viral sequences is also greatly diminished or even abrogated in plants comprising said QTLs, shortly after inoculation.

WO2020/148021 describes a resistance gene, on chromosome 8, allegedly conferring resistance to ToBRFV; this gene codes for an NBS-LRR protein (Nucleotide-binding site Leucine-rich repeat). Viral replication seems however to still take place in infected plants. Moreover, this resistance is generally broken at high temperatures. Finally, there are doubts that the absence of symptoms will last until mature fruits are obtained, especially in case of early infection.

### SUMMARY

The present inventors have identified a resistance against ToBRFV in a wild S. habrochaites plant and have been able to introgress this resistance into S. lycopersicum plants, thus obtaining S. lycopersicum tomato plants resistant to ToBRFV. The resistance to ToBRFV is imparted by the introgressed sequences from S. habrochaites, namely a quantitative trait locus (QTL) on chromosome 8, transferable to different S. lycopersicum genetic backgrounds.

This QTL on chromosome 8, referred to as QTL8 in the following, confers a high level of resistance to the Tomato Brown Rugose Fruit virus (ToBRFV), especially the QTL as present in the genotype of the deposited seeds LVSTBRFVRES3, NCIMB 44125. The inventors have moreover been able to identify a consensus sequence of different versions of a repeated gene within this QTL8 providing the resistance, referred to as resistance gene in the following, or resistance gene on chromosome 8.

Moreover, the inventors have demonstrated that the combination of this QTL8 of the invention with a QTL on chromosome 11, also providing resistance to ToBRFV, for example “QTL11” as disclosed in WO 2021/245282 or “QTL3” as disclosed in WO2018/219941, unexpectedly confers an improved or enhanced ToBRFV resistance. The combination of this QTL8 with Tm-1 gene, in S. lycopersicum genome also provides an improved ToBRFV resistance.

The present invention thus provides S. lycopersicum plants that display an improved resistance to ToBRFV, including commercial plants, lines and hybrids, as well as methods that produce or identify S. lycopersicum plants or populations (germplasm) that display improved resistance to ToBRFV. The present invention also discloses molecular genetic markers, especially Single Nucleotide Polymorphisms (SNPs), linked to the QTL and/or genes of the invention responsible for enhanced resistance to ToBRFV. Plants obtained through the methods and uses of such molecular markers are also provided.

Said resistance is moreover easily transferable to different genetic backgrounds, i.e. into various tomatoes, and the invention also extends to different methods allowing the transfer or introgression of the QTLs and/or gene conferring the phenotype.

The invention also provides several methods and uses of the information linked to these SNPs associated to the QTL or gene conferring the ToBRFV resistance, inter alia methods for identifying ToBRFV resistant plants, as well as methods for improving the yield of tomato production in an environment infested by ToBRFV and methods for protecting a tomato field from ToBRFV infestation.

The present invention also provides these introgressed sequences, also named here QTLs, or resistance gene, conferring the phenotype of ToBRFV resistance and/or improved ToBRFV resistance, at the level of the tomato leaves and/or fruits of the tomato plants infected by the ToBRFV.

### Definitions

The term “Resistance” is as defined by the ISF (International Seed Federation) Vegetable and Ornamental Crops Section for describing the reaction of plants to pests or pathogens, and abiotic stresses for the Vegetable Seed Industry. Specifically, by resistance, it is meant the ability of a plant variety to restrict the growth and development of a specified pest or pathogen and/or the damage they cause when compared to susceptible plant varieties under similar environmental conditions and pest or pathogen pressure. Resistant varieties or plants may exhibit some disease symptoms or damage under heavy pest or pathogen pressure. Two levels of resistance are defined:

High Resistance (HR): plants that highly restrict the growth and/or development of the specified pest and/or the damage it causes under normal pest pressure when compared to susceptible plants. These plants may, however, exhibit some symptoms or damage under heavy pest pressure.

Intermediate Resistance (IR): plants that highly restrict the growth and/or development of the specified pest and/or the damage it causes but may exhibit a greater range of symptoms or damage compared to high resistance plants. Intermediate resistant plants will still show less severe symptoms or damage than susceptible plants when grown under similar environmental conditions and/or pest pressure.

The term “Tolerance” is normally used to describe the ability of a plant to endure abiotic stresses without serious consequences for growth, appearance and yield.

In the literature and patents, this term is however also used to indicate a phenotype of a plant wherein at least some of the disease-symptoms remain absent upon exposure of said plant to an infective dose of virus, whereby the presence of a systemic or local infection, virus multiplication, at least the presence of viral genomic sequences in cells of said plant and/or genomic integration thereof can be established, at least under some culture conditions. Tolerant plants are therefore resistant for symptom expression but symptomless carriers of the virus. Sometimes, viral sequences may be present or even multiply in plants without causing disease symptoms. It is to be understood that a tolerant plant, although it is infected by the virus, is generally able to restrict at least moderately the growth and development of the virus.

For this reason, tolerant plants according to this definition are best characterized by Intermediate Resistant plants.

In case of ToBRFV, by leave resistance, or foliar resistance, it is meant the phenotype of a plant wherein the disease symptoms on the leaves remain absent, or are less important, upon exposure of said plant to an infective dose of ToBRFV. Disease symptoms on the fruits may however be present on infected plants.

By fruit resistance, in case of ToBRFV, it is meant the phenotype of a plant wherein the disease symptoms on the fruits remain absent, or are less important, upon exposure of said plant to an infective dose of ToBRFV. Disease symptoms on the leaves may however be present on infected plants.

Symptoms on leaves of ToBRFV infection generally include mosaic, distortion of the leaflets and in many cases also shoestrings like symptoms. Symptoms on fruits of ToBRFV infection generally include typical yellow lesions (discoloration) and deformation of the fruits. In many cases there are also “chocolate spots” on the fruits.

Susceptibility: The inability of a plant to restrict the growth and development of a specified pest or pathogen; a susceptible plant displays the detrimental symptoms linked to the virus infection, namely the foliar damages and fruit damages in case of ToBRFV infection.

A S. lycopersicum plant susceptible to Tomato Brown Rugose Fruit virus, is for example the commercially available variety Candela as mentioned in the 2015 Salem et al. publication or the commercially available variety Mobaci or Momor.

All commercially available varieties of tomato grown in ToBRFV infected area are, to date, i.e. before the present invention, susceptible to ToBRFV, or less resistant than the claimed plants, for those plants bearing tolerance QTLs, such as the deposited seeds of HAZTBRFVRES1, mentioned in the PCT application WO2018/219941 or those bearing resistance QTLs, such as the deposited seeds of LVSTBRFVRES2, mentioned in the PCT application WO2021/245282.

A plant according to the invention has thus at least improved resistance or tolerance to ToBRFV, with respect to the variety Candela, and more generally with respect to any commercial variety of tomato grown in ToBRFV infected area, including tolerant plants, and with respect to HAZTBRFVRES1 and LVSTBRFVRES2.

The improved resistance with respect to the plants corresponding to HAZTBRFVRES1 is demonstrated in example 3 of the experimental section and FIG. 1.

As used herein, the term “offspring” or “progeny” refers to any plant resulting as progeny from a vegetative or sexual reproduction from one or more parent plants or descendants thereof. For instance, an offspring plant may be obtained by cloning or selfing of a parent plant or by crossing two parental plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids. Progeny thus includes the 1^{st} generation, obtained after one cross, as well as the 2^{nd} generation, obtained from the 1^{st} generation after a further cross. Preferably, a progeny refers to plants obtained from a F1 as defined and one or several backcrosses or selfings, preferably less than 10. The introgressed sequences are thus limited to those transferred from the first crossing.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, “genetic determinant” and/or “QTL” refers to any segment of DNA associated with a biological function. QTLs and/or genetic determinants include, but are not limited to, genes, coding sequences and/or the regulatory sequences required for their expression. They can also include nonexpressed DNA segments that, e.g. form recognition sequences for other proteins.

As used herein, the term “genotype” refers to the genetic makeup of an individual cell, cell culture, tissue, organism (e.g., a plant), or group of organisms.

As used herein, the term “heterozygote” refers to a diploid or polyploid individual cell or plant having different alleles (forms of a given gene, genetic determinant or sequences) present at least at one locus.

As used herein, the term “heterozygous” refers to the presence of different alleles (forms of a given gene, genetic determinant or sequences) at a particular locus.

As used herein, “homologous chromosomes”, or “homologs” (or homologues), refer to a set of one maternal and one paternal chromosomes that pair up with each other during meiosis. These copies have the same type of genes at the same loci and the same centromere location, but they may differ by their sequences or alleles.

As used herein, the term “homozygote” refers to an individual cell or plant having the same alleles at one or more loci on all homologous chromosomes.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “locus” (plural: “loci”) refers to any site that has been defined genetically, this can be a single position (nucleotide) or a chromosomal region. A locus may be a gene, a genetic determinant, a part of a gene, or a DNA sequence, and may be occupied by different sequences. A locus may also be defined by a SNP (Single Nucleotide Polymorphism), by several SNPs, or by two flanking SNPs.

The invention encompasses plants of different ploidy levels, essentially diploid plants, but also triploid plants, tetraploid plants, etc.

### DETAILED DESCRIPTION OF THE INVENTION

The present inventors have identified a resistance QTL which not only provides to S. lycopersicum plants resistance to the Tomato Brown Rugose Fruit virus (ToBRFV), but also confers an improved resistance in the fruits and/or leaves of a tomato plant infected or likely to be infected by the virus, when combined with a tolerance or resistance QTL on chromosome 11.

The seeds and plants according to the invention have been obtained by transfer from a wild plant of S. habrochaites into S. lycopersicum genetic background, of a QTL on chromosome 8, referred to as QTL8, which provides ToBRFV resistance; the inventors have moreover combined this QTL8 with a QTL on chromosome 11, such as the “QTL11” introgressed from S. pimpinellifolium and described in WO2021/245282, or the “QTL3” described in WO2018/219941, and they obtained unexpectedly an improved, increased or enhanced resistance to ToBRFV. Such an improved, increased or enhanced resistance is not the addition of the individual effect of each gene or QTL, and could not have been predicted on the basis of the individual effects of each gene or QTL.

The ToBRFV resistance is improved, increased or enhanced with respect to the resistance provided by QTL8, or by the corresponding resistance gene on chromosome 8, when present alone; the resistance is also improved, increased or enhanced with respect to the resistance conferred by the resistance or tolerance QTL on chromosome 11, when present alone homozygously.

The inventors have also demonstrated that the resistance QTL (QTL8) confers an improved resistance in the fruits and/or leaves of a tomato plant infected or likely to be infected by the virus, when combined with the Tm-1 gene, especially regarding absence or reduced viral replication in infected plants, in addition to absence or reduced symptoms of infection. The resistance is preferably improved, increased or enhanced with respect to plants having only a Tm-1 gene, or only the resistance gene or QTL on chromosome 8.

According to a first aspect, the invention is thus directed to a plant or seed of Solanum lycopersicum resistant to Tomato Brown Rugose Fruit virus (ToBRFV) comprising in its genome or genotype the combination of:
(a) a resistance quantitative trait locus (QTL) on chromosome 8 conferring resistance to ToBRFV, and
(b) at least one quantitative trait locus (QTL) on chromosome 11, conferring to the plant foliar resistance to ToBRFV when present homozygously,
   wherein said plant has an improved resistance with respect to the resistance conferred by the QTL on chromosome 8 or 11 alone.

The combination of the genetic elements or determinants (a) and (b) mentioned above is referred to in the following as the combination of the invention, or the combination of genetic determinants of the invention. The invention is also directed to a cell of such a plant or seed or plant part, comprising the combination of genetic elements conferring the improved resistance.

By improved resistance, it is to be understood that one or more of the following features is improved, enhanced or increased in case of ToBRFV infection, by comparison to the genetic elements taken in isolation:
absence of symptoms, i.e. the infected plants do not show any sign of infection, both on the fruits and on the leaves, even under heavy viral pressure or with very aggressive strains, or less signs than plants without the combination of the invention;
persistence of the phenotypic resistance, i.e. absence of disease symptoms, during a longer period than without the combination of the invention, especially during at least 4 months, preferably at least 5 months, even preferably at least 6 months after infection, or even more than 6 months. In case of early infection, the resistance lasts during a prolonged period extending at least until the mature stage of the plant;
reduction of viral replication; i.e. not only the plant has no disease symptoms, but the viral replication in the plant is reduced with respect to a plant not comprising the combination of the invention, thus limiting or abrogating the capacity to propagate the virus;
range of effective temperatures; i.e. the maximal temperature at which the resistance is lost is superior to the maximal temperature for plants not comprising the combination; or the maximal daytime temperature at which the resistance is lost is superior to the maximal daytime temperature for plants not comprising the combination. Such a maximal temperature or daytime temperature is at least 25° C., preferably at least 28° C., or 30° C. for a plant of the invention. Jewehan at al, 2022, indeed disclose that, in the absence of the combination of the invention, the resistance is broken at elevated temperature, at least at 33° C., but probably even at temperatures above 24° C. or above 25° C.;
scope of resistance; namely the ToBRFV resistance is exhibited irrespective to the ToBRFV strain, or for more strains than for plants not comprising the combination. The resistance is preferably exhibited against all different strains isolated from several geographical areas.

The comparison is to be made with respect to a plant not comprising the combination of genetic determinants of the invention, especially a plant with only one genetic determinant of the invention, but not the second one.

It is to be understood that the combination of genetic determinants of the invention improves or increases at least one of the above features, potentially more than one, for example 2 or 3 of them; according to a preferred embodiment, the resistance is improved or increased with respect to at least 2 of these features. According to an embodiment, the resistance is increased or improved with respect to all these features. According to another embodiment, the enhanced resistance is the combination of an increased resistance especially with respect to leaf symptoms, with an increased reduction of the viral replication, particularly at high temperatures, e.g. day temperatures above 33° C., or above 35° C., or even above 40° C., during at least 2 or 3 consecutive days.

It is pointed out in this respect that whether the resistance is increased with one or the other feature mentioned above may depend on the genetic background of the plant or of the type of ToBRFV strain, insofar as the resistance to ToBRFV is namely the result of the interaction between a specific plant and a specific virus strain. Irrespective of the genetic background of the plant or viral strain, the combination of the genetic determinants of the invention results in an improvement in at least one of the factors mentioned above.

In some aspects also encompassed by the present invention, the increased resistance is obtained by a combination of the genetic determinant (a) mentioned above, with a genetic determinant (b′) corresponding to the Tm-1 gene.

The tolerance/resistance phenotype can be tested and scored as described in the experimental section, by natural infection, or by artificial inoculation at the first leaves level, or at the fruit level. Other protocols are also disclosed in the prior art (see Jewehan et al, 2022).

Presence of viral sequences can be assayed by ELISA or Polymerase Chain Reaction (PCR), especially quantitative PCR (qPCR). Suitable protocols, including suitable primers for the PCR or primers and probe for qPCR are also disclosed in the experimental section of the application and known from the prior art. Viability of virus can be tested by bioassay on tobacco plants.

Regarding the first genetic determinant (a), namely the resistance QTL on chromosome 8, this QTL is generally referred to as the “resistance QTL on chromosome 8” or “resistance QTL of chromosome 8”, or QTL8 in the following. This QTL corresponds to sequences introgressed from S. habrochaites.

Such introgressed sequences are introgressed on chromosome 8, preferably between the positions 59100098 and 61782030, according to genome assembly SL3.0. These introgressed sequences are the S. habrochaites sequences corresponding to the segment 59100098 and 61782030 in the S. lycopersicum genome.

According to another embodiment, the introgressed sequences from S. habrochaites constituting the QTL8 are introgressed between the position corresponding to the SNP marker TO-0039023 and SNP marker TO-0202975. A plant, seed or cell of the invention thus comprises, in its chromosome 8, in the region flanked by these markers, introgressed sequences originating from S. habrochaites. These introgressed sequences are the corresponding S. habrochaites sequences in the same region. The introgressed sequences from S. habrochaites provide resistance to ToBRFV.

The specific polymorphisms corresponding to the SNPs (Single Nucleotide Polymorphism) or markers referred to in this description, as well as the flanking sequences of these SNPs or markers in the S. lycopersicum genome, are given in the experimental section (see inter alia table A) and the accompanying sequence listing. Their location (chromosome and position) with respect to the version 2.40, 2.50 or 3.0 of the tomato genome and their flanking sequences are also illustrated in this table.

It is to be noted in this respect that, by definition, a SNP refers to a single nucleotide in the genome, which is variable depending on the allele which is present, whereas the flanking nucleotides are identical. For ease of clear identification of the position of the different SNPs, their position is given in the tables, by reference to the tomato genome sequence in its version 2.40 or 2.50 and by reference to their flanking sequences, identified by SEQ ID number. In the sequence associated with a specific SNP in the present application, for example SEQ ID NO:38 for the SNP TO-0202974, only one nucleotide within the sequence actually corresponds to the polymorphism, namely the 151^{st} nucleotide of SEQ ID NO:38 corresponds to the polymorphic position of SNP TO-0202974, which can be A or G as indicated in table A. The flanking sequences are given for positioning the SNP in the genome but are not part of the polymorphism as such. The polymorphic nucleotide which is indicative, namely A or G, corresponds to the position 60 612 041 in the tomato genome SL2.50, indicated in the table. Detection of a SNP marker, or of an allele of this SNP therefore refers to the detection of the polymorphic nucleotide of this marker and does not require all the flanking sequences to be identical.

A genomic or chromosomal region identified by flanking sequences, e.g. SNPs markers, is thus defined clearly and non-ambiguously.

A genomic region delimited or flanked by two SNPs X and Y refers to the section of the genome, more specifically of a chromosome, lying between the positions of these two SNPs and preferably comprising said SNPs, therefore the nucleotide sequence of this chromosomal region begins with the nucleotide corresponding to SNP X and ends with the nucleotide corresponding to SNP Y, i.e. the SNPs are comprised within the region they delimit, according to the invention.

By “introgressed sequences from S. habrochaites” present at a given locus of the genome of a S. lycopersicum plant, cell or seed, it is to be understood that the genomic sequences found at this locus have the same sequence as the corresponding genomic sequences found in the S. habrochaites donor, i.e. in the wild introgression partner, at the same locus.

By “introgressed sequences from S. habrochaites present within a given region delimited or flanked by two SNPs X and Y” of the genome of a S. lycopersicum plant, cell or seed, it is to be understood that all or part of the genomic sequence lying between the positions of these two SNPs, are introgressed sequences from S. habrochaites.

The presence of introgressed sequences from S. habrochaites into the genome of a S. lycopersicum plant, seed or cell may for example be shown by GISH (genetic in situ hybridization). GISH is indeed a powerful technique for detection of the introgression of chromatin material from one species or subspecies onto another species. The advantage of GISH is that the introgression process is visualized by means of “pictures of the introgressed genome”. With this technique, it is also possible to establish if a particular genomic region is homozygous or heterozygous, thanks to the use of molecular cytogenetic markers which are co-dominant. By this technique, it is also possible to determine in which chromosome an introgressed gene of interest is present.

According to a preferred embodiment, a plant, cell or seed of the invention comprises introgressed sequences originating from S. habrochaites in a region delimited on chromosome 8, by SNP TO-0202966 and SNP TO-0202975; this means that introgressed sequences from S. habrochaites are present in all or part of the region between the position marked by TO-0202966 and the position marked by TO-0202975 on chromosome 8. These introgressed sequences, which are the corresponding S. habrochaites sequences at the homologous position, confer the resistance to ToBRFV. According to other embodiments, the introgressed sequences conferring the resistance are to be found in the region flanked by markers TO-0202966 and TO-0202974, or by markers TO-0202968 and TO-0202975. In a particularly preferred embodiment, the introgressed sequences from S. habrochaites conferring the ToBRFV resistance are to be found within the region delimited or flanked by the markers TO-0202968 and TO-0202974.

According to still another embodiment, the QTL8 corresponds to S. habrochaites sequences, between the markers TO-0202968 and TO-0202972. In a plant, cell or seed of the invention according to this embodiment, the chromosomal segment of chromosome 8, between the markers TO-0202968 and TO-0202972, entirely corresponds to introgressed sequences from S. habrochaites. The introgressed sequences are those found in the genome of S. habrochaites, between the same markers on chromosome 8, i.e. the “corresponding” sequences.

Preferably, the introgressed sequences extend from the position corresponding to marker TO-0202968 to the position corresponding to marker TO-0202974. These introgressed sequences confer the ToBRFV resistance.

This first genetic determinant, corresponding to QTL8, is according to an embodiment chosen from the ones present in the genome of seeds of LVSTBRFVRES3. Such a genetic determinant is indeed present in the genome or genotype of these deposited seeds. A sample of these S. lycopersicum seeds has been deposited pursuant to and in satisfaction of the requirements of the Budapest Treaty, with the National collection of Industrial, Food and Marine bacteria (NCIMB) (NCIMB, Ltd, Ferguson Building, Craibstone Estate, Bucksburn, Aberdeen AB21 9YA, United Kingdom) on 20 Feb. 2023 under accession number 44125.

The QTL8 is thus, for example, obtainable from a S. lycopersicum plant grown from seeds of LVSTBRFVRES3, by crossing and selection, in order to introgress the genetic determinant conferring the ToBRFV resistance. The genome of LVSTBRFVRES3 seeds indeed comprises the QTL8 either homozygously or heterozygously, depending on the seeds, such that selection in the progeny of the plants bearing the QTL8 is necessary.

The presence of the introgressed sequences, corresponding to the QTL8 or genetic determinant on chromosome 8, conferring the resistance phenotype, can thus be identified on the basis of the phenotype, namely ToBRFV resistance, or by the use of SNP markers associated with the introgressed sequences.

Preferably, for QTL8, the presence of the introgressed sequences is thus identified or characterized in a tomato plant by one of the SNPs TO-0202968, TO-0202572, TO-0202970, TO-0202972 and TO-0202974, and preferably on the basis of at least one of TO-0202968, TO-0202572, TO-0202970 and TO-0202972. Other useful SNPs for selection of the presence of QTL8 are TO-0202966 and TO-0202975.

According to a preferred embodiment, the presence of the introgressed sequences in a tomato plant, cell or seed of the invention, obtainable from LVSTBRFVRES3 seeds or from another source comprising S. habrochaites sequences, is identifiable by at least 2, preferably at least 3, or at least 4 of said SNP markers. For example, the presence of the introgressed sequences from S. habrochaites on chromosome 8 conferring ToBRFV resistance are detected by the presence of a haplotype constituted by at least 2 SNPs. The alleles of these molecular markers, representative of the QTL or introgressed sequences conferring the resistance of the invention are reported in table A for the different SNPs on chromosome 8 disclosed. For the 5 SNPs markers mentioned above, the alleles representative of the introgressed QTL are allele G of SNP TO-0202968, allele A of TO-0202572, allele A of TO-0202970, allele A of TO-0202972 and allele A of TO-0202974.

The presence of the QTL in the genome or genotype of a tomato plant, cell or seed according to the invention can thus be detected or revealed by detecting sequences representative of the QTL, more preferably by detecting one or more of the resistant alleles of the SNPs disclosed above.

A tomato S. lycopersicum plant, cell or seed of the invention may be heterozygous or homozygous for the QTL8, or introgressed sequences of the invention conferring ToBRFV resistance. The inventors have indeed demonstrated in the experimental section that said QTL8 can confer resistance to ToBRFV in the homozygous and heterozygous state. The simultaneous detection of the susceptible and resistant allele of one or more of the markers is indicative of the presence of the QTL8 heterozygously; the sole detection of the resistant allele(s) is indicative of the presence of the QTL8 homozygously.

The inventors have moreover analyzed the introgressed sequences present in the genome of the deposited seeds LVSTBRFVRES3, and have detected the presence of different genes likely to be associated with resistance, and in particular several repeats of a gene, assumed to be responsible for the ToBRFV resistance. The inventors have thus been able to define a consensus sequence corresponding to these different repeats. This consensus sequence is set forth in SEQ ID NO:2, and codes for a resistance protein having SEQ ID NO:1.

According to other embodiments, the genetic determinant (a) mentioned above, on chromosome 8, to be combined with one of the genetic determinants (b) on chromosome 11, or with (b′) a Tm-1 gene, to confer improved ToBRFV resistance, is thus a resistance gene coding for a resistance protein having at least 90% sequence identity with SEQ ID NO.1.

The resistance gene on chromosome 8 codes for a resistance protein, which has preferably at least 90% sequence identity with SEQ ID NO.1, preferably at least 92%, at least 94%, at least 95%, at least 96%, at least 97%, at least 98%, or at least 99% sequence identity with SEQ ID NO.1. Sequence identity is measured by conventional sequence alignment programs, such as blastp or Clustal Omega, on the basis of the longer protein or by alternative conventional sequence alignment algorithms, such as the Smith-Waterman algorithm. Suitable alternative programs or algorithms are well known to the skilled person. The sequence identity is preferably defined by using the Smith-Waterman algorithm. The sequence similarity may be greater than sequence identity.

The presence of said resistance gene can be detected for example by the SNP marker TO-0202572 (SEQ ID NO.32), and especially by allele A of said marker. The flanking sequences and position of this SNP on chromosome 8 are given in Table A.

Particularly preferred resistance genes are those coding for proteins having at least 95% sequence identity to SEQ ID NO.1 or at least 96% sequence identity.

Irrespective of the sequence identity to SEQ ID NO.1, the resistance gene codes for a resistance protein, conferring resistance to ToBRFV in S. lycopersicum plants. Such a resistance is preferably conferred both at the heterozygous and homozygous state of the resistance gene; i.e. the resistance gene on chromosome 8 is preferably dominant. Recessive resistance genes are however also in the scope of the combination of the invention.

According to a preferred embodiment, the resistance gene, coming from chromosome 8 in the wild species, as described above, comprises a coding sequence having at least 80% sequence identity with SEQ NO.2, preferably at least 90%, preferably at least 95%, at the nucleotide level. The percentage of sequence identity at the nucleotide level can be determined by any appropriate algorithm or program such a blast or Smith-Waterman algorithm, or any other known suitable algorithm or program.

The resistance gene on chromosome 8 as defined above can be present either homozygously or heterozygously in a plant, seed or cell of the invention. It is preferably present heterozygously, especially as this resistance gene is expected to be dominant.

According to a preferred embodiment of the invention, the resistance gene codes for a resistance protein which restricts ToBRFV replication and/or multiplication, in case of ToBRFV infection. Preferably, such a resistance gene completely abrogates viral replication, during at least several weeks after ToBRFV infection or infestation.

Insofar as several, slightly different versions of this gene are present in the QTL8 of the invention, a plant, cell or seed advantageously comprises a resistance gene present in the genome of seeds of LVSTBRFVRES3, representative seeds of which were deposited with the NCIMB under accession number NCIMB 44125, and thus obtainable from these seeds by crossing and selection. According to another embodiment, a plant, cell or seed of the invention comprises several repeats of the resistance gene, corresponding to a consensus sequence. Advantageously, a plant, cell or seed of the invention comprises the QTL8 as defined above and present or obtainable from the seeds deposited under NCIMB 44125, in order to ensure a sufficient number and variation of the resistance gene as defined.

Regarding the other QTL or genetic determinants (b) according to the invention, conferring the improved resistance to ToBRFV when present in combination with QTL8, these QTL on chromosome 11 confer ToBRFV resistance or tolerance to the plant, when present homozygously, especially at the leaves level. In the following, any reference to ToBRFV resistance in connection with QTL11 is to be understood as referring to ToBRFV resistance or tolerance.

This resistance, or tolerance, is preferably foliar resistance, at the level of the leaves. Whereas these QTL on chromosome 11 confer resistance or tolerance to the tomato plants when present homozygously, these QTL on chromosome 11 are not necessarily present homozygously in a plant, seed or cell of the invention. Indeed, without being bound by theory, it appears that the mechanism by which the combination of one of these QTL on chromosome 11 with the resistance QTL on chromosome 8 improves the global ToBRFV resistance of the plant is not based on an additive mechanism. The QTL may thus be present heterozygously on chromosome 11 of a plant of the invention, not providing any resistance on its own in the absence of the combination with the QTL8 or resistance gene of the invention on chromosome 8.

The QTL on chromosome 11, conferring ToBRFV resistance or tolerance when present homozygously, will be referred to as QTL11 in the following.

According to a preferred embodiment, the QTL11 according to the present invention is present homozygously in the combination of the invention. According to another embodiment, it is present heterozygously.

According to an embodiment, this QTL is for example the QTL disclosed in WO2018/219941 on chromosome 11 and called “QTL3” in that document.

The QTL11 according to this embodiment is thus preferably found on chromosome 11, within a chromosomal interval or region delimited by the SNP TO-0122252 (SEQ ID NO.6) and TO-0162427 (SEQ ID NO.7). The flanking sequences and positions of these SNPs on chromosome 11 are given in Table A.

The QTL11 according to the invention to be used in combination with QTL8 or the resistance gene on chromosome 8, is chosen from the ones present in the genome or genotype of seeds of HAZTBRFVRES1, NCIMB 42758. The “QTL3” as disclosed in WO2018/219941 is indeed present in the genome or genotype of the seeds of HAZTBRFVRES1 NCIMB accession number 42758.

A sample of this S. lycopersicum seed has been deposited by Hazera Seeds Ltd. Berurim, M. P. Shikmim 79837, Israel, pursuant to and in satisfaction of the requirements of the Budapest treaty, with the National collection of Industrial, Food and Marine bacteria (NCIMB) (NCIMB, Ltd, Ferguson Building, Craibstone Estate, Bucksburn, Aberdeen AB21 9YA, United Kingdom) on 16 May 2017 under accession number 42758.

The “QTL3” as disclosed in WO2018/219941, which is an embodiment of the QTL on chromosome 11 or QTL11 according to the present invention, is also present in the genome of the seeds of LVSTBRFVRES3, NCIMB 44125.

This QTL on chromosome 11 can thus be obtained from the deposited seeds HAZTBRFVRES1 or LVSTBRFVRES3, by crossing(s) and selection(s). In order to obtain the QTL homozygously, at least a first backcross and a selfing steps are required, before selection of resistant plants. The selection can be made on the resistance phenotype, or with the SNP markers disclosed in table A, linked to this QTL.

According to another embodiment, the QTL on chromosome 11 according to the present invention, i.e. QTL11, is the QTL disclosed in WO2021/245282 on chromosome 11, and named “QTL11” in that document. QTL11 according to the present invention is thus to be distinguished from “QTL11” disclosed in WO2021/245282 which is an embodiment of the QTL11 of the present invention.

Both forms of the QTL on chromosome 11 are expected to provide the same effect when combined with the genetic determinant (a), as these different QTLs provide the same type of foliar resistance, essentially at the homozygous state.

The QTL11 according to this embodiment is thus preferably found on chromosome 11, within a chromosomal interval or region delimited by the SNP TO-0201237 (SEQ ID NO.3) and the marker SL2.50ch11_9924232 (SEQ ID NO.4), preferably by SNP TO-0201237 (SEQ ID NO.3) and SNP TO-0201241 (SEQ ID NO.5). According to this embodiment, the QTL11 according to the invention to be used in combination with QTL8 or the resistance gene on chromosome 8, is chosen from the ones present in the genome or genotype of seeds of LVSTBRFVRES2, NCIMB 43591. The “QTL11” as disclosed in WO2021/245282 is indeed present in the genome of the seeds of LVSTBRFVRES2 NCIMB accession number 43591.

A sample of these S. lycopersicum seeds has been deposited by HM.Clause S.A., rue Louis Saillant, 26800 Portes-les-Valence, France, pursuant to and in satisfaction of the requirements of the Budapest treaty, with the National collection of Industrial, Food and Marine bacteria (NCIMB) (NCIMB, Ltd, Ferguson Building, Craibstone Estate, Bucksburn, Aberdeen AB21 9YA, United Kingdom) on 1 Apr. 2020 under accession number 43591.

The QTL11 according to this embodiment corresponds to introgressed sequences from S. pimpinellifolium. The QTL is thus chosen from the introgressed sequences from S. pimpinellifolium which are to be found in chromosome 11 of the genome of the deposited seeds LVSTBRFVRES2.

This QTL on chromosome 11 can thus be obtained from the deposited seeds LVSTBRFVRES2, by crossing(s) and selection(s). In order to obtain the QTL homozygously, at least a first backcross and a selfing steps are required, before selection of resistant plants. The selection can be made on the resistance phenotype, or with the SNP markers disclosed in table A, linked to this QTL.

It is noted that some markers referred to in the description and in Table A in connection with this embodiment of QTL11, are not strictly speaking SNP, but INDEL markers, marking the insertion of a single nucleotide. For example, for marker SL2.50ch11_9924232 (SEQ ID NO.4) at position 9924233 of SL2.50 according to table A, this means that the position 9924233 is allelic, i.e. is either G or GT, corresponding to insertion of a T. The position of the “G” in SL2.50 is the position 9924233 mentioned in table A. Insofar as the allele concerns only one position for these INDEL markers, for the sake of simplicity, they can also be referred to as SNP marker in the following, by linguistic extension.

According to still a further embodiment, the QTL on chromosome 11 is the QTL on that chromosome disclosed in WO2020/018783, WO2019/110130 or WO2019/110821.

The QTL on chromosome 11 disclosed in WO2018/219941 and WO2021/245282 have been identified and mapped mainly by identifying the presence of sequences representative of the introgressed QTL or resistance/tolerance QTL at different loci along the region of chromosome 11 mentioned above, namely:
at 12 different loci defined by the 12 SNPs TO-0122252 (SEQ ID NO.6), TO-0144317 (SEQ ID NO.8), TO-0142270 (SEQ ID NO.9), TO-0142294 (SEQ ID NO.10), TO-0142303 (SEQ ID NO.11), TO-0142306 (SEQ ID NO.12), TO-0181040 (SEQ ID NO.14), TO-0123057 (SEQ ID NO.15), TO-0125528 (SEQ ID NO.16), TO-0162432 (SEQ ID NO.17) and TO-0162427 (SEQ ID NO.7) for “QTL3” of WO2018/219941 and
at 9 different loci defined by the 9 markers SNPs TO-0201237 (SEQ ID NO.3), TO-0201238 (SEQ ID NO.18), TO-0201239 (SEQ ID NO.19), TO-0201240 (SEQ ID NO.20), TO-0201241 (SEQ ID NO.5), SL2.50ch11_9684449 (SEQ ID NO.21), SL2.50ch11_9779896 (SEQ ID NO.22), SL2.50ch11_9823405 (SEQ ID NO.23) and SL2.50ch11_9924232 (SEQ ID NO.4) for “QTL11” of WO2021/245282.

Preferred SNP amongst these SNPs is the SNP or marker having SEQ ID NO. 16 (TO-0125528).

The presence of the introgressed sequences, or resistance/tolerance QTL, conferring the resistance/tolerance phenotype can thus be identified on the basis of these SNP markers, in the genome of a plant, seed or cell of the invention. They can also be used for selecting plants comprising the QTL on chromosome 11 in their genotype.

Preferably, the presence of a QTL on chromosome 11 according to WO2018/219941 or WO2021/245182 can be characterized by the detection of at least one of the resistant alleles of the markers having SEQ ID NO.3 to 23, as disclosed in the last column of Table A. According to a preferred embodiment, the presence of a QTL on chromosome 11, or QTL11 according to this invention, is characterized:
by the detection of at least one, preferably at least 2, more preferably at least 4, at least 5 or at least 6 of allele CT of SL2.50ch11_9684449, allele AT of SL2.50ch11_9779896, allele C of SL2.50ch11_9823405, allele GT of SL2.50ch11_9924232, allele G of TO-0201237, allele A of TO-0201238, allele A of TO-0201239, allele A of TO-0201240 and allele A of TO-0201241, for “QTL11” or
by the detection of at least one, preferably at least 2, more preferably at least 4, at least 5 or at least 6 of allele T of TO-0122252, allele C of TO-0144317, allele T of TO-0142270, allele G of TO-0142294, allele A of TO-0142303, allele A of TO-0142306, allele G of TO-0181040, allele G of TO-0123057, allele A of TO-0125528, allele C of TO-0162432 and allele T of TO-0162427 for “QTL3”.

A plant, seed or cell of the invention is preferably characterized by the presence in its genome of the “QTL3” as defined in WO2018/219941, namely by the presence of at least one of the following alleles, which under specific conditions are representative of the presence of said QTL, namely at least one of: allele T of TO-0122252 and/or allele C of TO-0144317 and/or allele T of TO-0142270 and/or allele G of TO-0142294 and/or allele A of TO-0142303 and/or allele A of TO-0142306 and/or allele G of TO-0181040 and/or allele G of TO-0123057 and/or allele A of TO-0125528 and/or allele C of TO-0162432 and/or allele T of TO-0162427.

In order to comprise in its genome the “QTL3” as defined, a plant, seed or cell of the invention is, according to an embodiment, a progeny of an hybrid between a plant grown from the seeds of HAZTBRFVRES1 (NCIMB accession number 42758) or from LVSTBRFVRES3 (NCIMB accession number 44125) and a S. lycopersicum plant bearing the resistance QTL or gene on chromosome 8 as defined above.

According to another embodiment, a plant, seed or cell of the invention is preferably characterized by the presence in its genome of the “QTL11” as defined in WO2021/245282, wherein the presence of said “QTL11” on chromosome 11 is identified by at least one of the following alleles: allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and allele GT of SL2.50ch11_9924232, preferably by at least one of allele G of TO-0201237, allele A of TO-0201238, allele A of TO-0201239, allele A of TO-0201240 and allele A of TO-0201241.

In order to comprise in its genome the “QTL11” as defined, a plant, seed or cell of the invention is, according to another embodiment, a progeny of an hybrid between a plant grown from the seeds of LVSTBRFVRES2 (NCIMB accession number 43591) and a S. lycopersicum plant bearing the resistance QTL or gene on chromosome 8 as defined above.

These markers and/or resistant alleles are applicable to all the different aspects of the present invention.

The inventors have also shown that the resistance to ToBRFV can be still improved by addition of the Tm-1 resistance gene, as mentioned in WO2020/249798.

The Tm-1 gene is as defined inter alia in the publication Ishibashi et al, 2007; preferably ‘Tm-1 gene’ refers to a genetic sequence encoding a protein having the Tm-1 activity reported in the article, namely the ability to inhibit the viral replication of a wild-type ToMV strain Tm-1 sensitive, for example the strain ToMV-L disclosed in this article. According to a preferred embodiment, the Tm-1 gene according to the invention is a gene encoding a protein having the 754 amino acid sequence reported in Ishibashi et al, corresponding to SEQ ID NO.24 (NCBI BAF75724), or a protein having at least 75%, preferably at least 80%, more preferably at least 85%, 90%, or 95% sequence identity with SEQ ID NO:24 and exhibiting the Tm-1 activity reported in Ishibashi et al, 2007, namely the ability to inhibit viral RNA replication of a wild-type Tm-1 sensitive ToMV strain. According to a preferred embodiment, this gene has a sequence corresponding to the mRNA sequence referred to in Ishibashi et al, 2007, namely sequence NCIB AB287296 (SEQ ID NO.25), or a sequence having at least 50%, preferably at least 60%, at least 70%, more preferably at least 75%, 80%, 85%, 90%, or 95% sequence identity with SEQ ID NO.25. Irrespective of the degree of sequence identity with SEQ ID NO.25, a Tm-1 gene according to the invention preferably encodes a protein exhibiting the Tm-1 activity reported in Ishibashi et al, 2007, namely the ability to inhibit viral RNA replication of wild-type ToMV.

It is preferred that, in the genome or genotype of a plant, seed or cell of the invention, the Tm-1 gene be present on chromosome 2. The present invention however also encompasses plant, seed or cell, comprising the Tm-1 gene at a locus which does not correspond to the locus mentioned in Ishibashi et al, 2007.

The Tm-1 can be present homozygously or heterozygously in a plant, seed or cell of the invention, according to this embodiment.

A suitable marker which can be used for determining the presence of the Tm-1 gene, especially in progeny segregating for this feature is the SNP TO-0178073 (SEQ ID NO.31). The sequence of this marker is indicated in table A. Allele A of this SNP is indeed indicative of the presence of the resistance Tm-1 gene.

As disclosed above, the Tm-1 gene as defined can be combined with the genetic determinant (a), as genetic determinant (b′) in place or in addition to genetic determinant (b) as defined above. Tm-1 gene can be present homozygously or heterozygously.

The inventors have also shown that the resistance to ToBRFV can be still improved by addition of a resistance QTL on chromosome 9, providing resistance to ToBRFV when present homozygously, such as the “QTL2” disclosed in WO2018/219941 or the “QTL9” disclosed in WO2021/245282.

The invention is thus also directed to tomato plant, seed or cell, comprising homozygously or heterozygously the combination of genetic determinants as disclosed above, as well as at least one or more of the following additional genetic elements:
the “QTL2” on chromosome 9, as disclosed in WO2018/219941; heterozygously or homozygously; or
the “QTL9” on chromosome 9, as disclosed in WO2021/245282, preferably heterozygously or homozygously.

These additional QTLs are defined in the mentioned patent applications; their characterizing features are not repeated here but are incorporated by reference.

Appropriate sources for introgressing said QTLs in tomato plants are plants grown from seeds HAZTBRFVRES1 (NCIMB 42758) for the QTL2 on chromosome 9, as disclosed in WO2018/219941, and plants grown from seeds LVSTBRFVRES2 (NCIMB 43591) for the QTL9 on chromosome 9 as disclosed in WO2021/245282. Said QTLs can thus be obtained from these deposited seeds, by crossing and selection.

Suitable markers for detecting the presence of said QTLs, and/or for selecting plants comprising these QTLs, are inter alia:
TO-0180955 (SEQ ID NO:42), TO-0196724 (SEQ ID NO:43), TO-0145125 (SEQ ID NO:44) and TO-0196109 (SEQ ID NO:45) for QTL2 as disclosed in WO2018/219941, and
TO-0201220 (SEQ ID NO:46), TO-0201221 (SEQ ID NO:47), TO-0201222 (SEQ ID NO:48), TO-0201223 (SEQ ID NO:49), TO-0201224 (SEQ ID NO:50), TO-0201225 (SEQ ID NO:51), TO-0201226 (SEQ ID NO:52), TO-0201227 (SEQ ID NO:53), TO-0201228 (SEQ ID NO:54), TO-0201229 (SEQ ID NO:55), TO-0201230 (SEQ ID NO:56), TO-0201231 (SEQ ID NO:57), TO-0201232 (SEQ ID NO: 58) and TO-0201233 (SEQ ID NO:59), for QTL9 as disclosed in WO2021/245282.

The alleles of these SNPs more specifically linked to the QTLs providing resistance are:
allele G of TO-0180955, allele C of TO-0196724, allele G of TO-0145125 and allele G of TO-0196109 for the QTL2, and
allele G of SNP TO-0201220, allele G of TO-0201221, allele A of TO-0201222, allele A of TO-0201223, allele A of TO-0201224, allele A of TO-0201225, allele A of TO-0201226, allele C of TO-0201227, allele C of TO-0201228, allele A of TO-0201229, allele C of TO-0201230, allele C of TO-0201231, allele G of TO-0201232 and allele G of TO-0201233 for the QTL9.

According to still another embodiment of the invention, the combination of genetic determinants as defined, namely resistance QTL or resistance gene on chromosome 8, with QTL11, potentially in combination with Tm-1 and/or QTL9, or QTL or resistance gene on chromosome 8, with Tm-1 gene, allows the plant of the invention to delay, reduce or inhibit the replication or multiplication of the ToBRFV, in the absence of infection symptoms, on the leaves and fruits, during at least 5 months after the infection, irrespective of the infection time. The reduction or inhibition in viral replication can be assayed by any appropriate means, especially by ELISA, quantitative PCR and bioassay on tobacco as illustrated in the experimental section. The level of viral reduction, or the period during which this reduction is observed is in any event superior to the level or period applicable to a plant comprising only one of the two genetic determinants of the invention, without the other one.

In another embodiment, the combination of genetic determinants as defined, allows the plant of the invention to delay, reduce or inhibit the replication or multiplication of the ToBRFV, in the absence of infection symptoms, even at temperature above 25° C., preferably even at temperature of 28° C. or above, or 30° C., especially at daytime and nighttime temperatures above 25° C., especially above 28° C. Resistance QTL on chromosome 8, in the absence of a QTL11, cannot delay, reduce or inhibit the replication or multiplication of the ToBRFV, when temperature is above 28° C., especially above 30° C. or above 33° C.; especially when the nighttime temperature is of 28° C. or more.

In still another embodiment, the combination of genetic determinants as defined, allows the plant of the invention to have almost no non-marketable fruits in case of ToBRFV infestation of the culture, and in any event a percentage of non-marketable fruits due to ToBRFV infestation which is below the percentage of non-marketable fruits of a plant not bearing the combination of genetic determinants according to the invention. Especially, resistant plants as disclosed herein bear tomatoes such that at least 70% of the fruits are marketable at maturity, i.e. have no discoloration spots, no browning calyx, are not undersized with a rough surface and have no brown, necrotic spots. Preferably, at least 80% of the fruits remain marketable at maturity, preferably at least 90% of the fruits, even in case of a double infection, and also even in case of infection at any early stage of plant development, contrary to plants comprising only the resistance QTL on chromosome 8, or only one of the QTL on chromosome 11, which have a lower percentage of marketable fruits at maturity, due to ToBRFV infestation.

A plant, seed or cell of the invention thus comprises in its genome the combination of genetic determinants as already defined, namely a resistance gene or resistance QTL on chromosome 8 in combination with a QTL on chromosome 11, and advantageously also the Tm-1 resistance gene and/or a QTL9, said combination providing the increased or enhanced ToBRFV resistance. A plant, seed or cell according to the invention preferably comprises one of the following combinations of genetic determinants:
a) The resistance gene or QTL on chromosome 8 heterozygously, and QTL on chromosome 11 homozygously;
b) The resistance gene or QTL on chromosome 8 homozygously, and QTL on chromosome 11 homozygously;
c) The resistance gene or resistance QTL on chromosome 8 heterozygously, and QTL on chromosome 11 heterozygously;
d) The resistance gene or QTL on chromosome 8 homozygously, and QTL on chromosome 11 heterozygously;
e) The resistance gene or QTL on chromosome 8 heterozygously, QTL on chromosome 11 homozygously and Tm-1 and/or a QTL9 heterozygously;
f) The resistance gene or QTL on chromosome 8 homozygously, QTL on chromosome 11 homozygously and Tm-1 and/or a QTL9 heterozygously;
g) The resistance gene or QTL on chromosome 8 heterozygously, QTL on chromosome 11 heterozygously and Tm-1 and/or a QTL9 heterozygously;
h) The resistance gene or QTL on chromosome 8 homozygously, QTL on chromosome 11 heterozygously and Tm-1 and/or a QTL9 heterozygously;
i) The resistance gene or QTL on chromosome 8 heterozygously, QTL on chromosome 11 homozygously and Tm-1 and/or a QTL9 homozygously;
j) The resistance gene or QTL on chromosome 8 homozygously, QTL on chromosome 11 homozygously and Tm-1 and/or a QTL9 homozygously;
k) The resistance gene or QTL on chromosome 8 heterozygously, QTL on chromosome 11 heterozygously and Tm-1 and/or a QTL9 homozygously; and
l) The resistance gene or QTL on chromosome 8 homozygously, QTL on chromosome 11 heterozygously and Tm-1 h and/or a QTL9 homozygously.

In the preceding combinations, and in the different embodiments of the invention, the resistance QTL on chromosome 8 may be replaced by a resistance gene as defined.

As already mentioned, “the resistance gene on chromosome 8” is to be understood a gene coding for a resistance protein having at least 90% sequence identity with SEQ ID NO.1.

Moreover, as stressed previously, the invention also concerns plant, seed or cell comprising one of the following combinations of genetic determinants:
i) The resistance gene or resistance QTL on chromosome 8 heterozygously, and Tm-1 gene homozygously;
ii) The resistance gene or resistance QTL on chromosome 8 homozygously, and Tm-1 gene homozygously;
iii) The resistance gene or resistance QTL on chromosome 8 heterozygously, and Tm-1 gene heterozygously;
iv) The resistance gene or resistance QTL on chromosome 8 homozygously, and Tm-1 gene heterozygously.

A S. lycopersicum plant, seed or cell according to the invention preferably also comprises a further gene or QTL providing one or more of additional resistances, for example the Tm-2 gene, especially Tm-22 allele. In still a further embodiment, a plant, seed or cell of the invention further comprises the Tm-2 resistance gene, on chromosome 9, preferably heterozygously; especially the Tm-22 allele of Tm-2 or any alternative allele conferring resistance to TMV and ToMV.

Indeed, a S. lycopersicum plant according to the invention is preferably a commercial plant or line, or a cell or seed thereof. Such a commercial plant or line thus preferably also exhibits resistance to ToMV (tomato mosaic virus), for example due to the presence of a Tm-2 gene (allele Tm-2 or Tm-22 (also known as Tm-2a)) which also confers resistance to TMV (Tobacco Mosaic Virus). A plant according to this aspect of the invention preferably comprises the Tm-22 gene or an analog thereof providing ToMV and TMV resistances. The Tm-22 gene is well known to those skilled in the art; a suitable sequence for this gene is referred to as Solyc09g018220, or GeneBank AF536201, and AAQ10736 for the protein sequence. Variants and analogs are well known in the field of the invention and do not need to be described here.

A plant, cell or seed according to this aspect may also have genetic determinants providing the following additional features: nematode resistance trait (Mi-1 or Mi-j), as well as Fusarium and Verticillium resistances, and TYLCV resistance.

Other resistances or tolerances are also envisaged according to the invention.

Moreover, the commercial plant of the invention gives rise to fruits in suitable conditions, which are at least 10 grams, preferably 25 grams at full maturity, preferably at least 100 g at full maturity and or even more preferably at least 150 g or at least 200 g at full maturity. The number of fruits per plant is moreover essentially unaffected by the presence of the combination of genetic determinants of the invention, i.e. the productivity of a plant according to the invention is not inferior by more than 20% to a plant having the same genotype but devoid of said combination. A plant of the invention thus generally bears at least 3, preferably around 4 tomatoes per cluster, and these fruits have preferably a weight between 150 g and 180 g.

According to still another embodiment, a plant of the invention is a determinate, indeterminate or semi-indeterminate plant, or seed or cell thereof, i.e. corresponding to determinate, indeterminate or semi-indeterminate growth habit.

By determinate, it is meant tomato plants which tend to grow their foliage first, then set flowers that mature into fruit if pollination is successful. All of the fruits tend to ripen on a plant at about the same time. Indeterminate tomatoes start out by growing some foliage, then continue to produce foliage and flowers throughout the growing season. These plants will tend to have tomato fruit in different stages of maturity at any given time. The semi-determinate tomatoes have a phenotype between determinate and indeterminate, they are typical determinate types except that grow larger than determinate varieties.

According to still another embodiment, a plant of the invention is used as a scion or as a rootstock in a grafting process. Grafting is a process that has been used for many years in crops such as cucurbitacea, but only more recently for tomato. Grafting may be used to provide a certain level of resistance to telluric pathogens such as Phytophthora or to certain nematodes. Grating is therefore intended to prevent contact between the plant or variety to be cultivated and the infested soil. The variety of interest used as the graft or scion, optionally an F1 hybrid, is grafted onto the resistant plant used as the rootstock. The resistant rootstock remains healthy and provides, from the soils, the normal supply for the graft that it isolates from the diseases.

As detailed above, the invention is directed to S. lycopersicum plants, exhibiting the improved ToBRFV resistance due to the combination of genetic determinants, as well as to seeds giving rise to those plants, and cells of these plants or seeds, or other plant parts, comprising said combination in their genome or genotype, and to progeny of such a plant of the invention, the progeny comprising said combination of genetic determinants in its genome or genotype.

Progeny encompasses the first generation, the second, and all further descendants from a cross with a plant according to the invention, wherein a cross comprises a cross with itself, i.e. a selfing; or a cross with another plant. A plant or seed according to the invention may be a progeny or offspring of a plant grown from the deposited seeds LVSTBRFVRES2, deposited at the NCIMB under the accession number NCIMB 43591, and/or from the deposited seeds HAZTBRFVRES1, deposited at the NCIMB under the accession number NCIMB 42758. Plants grown from these deposited seeds indeed comprise one of the QTL on chromosome 11 of the invention conferring the improved phenotype when combined with the resistance QTL on chromosome 8. They can be used to transfer one of the QTL on chromosome11 into another background comprising the resistance QTL on chromosome 8 by crossing and selfing and/or backcrossing. The plant of the invention may thus be a progeny of an hybrid between a plant grown from the seeds of HAZTBRFVRES1 (NCIMB accession number 42758) and a S. lycopersicum plant bearing the resistance QTL8 on chromosome 8 as defined above, such as a plant grown from the seeds of LVSTBRFVRES3 (NCIMB 44125), or may a progeny of an hybrid between a plant grown from the seeds of LVSTBRFVRES2 (NCIMB accession number 43591) and a S. lycopersicum plant bearing the resistance QTL on chromosome 8 as defined above, such as a plant grown from the seeds of LVSTBRFVRES3 (NCIMB 44125), or may be one of these hybrids.

Alternatively, the plant of the invention may be a plant or a progeny or offspring of a plant grown from the deposited seeds LVSTBRFVRES3, deposited at the NCIMB under the accession number NCIMB 44125. Regarding the deposited seeds NCIMB 44125, NCIMB 42758 and NICMB 43591, it is noted that these seeds do not correspond to plant varieties, they are not homozygous for most of the genes except for the QTL on chromosome 11; their phenotype is thus not fixed during propagation, except for the ToBRFV resistance/tolerance QTL on chromosome 11; most of their phenotypic traits segregate during propagation, with the exception of ToBRFV resistance, especially the QTL on chromosome 11.

The progeny of offspring mentioned above relates to progeny selected for the presence of the combination of genetic determinants according to the invention.

It is noted that the seeds or plants of the invention may be obtained by different processes and are not exclusively obtained by means of an essentially biological process.

According to such an aspect, the invention relates to a tomato plant or seed, preferably a non-naturally occurring tomato plant or seed, which may comprise one or more mutations in its genome, which provides the plant with an improved resistance to ToBRFV, which mutations correspond to a resistance QTL on chromosome 8 and one of the QTL on chromosome 11 as defined according to the invention.

In another embodiment, the invention relates to a method for obtaining a tomato plant or seed carrying one or more mutations in its genome, which provides the plant with an improved resistance to ToBRFV as defined according to the invention. Such a method is illustrated in example 11 and may comprise:
a) treating M0 seeds of a tomato plant to be modified with a mutagenic agent to obtain M1 seeds;
b) growing plants from the thus obtained M1 seeds to obtain M1 plants;
c) producing M2 seeds by self-fertilisation of M1 plants; and
d) optionally repeating step b) and c) n times to obtain M1+n seeds.

The M1+n seeds are grown into plants and submitted to ToBRFV infection. The surviving plants, or those with the milder symptoms of ToBRFV infection, are multiplied one or more further generations while continuing to be selected for their fruit and/or foliar resistance to ToBRFV.

In this method, the M1 seeds of step a) can be obtained via chemical mutagenesis such as EMS mutagenesis. Other chemical mutagenic agents include but are not limited to, diethyl sufate (des), ethyleneimine (ei), propane sultone, N-methyl-N-nitrosourethane (mnu), N-nitroso-N-methylurea (NMU), N-ethyl-N-nitrosourea (enu), and sodium azide.

Alternatively, the mutations are induced by means of irradiation, which is for example selected from x-rays, fast neutrons, UV radiation.

In another embodiment of the invention, the mutations are induced by means of genetic engineering. Such mutations also include the integration of sequences conferring the ToBRFV fruit and/or foliar resistance, as well as the substitution of residing sequences by alternative sequences conferring the ToBRFV fruit and/or foliar resistance. Preferably, the mutations are the integration of a resistance QTL on chromosome 8, and one of the QTL on chromosome 11, as described above, in replacement of the homologous sequences of a S. lycopersicum plants. According to an embodiment, the mutation is the substitution of the sequence comprised within SNP TO-0201237 (SEQ ID NO.3) and SL2.50ch11_9924232 (SEQ ID NO.4) on chromosome 11 of S. lycopersicum genome, or a fragment thereof such as the sequence comprised within SNP TO-0201237 and SNP TO-0201241, by the homologous sequence on chromosome 11 present in the genome of a plant of which a representative sample was deposited with the NCIMB under deposit number NCIMB 43591, wherein the sequence or fragment thereof confers resistance to ToBRFV when present homozygously. According to another embodiment, the mutation is the substitution of the sequence comprised within SNP TO-0122252 and TO-0162427 on chromosome 11 of S. lycopersicum genome, or a fragment thereof, by the homologous sequence on chromosome 11 present in the genotype of a plant of which a representative sample was deposited with the NCIMB under deposit number NCIMB 42758, wherein the sequence or fragment thereof confers resistance to ToBRFV when present homozygously. In an embodiment, the mutation is the substitution of the sequence comprised within SNP TO-0202968 and TO-0202972 on chromosome 8 of S. lycopersicum genome, or a fragment thereof, by the homologous sequence on chromosome 8 present in the genotype of a plant of which a representative sample was deposited with the NCIMB under deposit number NCIMB 44125, wherein the sequence or fragment thereof confers resistance to ToBRFV.

The genetic engineering means which can be used include the use of all such techniques called New Breeding Techniques which are various new technologies developed and/or used to create new characteristics in plants through genetic variation, the aim being targeted mutagenesis, targeted introduction of new genes or gene silencing (RdDM). Example of such new breeding techniques are targeted sequence changes facilitated through the use of Zinc finger nuclease (ZFN) technology (ZFN-1, ZFN-2 and ZFN-3, see U.S. Pat. No. 9,145,565), Oligonucleotide directed mutagenesis (ODM), Cisgenesis and intragenesis, Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641), engineered meganuclease, re-engineered homing endonucleases, DNA guided genome editing (Gao et al., 2016), and Synthetic genomics. A major part of targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). A complete description of each of these techniques can be found in the report made by the Joint Research Center (JRC) Institute for Prospective Technological Studies of the European Commission in 2011 and titled “New plant breeding techniques-State-of-the-art and prospects for commercial development”.

The invention in another aspect also concerns any plant likely to be obtained from seed or plants of the invention as described above, and also plant parts of such a plant, and most preferably explant, scion, cutting, seed, fruit, root, rootstock, pollen, ovule, embryo, protoplast, leaf, anther, stem, petiole, cotyledon, flower, root tip, hypocotyl and any other plants part, wherein said plant, explant, scion, cutting, seed, fruit, root, rootstock, pollen, ovule, embryo, protoplast, leaf, anther, stem, petiole, cotyledon, flower, root tip, hypocotyl and/or plant part is obtainable from a seed or plant according to the first aspect of the invention, i.e. bearing the combination of genetic determinants of the invention in their genome. This plant part, inter alia explant, scion, cutting, seed, fruit, root, rootstock, pollen, ovule, embryo, protoplast, leaf, anther, stem, petiole, cotyledon, flower, root tip or hypocotyl, comprises in its genome the resistance QTL on chromosome 8 as defined above, or the corresponding resistance gene, as well as one of the QTL on chromosome 11, the combination conferring the phenotype of interest, i.e. improved resistance to ToBRFV.

According to a preferred embodiment, the invention is directed to seed as described above, which develops into a plant according to the first aspect of the invention, thus having an improved resistance against ToBRFV infection thanks to the presence of the combination of genetic determinants as defined above.

The QTL on chromosome 11, also referred to as QTL11 In the present description, and resistance QTL or gene on chromosome 8 referred to in this aspect of the invention are the ones defined above in the context of plants of the invention. The resistance gene on chromosome 8 is a gene coding for a resistance protein having at least 90% sequence identity with SEQ ID NO.1.

The different features of the QTLs defined in relation with the first aspect of the invention apply mutatis mutandis to this aspect of the invention. The QTL8 is thus preferably chosen from those present in the genome of a plant corresponding to the deposited material LVSTBRFVRES3 (NCIMB accession number 44125), and thus obtainable from these seeds, by crossing and selection. It is advantageously characterized by the presence of at least one of the resistance alleles of the SNPs of table A on chromosome 8, preferably by the presence of allele G of SNP marker TO-0202968, allele A of TO-0202572, allele A of SNP marker TO-0202970, allele A of SNP marker TO-0202972 or allele A of SNP marker TO-0202974.

The QTL on chromosome 11 is also preferably chosen from those present in the genome of a plant corresponding to the deposited material LVSTBRFVRES3 (NCIMB accession number 44125), LVSTBRFVRES2 (NCIMB accession number 43591) or HAZTBRFVRES1 (NCIMB accession number 42758), and thus obtainable from these seeds, by crossing and selection. It is advantageously characterized by the presence of at least one of the resistance alleles of the SNPs of table A on chromosome 11; preferably by the presence of allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 or allele GT of SL2.50ch11_9924232; e.g. by the presence of at least one of allele G of TO-0201237, allele A of TO-0201238, allele A of TO-0201239, allele A of TO-0201240 and allele A of TO-0201241. Alternatively, it may be characterized by the presence of at least one of allele T of TO-0122252, allele C of TO-0144317, allele T of TO-0142270, allele G of TO-0142294, allele A of TO-0142303, allele A of TO-0142306, allele G of TO-0181040, allele G of TO-0123057, allele A of TO-0125528, allele C of TO-0162432 and allele T of TO-0162427.

The invention is also directed to a cell of a S. lycopersicum plant, such that this cell comprises, in its genome, the combination of genetic determinants of the present invention conferring the improved resistance to ToBRFV to a S. lycopersicum plant. The combination of genetic determinants is the one already defined in the frame of the present invention, it is characterized by the same features and preferred embodiments already disclosed with respect to the plants and seeds according to the preceding aspects of the invention. The presence of the genetic determinants, responsible for the improved phenotype, can be revealed by the techniques disclosed above and well known to the skilled reader.

Cells according to the invention can be any type of S. lycopersicum cell, inter alia an isolated cell and/or a cell capable of regenerating a whole S. lycopersicum plant, bearing the combination of genetic determinants of the invention.

The present invention is also directed to a tissue culture of non-regenerable or regenerable cells of the plant as defined above according to the present invention; preferably, the regenerable cells are derived from embryos, protoplasts, meristematic cells, callus, pollen, leaves, anthers, stems, petioles, roots, root tips, fruits, seeds, flowers, cotyledons, and/or hypocotyls of the invention, and the cells contain in their genome the combination of genetic determinants of the invention, which confers the improved resistance to ToBRFV, at least with respect to each genetic determinant separately.

Preferably, such a cell also comprises the Tm-1 gene, as defined in the context of the present invention, either homozygously or heterozygously, ans/or the QTL9. The resistance QTL on chromosome 8 and the QTL on chromosome 11 can be present, independently, either homozygously or heterozygously. Different combinations of these genetic determinants are disclosed above and applicable to the present aspect of the invention.

Such a cell also advantageously comprises any additional resistance or tolerance gene, as disclosed in the context of the first aspect of the invention, also applicable here.

The tissue culture will preferably be capable of regenerating plants having the physiological and morphological characteristics of the foregoing tomato plant, and of regenerating plants having substantially the same genotype as the foregoing tomato plant. The present invention also provides tomato plants regenerated from the tissue cultures of the invention.

The invention also provides a protoplast of the plant defined above, or from the tissue culture defined above, said protoplast containing the combination of genetic determinants of the invention, conferring the improved phenotype as defined.

The invention is also directed to tissue of a plant of the invention; the tissue can be an undifferentiated tissue, or a differentiated tissue. Such a tissue comprises one or more cells comprising the combination of genetic elements of the invention.

The invention is also directed to propagation material, capable of producing a resistant tomato plant according to the invention, comprising the combination of genetic determinants or elements as defined above. Particularly preferred propagation material is seed. According to an embodiment, the invention is thus directed to seed of S. lycopersicum plant, which develops into a plant according to the invention, or which derives from a plant according to the invention and which comprises in its genome the combination of genetic determinants as disclosed.

The seed according to the invention are preferably coated or pelleted with individual or combined active species such as plant nutrients, enhancing microorganisms, or products for disinfecting the environment of the seeds and plants. Such species and chemicals may be a product that promotes the growth of plants, for example hormones, or that increases their resistance to environmental stresses, for example defense stimulators, or that stabilizes the pH of the substrate and its immediate surroundings, or alternatively a nutrient.

They may also be a product for protecting against agents that are unfavorable toward the growth of young plants, including herein viruses and pathogenic microorganisms, for example a fungicidal, bactericidal, hematicidal, insecticidal or herbicidal product, which acts by contact, ingestion or gaseous diffusion; it is, for example, any suitable essential oil, for example extract of thyme. All these products reinforce the resistance reactions of the plant, and/or disinfect or regulate the environment of said plant. They may also be a live biological material, for example a nonpathogenic microorganism, for example at least one fungus, or a bacterium, or a virus, if necessary with a medium ensuring its viability; and this microorganism, for example of the Pseudomonas, Bacillus, Trichoderma, Clonostachys, Fusarium, Rhizoctonia, etc. type stimulates the growth of the plant, or protects it against pathogens.

According to other aspects, the present invention is also directed to the use of plants or seed of LVSTBRFVRES3, deposited at the NCIMB under the accession number NCIMB 44125, and to plants derived therefrom, comprising the combination of genetic determinants according to the invention, as a breeding partner in a breeding program aiming at introgressing these genetic determinants in S. lycopersicum plants, for obtaining plants having the improved phenotype of the invention. The invention is also directed to the use of plants or seed of LVSTBRFVRES2, deposited at the NCIMB under the accession number NCIMB 43591, and to plants derived therefrom, comprising homozygously one of the QTL on chromosome 11, as a breeding partner in a breeding program aiming at cumulating or pyramiding this QTL with a resistance QTL or gene on chromosome 8, for obtaining S. lycopersicum plants having the improved phenotype of the invention. Indeed, such a breeding partner harbors homozygously in its genome a QTL on chromosome 11, or QTL11, conferring ToBRFV resistance when present homozygously. By crossing this plant with a tomato plant, especially a line comprising the resistance QTL or gene on chromosome 8, as defined according to the invention, it is thus possible to combine a QTL on chromosome 11 and the resistance QTL or gene conferring the desired phenotype to the progeny.

The invention is also directed to the same use with plants or seed of HAZTBRFVRES1, deposited at the NCIMB under the accession number NCIMB 42758, and to plants derived therefrom, comprising the QTL11 homozygously.

The invention is also directed to use of not only the deposited seeds and plants, but also plants or seeds of the invention, comprising the combination of genetic determinants, as a breeding partner or introgression partner in a breeding program for obtaining other S. lycopersicum plants having the improved phenotype of the invention. The combination of genetic determinants will advantageously be introduced into varieties that contain other desirable genetic traits such as resistance to disease, early fruit maturation, drought tolerance, fruit shape, and the like. Preferably, the combination will advantageously be introduced into plants or varieties comprising the Tm-1 gene, and potentially also the Tm-22 gene.

In such a breeding program, the selection of the progeny displaying the desired phenotype, or bearing the combination of genetic determinants linked to the desired phenotype, can advantageously be carried out on the basis of the alleles of the SNP markers mentioned above, for QTL11 and for the resistance QTL or gene on chromosome 8.

The selection can indeed be made on the basis of the presence of any one of the resistant alleles of the SNPs linked to the genetic determinants providing improved phenotype or a combination of these alleles.

Such selection will be made on the presence of the alleles of interest in a genetic material sample of the plant to be selected. The presence of this or these allele(s) indeed may confirm the presence of the QTL8 and/or one of the QTL on chromosome 11, at the loci defined by said SNPs. Following point mutation or recombination event, it is however conceivable that at least 1 or 2 of these alleles is lost, the remaining of the chromosomal fragment bearing the QTL8 or QTL11 of interest still conferring the phenotype of interest, when combined.

A plant according to the invention, or grown from a seed of the invention, is thus particularly valuable in a marker assisted selection for obtaining commercial tomato lines and varieties, having the improved phenotype of the invention.

According to still another aspect, the invention also concerns methods or processes for the production or breeding of S. lycopersicum plants, having the desired phenotype, especially commercial plants and inbred parental lines. The present invention is indeed also directed to transferring the combination of genetic determinants of the invention conferring the improved ToBRFV resistance to other tomato plants, especially other tomato varieties, or other species or inbred parental lines, and is useful for producing new types and varieties of tomato.

In this regard, the invention also comprises methods for breeding S. lycopersicum plants having improved ToBRFV resistance, comprising the steps of crossing a plant bearing a QTL on chromosome 11, conferring ToBRFV resistance, especially when present homozygously, for example a plant grown or obtainable from the deposited seeds LVSTBRFVRES2 or HAZTBRFVRES1, or progeny thereof bearing the QTL11 conferring ToBRFV resistance, with another S. lycopersicum plant comprising a resistance QTL on chromosome 8, as defined above, providing also ToBRFV resistance, for example a plant grown or obtainable from the deposited seeds LVSTBRFVRES3, or progeny thereof bearing the QTL8 conferring ToBRFV resistance. The QTLs are as defined above. The QTL8 may be a resistance gene as defined according to the invention.

According to some embodiments, the invention thus concerns a method or process for the production of a plant having increased ToBRFV resistance comprising the following steps:
a) Crossing a plant grown from a deposited seed NCIMB 43591 or NCIMB 42758, or progeny thereof, comprising a QTL on chromosome 11 conferring ToBRFV resistance or tolerance when present homozygously, or a plant bearing the Tm-1 gene, with a S. lycopersicum plant, comprising a resistance gene coding for a resistance protein having at least 90% sequence identity with SEQ ID NO.1 according to the invention,
b) Selecting a plant in the progeny thus obtained, bearing the QTL on chromosome 11 and/or the Tm-1 gene, and the resistance gene;
c) Optionally self-pollinating one or several times the plant obtained at step b) and selecting in the progeny thus obtained a plant having an improved resistance to ToBRFV.

Alternatively, the method or process may comprise instead of step a) the following steps:
a1) Crossing a plant corresponding to the deposited seeds (NCIMB 43591 or NCIMB 42758), or progeny thereof, comprising a QTL on chromosome 11, or QTL11, conferring ToBRFV resistance or tolerance when present homozygously, or a plant bearing the Tm-1 gene, with a S. lycopersicum plant comprising said resistance gene,
a2) Increasing the F1 hybrid by means of selfing to create F2 population.

In the above methods or processes, SNPs markers are preferably used in steps b) and/or c), for selecting plants bearing sequences conferring the resistance phenotype of interest.

The SNP markers are preferably as disclosed in connection with the preceding aspects of the invention.

By selecting a plant on the basis of the allele of one or more SNPs, it is to be understood that the plant is selected when the allele of the SNP(s) is (are) the allele corresponding to the “resistant” or “tolerant” allele of the SNP, as defined in table A. The selection can also be made on the basis of any other marker linked to the genetic determinants and representative of the presence of these genetic determinants by opposition to the resident sequences of the susceptible parent.

A method or process as defined above may advantageously comprise backcrossing steps, with other elite lines, preferably after step c), in order to obtain plants having all the characterizing features of commercial S. lycopersicum plants.

The plant used in step a), namely the plant corresponding to the deposited seeds can be a plant grown from the deposited seeds; it may alternatively be any plant according to the 1^{st} aspect of the invention, bearing a QTL11, conferring the phenotype, preferably bearing these sequences homozygously.

The plant selected at the end of the process is preferably a commercial plant, especially a plant having fruits which weight at least 10 g but preferably 25 g, at least 100 g, at least 150 g or at least 200 g at full maturity in normal culture conditions.

The selection of the progeny having the desired improved phenotype can also be made on the basis of the comparison of the ToBRFV resistance from the S. lycopersicum parents, through protocols as disclosed inter alia in the examples, or on the basis of ToBRFV replication.

The invention also concerns a method for conferring improved ToBRFV resistance or for improving the ToBRFV resistance of a S. lycopersicum plant, comprising a QTL11 as defined, or the Tm-1 gene, by genetically modifying said plant to introduce a resistance gene, coding for a resistance protein having at least 90% sequence identity with SEQ ID NO.1. The genetic modification can be carried out by any methods or means well known to the skilled person. The resistance gene is preferably introduced into chromosome 8.

The present invention is also directed to a S. lycopersicum plant and seed obtained or obtainable by any of the methods and processes disclosed above. Such a plant is indeed a S. lycopersicum plant having the improved phenotype according to the first aspect of the invention, due to the presence of the combination of genetic determinants of the invention in its genome.

In view of the ability of the resistant plants of the invention to restrict the damages caused by ToBRFV infection, they are advantageously grown in an environment infested or likely to be infested or infected by ToBRFV; in these conditions, the resistant plants of the invention produce more marketable tomatoes than susceptible plants, or than plants comprising only one of the genetic determinants. The invention is thus also directed to a method for improving the yield of tomato plants in an environment infested by ToBRFV comprising growing tomato plants comprising in their genome the combination of genetic determinants, as defined according to the previous aspects of the invention, and conferring to said plants improved resistance to ToBRFV.

Preferably, the method comprises a first step of choosing or selecting a tomato plant comprising the combination of genetic determinants of interest. The method can also be defined as a method of increasing the productivity of a tomato field, tunnel, plastic house, greenhouse or glasshouse, or as a method of reducing the intensity or number of chemical or fungicide applications in the production of tomatoes.

The invention is also directed to a method for reducing the loss on tomato production in condition of ToBRFV infestation or infection, comprising growing a tomato plant as defined above.

These methods are particularly valuable for a population of tomato plants, either in a field, in tunnels or in glasshouses.

Alternatively, said methods for improving the yield or reducing the loss on tomato production may comprise a first step of identifying tomato plants resistant/tolerant to ToBRFV and comprising in their genome the combination of genetic determinants of the invention, that confers to said plants improved ToBRFV resistance, and then growing said resistant plants in an environment infested or likely to be infested by the virus.

The resistant plants of the invention are also able to restrict the growth or multiplication of ToBRFV, thus limiting the infection of further plants and the propagation of the virus. Accordingly, the invention is also directed to a method of protecting a field, tunnel, plastic house, greenhouse or glasshouse, or any other type of plantation, from ToBRFV infection, or of at least limiting the level of infection by ToBRFV of said field, tunnel, plastic house, greenhouse or glasshouse, or of limiting the spread of ToBRFV in a field, tunnel, plastic house, greenhouse or glasshouse, especially in a tomato field. Such a method preferably comprises the step of growing a resistant plant of the invention, i.e. a plant comprising in its genotype the resistance QTL on chromosome 8, or a gene coding for a resistance protein having at least 90% sequence identity with SEQ ID NO.1, as defined and a QTL11 on chromosome 11 or a Tm-1 gene, conferring to said plant improved ToBRFV resistance.

The plant of the invention to be used as mentioned above preferably also comprises the Tm-1 gene and/or a QTL9 on chromosome 9.

The invention also concerns the use of a plant resistant to ToBRFV for controlling ToBRFV infection or infestation in a field, tunnel, plastic house, greenhouse or glasshouse, or other plantation; such a plant is a plant of the invention, comprising in its genome the combination of genetic determinants as defined above.

This use or method is also a method for disinfecting a field, tunnel, plastic house, greenhouse or glasshouse by decreasing its viral population.

All the preferred features of the combination of genetic determinants are as defined in connection with the other aspects of the invention. According to an embodiment, the plant comprises a QTL on chromosome 11 which is obtainable or transferable from a plant of the seeds LVSTBRFVRES2 NCIMB accession number 43591 and is identifiable by at least of one of the markers TO-0201237, TO-0201238, TO-0201239, TO-0201240, TO-0201241, SL2.50ch11_9684449, SL2.50ch11_9779896, SL2.50ch11_9823405 and SL2.50ch11_9924232, especially at least one of allele G of TO-0201237, allele A of TO-0201238, allele A of TO-0201239, allele A of TO-0201240, allele A of TO-0201241, allele CT of SL2.50ch11_9684449, allele AT of SL2.50ch11_9779896, allele C of SL2.50ch11_9823405 and/or allele GT of SL2.50ch11_9924232. According to another embodiment, the plant comprises a QTL on chromosome 11 which is obtainable or transferable from a plant of the seeds HAZTBRFVRES2 NCIMB accession number 42758 and is identifiable by at least of one of the markers TO-0122252, TO-0144317, TO-0142270, TO-0142294, TO-0142303, TO-0142306, TO-0181040, TO-0123057, TO-0125528, TO-0162432 and TO-0162427, especially at least one of allele T of TO-0122252, allele C of TO-0144317, allele T of TO-0142270, allele G of TO-0142294, allele A of TO-0142303, allele A of TO-0142306, allele G of TO-0181040, allele G of TO-0123057, allele A of TO-0125528, allele C of TO-0162432 and allele T of TO-0162427.

According to another independent preferred embodiment, the resistance QTL on chromosome 8 is obtainable or transferable from a S. lycopersicum plant grown from seeds of LVSTBRFVRES3, representative seeds of which were deposited with the NCIMB under accession number NCIMB 44125, by crossing and selection of resistant plants. Such a QTL is advantageously characterized or identifiable by at least one of: allele G of SNP marker TO-0202968, allele A of TO-0202572, allele A of SNP marker TO-0202970, allele A of SNP marker TO-0202972 and allele A of SNP marker TO-0202974.

In still a further aspect, the invention also relates to a method of producing tomatoes comprising:
a) growing a S. lycopersicum plant of the invention, comprising the combination of genetic determinants as defined previously;
b) allowing said plant to set fruit; and
c) harvesting fruit of said plant, preferably at pre-mature or mature stage.

All the preferred embodiments regarding the combination of genetic determinant are already disclosed in the context of the previous aspects of the invention. The method may advantageously comprise a further step of processing said tomatoes into a tomato processed food.

According to still another aspect, the invention is also directed to a method for obtaining transgenic S. lycopersicum plants having improved resistance to ToBRFV, comprising:
Obtaining a construct comprising a resistance gene coding for a resistance protein having at least 90% sequence identity with SEQ ID NO.1,
Introducing said construct into a S. lycopersicum cell, comprising in its genome at least one QTL on chromosome 11, conferring resistance to ToBRFV when present homozygously, especially foliar resistance, or tolerance, or into a S. lycopersicum cell, comprising in its genome a Tm-1 gene, homozygously or heterozygously,
Regenerating a transgenic plant;
Optionally propagating the obtained plant.

According to a preferred embodiment, the resistance gene codes for a resistance protein which restricts the viral replication. Preferably, said resistance protein has at least 95% sequence identity with SEQ ID NO.1, preferably at least 96% or even at least 97% or 98% sequence identity. The resistance protein may also have SEQ ID NO.1, or may comprise SEQ ID NO.1 but be longer.

The construct may be an integrative vector introducing the resistance gene into the genome, preferably the nuclear genome of the plant. In such a case, according to a preferred embodiment, the construct targets the integration of the resistance gene into chromosome 8.

Alternatively, the construct may also be a non integrative vector.

According to yet another aspect, the present invention is directed to a method of producing a S. lycopersicum plant having an improved resistance to ToBRFV, comprising:
obtaining a part of a plant according to the first aspect of the invention, comprising the combination of genetic determinants as defined,
vegetatively propagating said plant part to generate a plant from said plant part.

According to another, distinct, aspect, the present invention is also directed to a plant, seed or cell of Solanum lycopersicum resistant to Tomato Brown Rugose Fruit virus (ToBRFV) comprising in its genome or genotype the combination of:
i) a resistance quantitative trait locus (QTL) on chromosome 8 conferring resistance to ToBRFV, as defined above,
ii) the Tm-1 gene, as already defined,
   wherein said combination provides ToBRFV resistance against a wider range of ToBRFV viral strains than provides QTL8 alone, or provides better protection against ToBRFV in a context of co-infection by ToBRFV and one or more additional tomato viruses, especially in a context of co-infection by at least ToBRFV and Pepino Mosaic Virus. Such a resistance is not provided by Tm-1 gene alone, which does not provide resistance against ToBRFV or against Pepino Mosaic Virus, but which is expected to potentiate the resistance provided by QTL8. The combination inter alia provides a better resistance against leaf symptoms, with less viral replication, especially at high temperatures.

The genetic elements i) and ii) are as defined in the preceding sections of this description, inter alia the QTL8 is preferably obtainable from the deposited seed NCIMB44125, by crossing and selection, or from the consensus gene as described previously.

The QTL8 and Tm-1 gene can be present independently homozygously or heterozygously; according to specific embodiments, they are both present heterozygously, or QTL8 is present homozygously and the Tm-1 gene is present heterozygously, in the genome of a plant, seed or cell of the invention according to this aspect.

The ToBRFV resistance against a wider range of viral strains means a resistance against at least two different strains, recognized as different (different infectivity, different symptoms, different temperatures, different hosts, and/or different aggressivity, etc. . . . ), generally isolated from different locations. This is the case for the ToBRFV strains isolated in Israel (Luria et al) and Jordan (Salem et al). Preferably, the combination (i) and (ii) provides resistance against at least 3 different ToBRFV strains, preferably against strains having a different infectivity or a different aggressivity, and/or isolated from different geographical area. According to an embodiment, the combination provides ToBRFV resistance against strains isolated from Israel and Jordan at least.

According to another embodiment, the resistance provided by the combination is very effective in case of combined infections, i.e. simultaneous infections by ToBRFV and one or more additional distinct virus. Such combined infections are indeed known to be dramatic to tomato plants susceptible to ToBRFV, which die from these multiple infections including ToBRFV, whereas none of the infections, taken in isolation, leads to death of the plants.

The additional virus is e.g. Pepino Mosaic Virus, which is dramatic for tomato plants especially in case of co-infection with ToBRFV. A third opportunistic virus may also be infecting the plants. In such a case of multiple infections, tomato plants bearing the combination of genetic determinants as disclosed is more resistant than plants comprising only the QTL8 or only one QTL reported in the art as providing ToBRFV resistance.

The invention is particularly directed to seed comprising this combination.

All the preferred embodiments disclosed in the context of the other combination of the invention are entirely applicable to this combination, including the markers for detecting the presence of these determinants, the preferred sequences or embodiments of the QTL8, and the preferred additional sequences which can be present in the genome of the plant, cell or seed.

The present invention also relates to methods for detecting the presence of said combination in the genome of a S. lycopersicum plant, cell or seed, based on the markers already disclosed in connection with other aspects, especially the alleles associated to resistance.

The invention is also directed to different methods for conferring the improved resistance, against a wider range of strains, or in a context of co-infections or combined infections, to S. lycopersicum plants, comprising introgressing or inserting the QTL8 as defined, and introgressing or inserting the Tm-1 gene, unless the plant already comprises the Tm-1 gene; in this last case, only the QTL8 or corresponding consensus gene is to be incorporated.

The present invention is also directed to methods for improving the yield of tomato plants in an environment infested by ToBRFV or by multiple viruses including ToBRFV, comprising growing plants comprising the combination as defined in this aspect of the invention.

The present invention is also directed to methods for reducing the loss on tomato production in condition of ToBRFV infestation, or in condition of multiple viruses including ToBRFV infestation, including at high temperatures, comprising growing plants comprising the combination as defined in this aspect of the invention.

The different steps of these methods, or different uses of the combination as described according to this aspect, are identical to those described in conjunction with the other combination of genetic determinants of the invention.

The invention also encompasses different selection methods, especially methods for selecting S. lycopersicum plants having ToBRFV resistance, inter alia a high level of ToBRFV resistance, comprising assaying the plants for the presence in their genome of a combination of genetic determinants according to the invention. A combination of genetic determinants according to the invention is as defined previously, namely a resistance gene or QTL on chromosome 8, and either a resistance QTL on chromosome 11, or the Tm-1 gene. The step of assaying the plants for the presence of this combination can be carried out by any means known to the skilled reader, and preferably by the use of markers, namely at least one marker per genetic determinant. Suitable markers for the resistance gene or QTL on chromosome 8, and for either QTL on chromosome 11, or Tm-1 gene, have already been mentioned in the description.

The plants comprising the combination of genetic determinants are preferably selected.

According to still another aspect, the invention is also concerned with a polypeptide or protein comprising an LRR (Leucine-rich repeats) domain, preferably a NBS-LRR protein, wherein said polypeptide or protein has at least 96% sequence identity with SEQ ID NO.1 and provides resistance to ToBRFV to tomato plants. The percentage of sequence identity can be measured by any means, but it is preferably defined by the Smith-Waterman algorithm. Preferably, the percentage of sequence identity with SEQ ID NO.1 is at least 97% and even preferably 98% or more, especially as defined by the Smith-Waterman algorithm.

According to a preferred embodiment, the polypeptide or protein has at least 99% sequence identity with SEQ ID NO.1, for example 100% sequence identity, thus the polypeptide has SEQ ID NO.1.

The differences in the sequence of the polypeptide with respect to SEQ ID NO.1 are preferably not in the LRR domains.

The polypeptide or protein as defined above is capable of restricting the viral replication of ToBRFV in infected plants expressing said polypeptide or protein. Preferably, said capacity of restricting the viral replication is irrespective of the strain of ToBRFV infecting the plant.

The invention also concerns a resistance gene encoding a polypeptide or protein as defined above, conferring resistance to ToBRFV infection in tomato plants. In some embodiments, the resistance gene has a sequence having at least 80% sequence identity with SEQ ID NO.2. in view of the degeneracy of the genetic code, the sequence of the resistance gene may differ significatively from SEQ ID NO.2 however still encoding a protein or polypeptide of the invention as defined.

In this respect, the invention also encompasses gene constructs and vector, comprising a resistance gene as defined above, coding for a protein or polypeptide having at least 96% sequence identity with SEQ ID NO.1.

In view of the advantageous properties of the resistance gene as defined, especially the high capacity to restrict viral replication, the present invention is also directed to a plant, preferably a S. lycopersicum plant, resistant against ToBRFV, comprising in its genome, preferably on chromosome 8, a resistance gene coding for a protein or polypeptide having at least 96% sequence identity with SEQ ID NO.1.

Said resistance gene can be present homozygously or heterozygously in such a plant. This specific resistance gene can be combined with other genetic determinants, for example with a QTL on chromosome 11 as defined in previous sections of the invention, or with the resistance Tm-1 gene, or both.

The sequence of the resistance gene as defined in this aspect of the invention can be used for conferring resistance against ToBRFV to a S. lycopersicum plant or for obtaining transgenic S. lycopersicum plants resistant to ToBRFV.

TABLE A
SNP markers useful in the invention, their position in the SL2.40 or SL2.50 genome assembly,
their flanking sequences, and the alleles of the SNPs linked to susceptibility (S) and
resistance (R), respectively

| | | | | |
|---|---|---|---|---|
| SEQ | SNP name | | | |
| ID | Position | sequence | S | R |

QTL11 on chromosome 11

| | | | | |
|---|---|---|---|---|
| 3 | TO-0201237 | GTTACTTTAATATTTAATTGTATTATAATAAATTCTATATACTAAGGG | A | G |
| | SL2.50: | ATGCAGTTATTTTATAATTTTGAGAAAATACATGGCATATTCGTTCA | | |
| | 8744810 | TCTTACTTGTGACCTTTTTTTTAATAGATTATAGTACATTCCTTATTA | | |
| | | AATGGGTTAAAGCTATGATCTGGAAATCTCCATGCTTAACGTCTGT | | |
| | | ATGTTTTCAAT[A/G]CTATGGTCAAACAATTTGCTCATATAATTTGT | | |
| | | GTACGTGTTATGAATTTGGGATTTTTCATATGAGTAAAATAGTTTTT | | |
| | | AAAAAAAATTCTGTTATGTCCTTGTGGGCATAGGTTCAACCTTGAA | | |
| | | GTAAATTTAGGGATCATAATAATGGGTGTTGTTAATTTTGAAATCTT | | |
| | | ATTATGGTCATTTGTTTGAGTAGATTGC | | |
| 4 | SL2.50ch11 | GCAAAAAAATAAAAAATAAAAAAACGCTAGACAGATTGATGACTAT | G | GT |
| | 9924232 | TTATGCTCATTTCAAAACATAACTAATTTGTATTTAATTTTTTTTTAC | | |
| | SL2.50: | CTTTTTTGATTATTTTTTTTGCTGTATGATTTTCTCGGTTCTATTATT | | |
| | 9924233 | AGTTATTT[G/GT]TTTTTTTAAATATTAATCATAATAACATAAATACAT | | |
| | | AATTAATCCCTTAACAAAATAAAATAATGACATTTTTAATAAACAAT | | |
| | | AAAATTGTTGCTAAGAAGTCCCACTAAAAGCTTAAATATTGTTGTTT | | |
| | | TGAAAAAAATTTCCCCAAGG | | |
| 5 | TO-0201241 | TCATCGTAGTCAGTAAGATTTTACCAAGGAATTAAAATTTATTAAGT | C | A |
| | SL2.50: | ATTAAGCTCATTACATTAGAATTAGCACCAAGATTTCCAAAAAGAA | | |
| | 9550430 | CTAAATTGGAATTAGCAAAAATCTGAAATTTGAAGAGTTAAGTTAA | | |
| | | GTATGAGTTTTGAGTCAACTTCAAAGGACCATAAATATTAGAACAC | | |
| | | GATGAGTTAGATGTG[C/A]TACAAGATACCATAGGGAAGATATTCG | | |
| | | AATAACCTTTGCAAAGCCTTTGAGTTTACTAAGTTTCAAGTTCATA | | |
| | | CAAGTGAGATATGACCTTTTGAAGTTAGGTTCTCCAGTTAAGCAAA | | |
| | | GTTAGCCAAAAATAGTGAAGGTATTTTGGTCCTTCCCTTACCCAAA | | |
| | | CAGATTTAATTCGTTTTAGTCATATTTTAGGGTTA | | |
| 6 | TO-0122252 | ATGGCAATAGTGAACTGCAGATACAACTGAAATTGCAGAACACCC | A | T |
| | SL2.40/2.50: | TTAAA[A/T]ATAGAATCAATAGAAAGTTGCAACAATATTTGAATGAT | | |
| | 8090264 | GAAGCAACAAAG | | |
| 7 | TO-0162427 | GCACCAGTTATAGTAATGTCCTGCTTCTTTCCTGTACCCTTATCAG | C | T |
| | SL2.40/2.50: | TAGC[C/T]GTGACAGAAAGAATACCGTTGGTGTCAATGTCGAACTT | | |
| | 10018811 | CACTTCAATCTG | | |
| 8 | TO-0144317 | AGCCATTGTGATTGTGTCTGTTGTACATTACCAAAATTCTCTAGAG | T | C |
| | SL2.40/2.50: | AAAG[T/C]GATACACATGCCAGCCCTATCGATATAAAGCAACGCAA | | |
| | 8334467 | GGTGGATTCTGC | | |
| 9 | TO-0142270 | AACACCAGGTAGAGAGCACAGCGAAACAATGGCCTCAGGAAGAT | C | T |
| | SL2.40/2.50: | CTACTT[C/T]GCGAAGTGCAGCAAGCCACTCCATACCTCCACCAG | | |
| | 8633469 | GCTTTGATTTCAGTG | | |
| 10 | TO-0142294 | TCAACTGCAACTTTAACAGCTGATTCAACTTCTTCTTCTTTCGAAA | A | G |
| | SL2.40/2.50: | CATC[A/G]CATTGAATGTAACGACCTCCAATAGATTCAGCTAAACT | | |
| | 8764030 | TGTACCTACTTC | | |
| 11 | TO-0142303 | GAGGAGCTATCAACTTCATAGTCAGATTCAGAAAATGATTCAGATG | C | A |
| | SL2.40/2.50: | AGGA[C/A]GTGGCTGATTCTTCTTGTTTTCTTTTCTTCCTTCTGCTC | | |
| | 8903092 | GAACTCTCTCC | | |
| 12 | TO-0142306 | CAGAAATAATAGAAAATCAGAAAGAAAAATCAGCTTTCTAAATGGA | G | A |
| | SL2.40/2.50: | AAAG[G/A]CGATGGCACTATGTTTGAAGTTTTAAGCAACTTTTCTG | | |
| | 9318832 | AAGTCCCAAAAG | | |
| 14 | TO-0181040 | CTCTTGGTGACAAACCACTGGCTCAATTTCTTCGCGAAGCTAAAG | A | G |
| | SL2.40/2.50: | CTATC[A/G]CTGATGAGCTTGTCACGGCAGGCACACGTGTCTCCT | | |
| | 9797143 | GATGAATTCAATGC | | |
| 15 | TO-0123057 | CATTACTGTTGAGATATCTCATCGGCAACCCCTGGAGCTTGCCCA | T | G |
| | SL2.40/2.50: | CCCGC[T/G]TGTCCTCCAGGATCTGATTTCAGAAAGGATGAATAGT | | |
| | 9825111 | AACTGTGTTTCAG | | |
| 16 | TO-0125528 | CAAGAACCCAACGACTTCTTCTTCTTTGCTTATTGAAAAACTTGGT | G | A |
| | SL2.40/2.50: | TTTGAAATGAAAGG[G/A]ATCGAGAAATTGGATACTCAGTGGTTCT | | |
| | 9837711 | CTACTACTAAACCTTCTCCTGATTTTAAGAAA | | |
| 17 | TO-0162432 | TGATCGACAATTCTTGTTGTTGTTGAAACTCTGCAAGTGAGAGAG | T | C |
| | SL2.40/2.50: | GGATG[T/C]ATATAGAGAAAGGATATTGGTAAAGGACAATTCTAGA | | |
| | 10015478 | AGGGTCTAGGGAA | | |
| 18 | TO-0201238 | TGCAACTTCTATTCTTCCTTCCGCAGGTGGGAGCTGTGTATTTTAT | T | A |
| | SL2.50: | TTATCTTGTCTAGTTGTACTCTTATTAGCTTTTGTACTTGTTTAACT | | |
| | 8972905 | AGATCCATGAGAGTTGATAAATTACTTTACAAGTTTTAAGTAAATGT | | |
| | | GTGTCTAAAGATATTATTTATAAAATCATATTATTGCAATTACTTCT | | |
| | | GTTTCCTTAAAAA[T/A]TTTCACATGTTTAATTATTGGGATACGAGA | | |
| | | GTTCTCTTACTTAGGTGTTAGAGTGGTGCCTGGACGGCTCGGTGG | | |
| | | GTTGGATCGTGACATAATATATGAATTTGTCAAAAAAATTTACATTA | | |
| | | TTTTTGTGATCGCTATTTCAATATAATATACAGTAAGCATGCATATA | | |
| | | TGTACAATGGATATATTTTTGTCATGCTCTA | | |
| 19 | TO-0201239 | TGCGAGGCACTCGCCTTGATGGTTCTTACTACACTTGGGGCAAGT | C | A |
| | SL2.50: | GGGGTAAGTCTTGGTGCTTGAAACACTTCCCTAAGACTTAGATTC | | |
| | 9290095 | TGGTGCCCTAACCTTCTGATCATACATGTTCTTAGAGGATGGAATA | | |
| | | CTAGTTATCATAGTTCTGCTATGGGGTTGAAAACTTTTCCTGACTC | | |
| | | TACGAGCGATTTCCACCA[C/A]CAAATTTCTGCTAAGAGTAGTCAT | | |
| | | AGTTCCCAGTCCTAACCTCTTATTCTCCTTGGCTTGTTTCCTAAGC | | |
| | | TTATCACCCTCAACCTGCTGATCATGAGTCATAAGCTTAAGGATGT | | |
| | | TCATATCTCGTAGTAACATAGAACTTCTGCACTCAGTTTTCACCAA | | |
| | | ATTTTGACTCCGTACAAGAAATTATTCATCTGAGCCCT | | |
| 20 | TO-0201240 | AGTACAAATTGCAACTTTTTCAATGTTAACGTCTGCTTTTTTAGTGT | C | A |
| | SL2.50: | ATAAAAAAGAAGGATTCATGAACTCCTCAGTCATATTCTATTTTAAA | | |
| | 9461743 | ATACTTAAACATCTTTTTTAATTATGTCTCAAGCATGTCAAACATCT | | |
| | | AATGCAAAAAAGACTTACGTTTGTCGTTGTGGTAATTTAGCTATTT | | |
| | | TGAGGATTTCACA[C/A]ACCGATACGAACCCAGATCGATAATTATT | | |
| | | TAATTGTGCAATTGGTGCATTGATCTTTTTTTAAGTGGCTTGATTAT | | |
| | | GATTCATCAACGAGCAACCCATCAAAATTGAGTCTCGGATTCGAG | | |
| | | ACATCAAATTTTCAAAGCGTTATCAAATTTCAAATATTCTTTATCTC | | |
| | | CTTATTTTATCTCATGTACAACTTGATTGACT | | |
| 21 | SL2.50ch11_ | AGTGATATTGTCTTCAGCTATAGAATATTATCCAGCCCCTCTAGCT | C | CT |
| | 9684449 | TTGGTCGATTTTTTTTTATAACAAATTTTATCCAAGCTATCATTGTTT | | |
| | SL2.50: | TTTTTTTTTTTTCAATTTACAGCAATTGTTTCCCGACTAGCCTATCA | | |
| | 9684450 | ATGTTAGAG[C/CT]TTTTTTTTGGTTTGAACATCGTTGTCTTGAGAA | | |
| | | GCTTAATCCATGTTCACTGTAGGCATTGTTATTTAATTTTCAATTGA | | |
| | | GTTTGTTAATCTGCCTATAGATATTAATTTATTTCCTCTTCTTTCGA | | |
| | | TGCATATGATCAATCCAGTCTAT | | |
| 22 | SL2.50ch11_ | ACAATGACATGACATCAAAAATAATTTAATTTATCATGTCAAATTTG | A | AT |
| | 9779896 | TTTAAAAATAAATTGTAATCGAGTGATTTTAAAGAATAAAGATGTTA | | |
| | SL2.50: | AGTTGAGTGATTTTAAAGACACAAACTAAAATTTAGTGACTTTACA | | |
| | 9779897 | AGATAATTTC[A/AT]TATAGTTCATTGACCTTTTAAGATATTAACTCA | | |
| | | AAAAATCAATAAGTAGTCACACACAAAAGGTGATTGCAAAGTAAAT | | |
| | | TTCAATTTTTAGCATAAGCTTAGTGGGGAATACCAATGACGAATCT | | |
| | | AAAAGTATCAATACAAAAAATAGAA | | |
| 23 | SL2.50ch11_ | AAATGTTAATTTTTTTATTTATAATAAATATTAATATGTCTTAGACTT | T | C |
| | 9823405 | GAACTTTTAAGTTCATTAAAGATAGTTAAATATCGTATTTGATATAC | | |
| | SL2.50: | TTAACGAAAAAGCTAAAACGTAAAAGAAAATATAATAAGTTTATATC | | |
| | 9823405 | TTCTTCTT[T/C]TTTTTTTAAATTTGGATTTGTGATAGTAGGGCTTTG | | |
| | | AGAGAAAATTCTTTTTATAATTTTTTTTTTCATATTCAAGGCTCGAA | | |
| | | CCTAAGATCTATGATTAAGGAAGAAGTAGCCTTTCCACTATACAAT | | |
| | | ACACGTTCATAATGAGTTAAT | | |

Tm-1 on chromosome 2

| | | | | |
|---|---|---|---|---|
| 31 | TO-0178073 | GCCCTGCCCCCGACACCCGTGGCATGGAACACCAAAGTCTCATA | G | A |
| | SL2.50 | TCCTTCTTTCACTAATCTTTCTTTGACAGCATTAACACAAGGAGTC | | |
| | 34284984 | GTAACCCCAAACATAGTTACACCAACTGTAAACTTTCCATTAGTGA | | |
| | | TGCTATGCTCTTTTGAACTTTCAAGCCTNCCNATCACCATTCCAGC | | |
| | | AAATGCTGCACCCGCATTAGATAGAACAACCTTACTNACATTGTTA | | |
| | | ATCCCACAAATATCTACAACTGAAGGAAACAATACCAAGTCTGATG | | |
| | | TTCCAATATAAGATTCAGTTTG[A/G]CCACTGGCAACTGTAGATATT | | |
| | | ATAACTTTTGGGATCCCAATTGGAAGAGATCGGAAGGCAGATGAC | | |
| | | AATAGAGATGTTCCTCCACTACCCCCA | | |

Resistance QTL on chromosome 8

| | | | | |
|---|---|---|---|---|
| 39 | TO-0039023 | ATCAAAACACATTCTTTAATTCTCTAGAAAATAATATGAAGGTGGG | G | C |
| | SL2.50 | ATGTGGTGGAGAGT[C/G]GGGAGTGAGGTTTCGTTGAGCTCTTGC | | |
| | 53643714 | AATGAGGATCTTGTGTTTCAGTATGGTTCTTGA | | |
| 40 | TO-0202966 | ATTAGACATGGGAAACAGATCCATTCACACCTCATCAGATCTGGTT | A | G |
| | SL2.50 | TTTACAAAAATGCTTTTGTATCAGCTGCTCTTGTGGATATGTATAG | | |
| | 59480063 | TAGCTGTGGCAGGTTAGACATTGCTCTTCAGGTATTCCAGAGTTC | | |
| | | AGCCGAGAGATCT[A/G]TTGCAGCTTGGAATTCAATGATCTCTGCA | | |
| | | TATGGTTTCCACAGCAATGGTCAGAAAGCAATTGACATCTTTCATG | | |
| | | AGATGATTAATTCGGGACTTACTCCGTCTAAAGTCACATTTATCAA | | |
| | | CTTATTAACAGCTTGCAGCCACACAGGGC | | |
| 35 | TO-0202968 | GTGAAATAATCATAGAAAAAATCTGGTTTTGGTTGGTAGGTTGACC | A | G |
| | SL2.50 | TCTAGTCATGATTGGTACTAACAGTTTATTCTGAGATGTCTGTGAC | | |
| | 59615952 | ACTTTAGATCTTGGGCTAGCTTGAAGCGTTGTGTGGAAATCCTTG | | |
| | | AGCTTGTCAGKGT[A/G]TGTGTGTATTACTATAAAATGTATGCAGA | | |
| | | GTTGAGTAAGTAAAGAAATTTTGCAGGCTCCAGTTTTGAGAGAATA | | |
| | | CCCGGCAAGCTTAACATTGACAGCATATTCTTACCTTTTTGGCGTT | | |
| | | ATACCAATGATAGGGACGTCATTCCTTGT | | |
| 32 | TO-0202572 | TCCCCGTGCTTGAAGAGTTGACAATTAGGTGTTGTCCTGTGTTTG | C | A |
| | SL2.50 | TTATTCCGACCCTTTCTTCTGTCAAGAAATTGGTAGTTTATGGGGA | | |
| | 59 780 025 | CAAGTCAGATGCAACAGTTTTGAGGTCCATATATAATCTTAGGGCT | | |
| | | CTTACTTCCCTCA[C/A]CATTAGCCTTAACTCCATAGCTACTTCGCT | | |
| | | CCCAGAAGAGATGTTCAAAAGCCTTGCAAATTCTCAAATACTTGGA | | |
| | | AATCTCTTTCTTCGACAATCTCAAAGAGCTGCCAAACAGCCTGGC | | |
| | | TAGTCTCAATGCTTTGAAGCATCTGGAGA | | |
| 36 | TO-0202970 | CCCCAGCCATTGGTACTCCAAAGTGAAGCTCGTACATTGCAGGAA | C | A |
| | SL2.50 | TATTTGGAGCCAAGGCAGCAACCTGCTCAGAAATTTAGCTAAACA | | |
| | 59910728 | TAAGAGCAGCACCAATATAACAGACAGTGATCTGAATAAGAACTA | | |
| | | ATTTCCTTCCTGCTA[C/A]AAATATAAAAACACCTAACTAAGTGATA | | |
| | | GAGGAGGCAAATGCTTAATTGATTCCATGATATGTTATACTTTCAA | | |
| | | CATACTTGTGTTTCATGACTCATCACTGACATCTAAGTCTTAGTGC | | |
| | | CACATCTTTTATGTTTTTTTTTAACAAACC | | |
| 37 | TO-0202972 | GGCACGCATGTTGACATCAAAGAAGACAGTATTGTGGTTTGCATA | G | A |
| | SL2.50 | ACGGTCAGAAGGATCAATTCCCTCAAAACGTTGAGGAAACTGAAC | | |
| | 60288570 | ATAACAGATCCTATCTCCACCGCGGTCCATCATAAAGCACATTCC | | |
| | | CTCCCTGATTGCCTC[G/A]GAGTTGTAGATGTAGTGATCACAATCA | | |
| | | AGGTTGAGGATAAAGGGGCCATTAGACATGATGGCAGAAGCTCT | | |
| | | GACCAAGGCATTCATGGCACCAGCCTTTTTGTTATGATCATAGCC | | |
| | | AGGACGCTTYTCACGAGAGACATACACCAGCAAG | | |
| 38 | TO-0202974 | TGTCATGTCTCTGAGAAGTGAACAAGCAGAGTCTACCTCATTGAC | G | A |
| | SL2.50 | TATACATAATGCTTGTATCACCCGAGCAAAAGTGAATACAGAAGG | | |
| | 60612041 | AGAAATACCTTTCCCCAACATTTCATAAAATACATTTGGAGCAACT | | |
| | | TTGGGACAATTTCY[G/A]GCTAACAGAATATCCAACGCTTGATTGT | | |
| | | ATGACTTGAAAGTGGGTTCACAAGAGAAAGTATTCCACATATCCAA | | |
| | | AAGTATTCTAGTTGCTTGCCCTGGTAAACCAGCTCTTCCATAATGC | | |
| | | CTCATTATCATAATAAAGAGGGACTCTTTG | | |
| 41 | TO-0202975 | TATGTTGAATTTTGGTGCTCCTGCCACAGGGGAGAGCCCTCCTTC | A | G |
| | SL2.50 | TCAAGGTGATGCCTCAAGTGACTCGTCTGAGGAAAATGGCGACA | | |
| | 60971123 | GTCCTTTCCAACATGAATCAGGACCTTATGGTAACGCTGGCCAAC | | |
| | | CAATGCATGGTATGTC[A/G]ATGTATGCAAATATGGGCTGGCCAAA | | |
| | | ATCGCTGTGAAAACACTTTTTCTGTGTCGACATGAAGTTGCCAAG | | |
| | | GGTWTTCCCCTTGCCAACGCTAACAGGGCAGAGTATAGGCGCGT | | |
| | | TTCTTGATTTCTCAACTTGAGCTTATTTTCCTTAT | | |

QTL9 on chromosome 9

| | | | | |
|---|---|---|---|---|
| 42 | TO-0180955 | TTCCGAAATGAGGACGATCCATCAGCTTCTTCAGCTGAGAGCCCC | A | G |
| | SL2.40/2.50 | TGGTC[A/G]ACATACCAGAATTCTGTTTTTCTAAAACTGTCCAAAAT | | |
| | 4800680 | CTCCTGTAAAGA | | |
| 43 | TO-0196724 | GATTTGAATGCCTTGCCACAGCCAGAGGATGACGA[T/C]GAGATTT | T | C |
| | SL2.40/2.50 | TTGGACAACAATTAGAAGATGAACCACA | | |
| | 5203457 | | | |
| 44 | TO-0145125 | AGAGAATGATATCACTGCCTTAGTTTCTCAATTAAAAGTTGTGCA | A | G |
| | SL2.40 | AAAACAAAACACACA[A/G]CTAGATGAAGAAAACAGAGCATTCGC | | |
| | 40025769 | CTCAAAGCTTCAGACAAAAGAAGTTGAGAACAAC | | |
| | SL2.50 | | | |
| | 44428188 | | | |
| 45 | TO-0196109 | TACAATACCTTCTGGCATCCCTTTCCGCAAAACGA[T/G]AGATCTT | T | G |
| | SL2.40 | TAGTATCAAAACCGAGAGCACTGTCACC | | |
| | 59014540 | | | |
| | SL2.50 | | | |
| | 63417021 | | | |
| 46 | TO-0201220 | GTTGGGAGGCAGAAGGGTATAAGATTGGACACTGAAATTTTTGCT | A | G |
| | SL2.50 | ACTTACCGTAAGCTCATCAACATCGACTGTTAGAAACATCAGAGAA | | |
| | 11229842 | AGATATTTCTCAGACAGCTCACAGTAGAATGGAGAGATCATTCTAC | | |
| | | ATGGACCACACCATGAAGCACTGAAATTTGCAATAACCTGCCAAA | | |
| | | ATTTCACTTGCGTAAGAA[A/G]GGTCCAAAATGCCAAAGCCAGCAA | | |
| | | AAATGTAAAATTTTAAGCAAGCAAAAGGCGAGGGATCCATATGGG | | |
| | | GAAACTGCTGCACCATTCTGCTATTATCCTTCTCTTCGCATGTGTG | | |
| | | CAAAGCATTAGAGTTAAAGTGCCTTGAGTATTGTTTTTTAACGTCT | | |
| | | TTTGAATTTTAGTGCAGCTGGGCTAGAGGATTAAGTTAC | | |
| 47 | TO-0201221 | TACTGTACCAATATTCAGTGCAAGGTATAGGTGTCCATTCATGAG | A | G |
| | SL2.50 | GGCTGTCACCCATGAACACCGAAAATGCTGCTCACAGTACTTGCT | | |
| | 18086313 | GAGAGCTTCAGACGAACCATCCATTTGCAGATAATATCCAGTGCT | | |
| | | CAAGATAGGGGTGTTTGTTGGAGCCGGTGAATTTTTTCCAGAAGA | | |
| | | GAGTATATGTTTGGGATCAC[A/G]GTCTGTGACAATGACCTCGGG | | |
| | | ATGTCCATGTAGCCTAATAATTTCAGAAGCAAATGAAGAAGCCATA | | |
| | | GTTTGAGCTGTGAAGGTAGAAGAAAAGGCAATGATTGATTGCCCA | | |
| | | TATTTGAGTTACCAGTCAACCAACGTCAAGATTGTTACCTTCCCTT | | |
| | | CGACCCCAAAAGGCAAGTAATGCATTGGTACCTCCTTAAACA | | |
| 48 | TO-0201222 | TACTATCGAAGGGTAAAAATAAAAAACAAGTTGTTCAAATATCCAT | G | A |
| | SL2.50 | TAGAATGAAGAAAAGAAAAGCAAGTTAGGGGCTCTTGGACCGGGT | | |
| | 31451276 | GGTATCAATGTCTAAGCAATGGTTCTCCAGAGGCGTCACACCATT | | |
| | | CCAAGCCTTATTCAGCATGGAGAAAAATCCCTGAAATCTAATGGC | | |
| | | TCAAGTCTAGAAATAGCCT[G/A]GTCGATAGACACACAAGTAAAAA | | |
| | | TTACCTTTACCGTCTTCTACATCATTAGCATTAGGCGTTCTCTGAC | | |
| | | CACTCCCTTCCTCTTCTCCTTTTGCAAATGACGACAGAACATCTCG | | |
| | | ACTTCGAGGGAAGTTTTGGGTGGGATTGGAGTAAAATCGTGTATT | | |
| | | CACTCCCCATACTCAGCCTCCAAGTCCTATTTAACCTGTG | | |
| 49 | TO-0201223 | AAGTAGGAATACGGTCCATGGTCTGTGTTCATGGATTGAGACATA | G | A |
| | SL2.50 | ACTTACCCAATCACTTAAATGAACAACAGATGACCAGCGCGGAGC | | |
| | 29219682 | ATTGTTCAATCTATTGTCCGTATGTTTGATCATAACTGAGGTTAGC | | |
| | | AGTTAGTTACGGGAAATTTACGGATTGGATCAACTTAAAATTGTCA | | |
| | | TAAATTTTAGCACAAAAT[G/A]AATTAGGTTCTCCATAACCCATGAT | | |
| | | ATGATAGATAATTTAATATTCTCTCCATATCCACTGAGTTTGCTAAA | | |
| | | ATTTGACCTCCGAGTGAAAAGTTATGCCTATTTTAGTGAAGGCCTA | | |
| | | TTGAGTAGACCCTAACGACAGACCGTCTATTGAACAACGGCCCAT | | |
| | | CAGTCCAGGTCGTCGATTTCCGCGACAACAATTAGCT | | |
| 50 | TO-0201224 | AACAACCCAATATTCTTAGGAAATGATCCTTAAAATGGTGCATCTT | C | A |
| | SL2.50 | GCTTAGACAACCAATGTAAGGGCTACTTGACTAGAGAGAGAAGTC | | |
| | 27896493 | TCTGGGATGATTGAAGCTACCACAACATTTGGTTTAGCTCCCCTC | | |
| | | CATGTGTCTATTGATGATTTGACCGCTAGGGTCACAACTTGTGAG | | |
| | | AGCAAGAAAGGGGAGAAAT[C/A]TGAGGCTTTGTCTTTTAAAGACA | | |
| | | AGGTAGCAAAGCAGAAGAAGGACATAGACTATCCAAAGTCTACTG | | |
| | | ACTTCACTTCATTACTAGAGGCTGCTAATGATTTAGAAACAATTGA | | |
| | | TACTTTAGAGATTACTTCGCCTACCACCAGAGAGGTACATAGGGA | | |
| | | GGATGCTAGAATTGATGTATTAGATGCTGAAACTGATGAGG | | |
| 51 | TO-0201225 | CATTGCGAAATTGCATTTGTTCTCTTAATCAAAGTCTTGTAACTTTG | G | A |
| | SL2.50 | GGTGATTTAGTGTGTTCTCGGTGAACCGATCGGCAACTCACCGAC | | |
| | 22585892 | TACACATTTATGTCGCCGACTTGATTTTTCCCCCTCCCCTCCGGG | | |
| | | CTAGTATACTTGAACTGTAGGCGAATTGGGGAGCCACTCGGCAGT | | |
| | | TCACCAATTGGTTTTGGC[G/A]ATTACCAGGATCTTCTTTTTCTCCA | | |
| | | CTTGTTCAGCTCGTTTTTCTCCCTTTTTTCTAGAAGTGTCCTGCCT | | |
| | | TGCTTATTAACTCATATACCTTAAAATTAATGCTTTAACAATAGTTT | | |
| | | GTAGAATAAAATAGGCATTTAAGGACACTCAAACTACCAAAAAATA | | |
| | | TCCCTAAATGAGTAAAATCTTTGACTCATTATTTAT | | |
| 52 | TO-0201226 | GTAATAGGGTCATAAAATACTTGGTGTGAATTTACCAAACACCCTA | G | A |
| | SL2.50 | AGCATTGGCAGAAAACATATAGTGTTTGAACCCCTCAAAGACGAG | | |
| | 23241674 | ACCTAACAAATAGACCATCTGCGCATCAACGGACCGTTTGTGGGG | | |
| | | TCTCGTGGGTTGACACTTAGCTTTACATGGAGCCTGACTAACAAA | | |
| | | CCTTGAAGATCCAACAGAT[G/A]GAGCAAGAAGACAGGCCATCGG | | |
| | | TTCAACCATTGGCTGTCGTTGCCTTTGTTGGTGAACACCTCAAGG | | |
| | | AAATTTTTAACCAAGATTTGGGCTCTTTCTCTAGGGCCCCTTGTGG | | |
| | | GTCAAAGGTGGGGTCGTACTAAACAATTAAATTCCTTAACTATGTA | | |
| | | ATATAGGTTTAGGAATATCTCATGCAGGTTCCAATAAAAAA | | |
| 53 | TO-0201227 | TGAAATGTTTAAGGTTTAGATTGGTTGGTTCGCTCACATAGGAGG | A | C |
| | SL.50 | GTAAGTGTGGGTGCCAGCCCCAGCCCGGTTTTGGGTCGTGACAA | | |
| | 24571242 | TTGGACTTCCATCTGTGGTGATTTTAATACACCGATATGGGTTTTT | | |
| | | CCCATGCTATTATCTTCACTTTAGTTTCATGAGTGCAAGCATCCCC | | |
| | | TAGTTGAAAGAGACCTTTC[C/A]ACATTGAAGGCCATTTGATTTAA | | |
| | | GGAAATGTTACAGCCAACATTCTGTATGTATCCATAATAATAACAT | | |
| | | ATTTTACTCTACTCATGTCGGATGCTCTTCCTTAATATATGCTTGC | | |
| | | GCACCTATTCTTTCACAAGATCCAACATATTATGACATGTCTGGCT | | |
| | | TATAAGAGTAGTTTGTGTGCCCCATTTTGGTGTTTTGTT | | |
| 54 | TO-0201228 | GACTTGACTCTTAACTTTTAGTCAATTGAATTATGGATTCAAAGTTA | G | C |
| | SL2.50 | ATTATCTCATGTTTATGGATGATTTTAAGATTTTTGAGTGTACCTTA | | |
| | 33865929 | AAGTGTGGGAATAAACTAGAAAACATAGGTATGTTGCTAGGGTAC | | |
| | | TAGTACGAAATGAAGTGGGAAAAGATTGATGAATTGGTAACCCTG | | |
| | | GCGCATTAAGAGGTGC[G/C]GGCCGCAAGTGGGTAACATCAGATA | | |
| | | CTAGTGTAGGGTTTTTTGGCTGGTATGGAGCCCCAGATTCCAAAC | | |
| | | TTCAGCGCCCCTAATGAGGCGCTTTGGCTGGAACAGCGCCTTAG | | |
| | | TCCCTTGCCCAGTTGAATTCCAACTTTTCTTGCTCATTTTTCAACTC | | |
| | | TAAACCCCTAATTTCAACTTGATTCTTTACCCAAACACA | | |
| 55 | TO-0201229 | TATGAGAGAGAATTTTTTAAACCATCTAATCCATGAAAAGGCTTCA | G | A |
| | SL2.50 | CAGACATTAGATCAGTACAAGGACCGTCAATGGGGTTCGTCAACC | | |
| | 39681757 | TAAAACTACAGACAGTGTGATATTTTTATTTTTTTCCATTGTTAGCT | | |
| | | CGTCATCCTAAAAGTGTTTTGAGTGTGCCGTTAAGATATATTGGTA | | |
| | | CTAAAAATTTCTCCGC[G/A]GGTACTATGTCTCCCAAGAGGCAAGT | | |
| | | GGTTTATACAAAAAGGGGCAAATTCAAGTTAGTTGGCCCTTCTTCT | | |
| | | TGGTTAATTGATGAAGACCTAGACTAGAAGAGGGACCCAGCCTAC | | |
| | | ATTCCTTCGGACATAAAAACACCACCAACAAAACCCTAAACTACTA | | |
| | | AGAGCACCATTCTAATGAGGACCGCACACTGTTCGGC | | |
| 56 | TO-0201230 | AAAATTTAACCATCAAATCTTCATTAAACATAGACGTATGGGATCT | A | C |
| | SL2.50 | AATTCACCTTTGAGACCTCTTTCCTCAAAGGTTTTCTTCAAAGTATT | | |
| | 44268811 | TTCAAAAGTTGAGTTTTCTTGTAGGTGCATTCATCCGAATTTGGAC | | |
| | | CTCCCACAGTGGTTCAAAGTTCAAAAATTTGATGTCATTGGAGGAA | | |
| | | TAGGGAACCCCTTAG[C/A]GCATCTAAGGTCCTATTGTTACAAACT | | |
| | | AATGGGAGTTAGGAGTGATAAAGCTATATTGATGCAACTTTTTGGC | | |
| | | AGAAGATTGAGCGGAGAAGCTTTGGAACTGTTTAAATCTCACGAG | | |
| | | ACCAGACAATGAGATTGCTGGAATGCCTCGGCTAATGAATTCATT | | |
| | | GAGCGAGTTGCTTACAACGTAGAGATCGTCCCTGACT | | |
| 57 | TO-0201231 | GAACTATCTAACTTCTTTCATCCTCCTTTTATGCGATAGAGTTCATC | A | C |
| | SL2.50 | TTTATAAAAGTTTCTTTCTAACTCGTGCTTGCACGTATCTTTTAGAT | | |
| | 50267059 | CATGCCTCCATGAAGATTTGTCTGAGCTAGTCGTTCTAGGAGCAA | | |
| | | CGTTAAGGATCAAGAGGTGCCCAATGCACTAGAAGTGCGACCCTA | | |
| | | AGGAGAGGTCACTTCT[A/C]GGATCGGATAATGAGACAGATTGCG | | |
| | | ACCAACAAAGCTATGAAATAAAGAAAGAATTGACAGGAGCTAGTT | | |
| | | GATACTTCTAGCATTCGTGAGTACTTAAGGATGAATTTTTCAAGCT | | |
| | | TCAATAATTCAAGTGTGACTGAGGATCCAGAATATTTTATAAAATA | | |
| | | GTTGTAGAAAGTGATTGAGGTCATGCGTGTTGCTTATG | | |
| 58 | TO-0201232 | AGTTAGTGAGTTCTATAGTCTTTGGCAAGAGTTTTGGAGTATAATA | A | G |
| | SL2.50 | TGATGGTAAAAGCTTGTAAAAATGGTTTCCGTACTTACAATAGGAG | | |
| | 50344733 | TTTAAGGGGTCCGGGTACGACTCCCCAAATACCACCAAGGGGTC | | |
| | | CTTGAGGAGGACCCTAACACTGACCAAAGAGATTTCAAAACAACT | | |
| | | TGGTTGATGGTTGCAATTC[A/G]TGACCAGTAGGTTGGTCTACACC | | |
| | | CCGTAAGTCAAGTCATGCACCACTCCTACAAAGCTGATCCTCGGG | | |
| | | TGCAAACCACAAAGGTGGTTGACGTACAGTAACTTCTCCTACAGT | | |
| | | CCGTAGGTGGAGGGGTGTAGGTGTTTCCAGTTTTAAGTAATTTAT | | |
| | | TTTAGTTAGGTGTTTGGTAATCGATTAGTTATACAATTACGG | | |
| 59 | TO-0201233 | CATTAAGAGACTAAGGTCGTGGGGTAACAATCCATGGACCAGGAC | A | G |
| | SL2.50 | CACGACCTGTGACCTGGCCTAAGATCCGTGGATCTGGCAGTGGG | | |
| | 53891574 | TCGACCCTTAGAGGATTTTCTGGACCTTTTTGGGAAGGGTTGTAG | | |
| | | GTTGAAATCCACGGACCACCACTTACAGTCTGTGCGTCACCCTAG | | |
| | | TGGTAACTATGGATTGCACAT[G/A]TAGCTTCTGGAATTAACATGC | | |
| | | AACTTTTGGATGTTGGTTGGTCTTTTTAGGATAAAAGGTGTTACAT | | |
| | | TATCTCCCCCTTGGAAATAATCTTCCTCGAGTGAAGACTAAACTAG | | |
| | | CTGAATATGAAGGAAAAGAGCTTCCAACCCTACCACTAATTACTAG | | |
| | | AAACTGATTTCTGACTGAAATAAGTTCCAAGGACATGCAAA | | |
| 60 | TO-0202544 | TGAACATGTATTATTATAAGAATCCAATTAAAAACAAAATGAAACAC | G | A |
| | SL2.50 | TTGAAAGATCCGTACTTCCCAAAAAAAATTCCTCCTTCTCATGGCC | | |
| | | TTACAACAAAATTTATAAATTCTCTTTGTTAGAATAGAAATATGTAT | | |
| | 9317565 | ATATATATTCCTACTTAGAATAGGAAGAAGAATAGGAATAGGAATC | | |
| | | CTAGTTAGAAAAG[G/A]ACTTCAATGTATTGTCTATAAATAGGGTCT | | |
| | | CAATGTAACAATTTAGATACACAATTCAATAATATTTTTCCAATATA | | |
| | | TATTCTCACATGGTATCAGAGCA | | |

### SEED DEPOSITS

A sample of the S. lycopersicum seeds LVSTBRFVRES3 has been deposited by the Applicant, Vilmorin & Cie, 4 quai de la Megisserie, 75001 PARIS, France, pursuant to and in satisfaction of the requirements of the Budapest treaty on the International Recognition of the deposit of Microorganisms for the Purpose of Patent procedure (“the Budapest Treaty”) with the National collection of Industrial, Food and Marine bacteria (NCIMB) (NCIMB, Ltd, Ferguson Building, Craibstone Estate, Bucksburn, Aberdeen AB21 9YA, now Wellheads Place Dyce Aberdeen AB21 7 GB, United Kingdom) on 20 Feb. 2023 under accession number 44125. A deposit of this tomato seed is maintained by Vilmorin & Cie, 4 quai de la Mégisserie, 75001 PARIS, France.

A sample of the S. lycopersicum seed HAZTBRFVRES1 has been deposited by Hazera Seeds Ltd. Berurim, M. P. Shikmim 79837, Israel, pursuant to and in satisfaction of the requirements of the Budapest treaty on the International Recognition of the deposit of Microorganisms for the Purpose of Patent procedure (“the Budapest Treaty” with the National collection of Industrial, Food and Marine bacteria (NCIMB) (NCIMB, Ltd, Ferguson Building, Craibstone Estate, Bucksburn, Aberdeen AB21 9YA, now Wellheads Place Dyce Aberdeen AB21 7 GB, United Kingdom) on 16 May 2017 under accession number 42758. A deposit of this tomato seed is maintained by Hazera Seeds Ltd. Berurim, M. P. Shikmim 79837, Israel.

A sample of the S. lycopersicum seeds LVSTBRFVRES2 has been deposited by HM.Clause S.A., rue Louis Saillant, 26800 Portes-les-Valence, France, pursuant to and in satisfaction of the requirements of the Budapest treaty on the International Recognition of the deposit of Microorganisms for the Purpose of Patent procedure (“the Budapest Treaty”) with the National collection of Industrial, Food and Marine bacteria (NCIMB) (NCIMB, Ltd, Ferguson Building, Craibstone Estate, Bucksburn, Aberdeen AB21 9YA, now Wellheads Place Dyce Aberdeen AB21 7 GB, United Kingdom) on 1 Apr. 2020 under accession number 43591. A deposit of this tomato seed is maintained by HM.Clause S.A., rue Louis Saillant, 26800 Portes-les-Valence, France.

### LEGEND OF THE FIGURES

FIG. 1: this figure illustrates the results of a ToBRFV test carried out on different genotypes. The y-axis reports the scoring of the resistance, based on leaf symptoms, depending on the genotype (x-axis). The upper figure illustrates the results at 21 DPI (Scoring 2), and the lower at 14 DPI (Scoring 1).

FIG. 2: this figure illustrates the −log₁₀(Pᵥₐₗᵤₑ) along the different chromosomes; for an evaluation of the symptoms at 21 DPI.

FIG. 3: this figure is an illustration of the box plot of Tm-1 gene (referred to as QTL2 on this figure) and QTL8 alleles combination effect on ToBRFV leaf resistance after 21 and 28 DPI.

FIG. 4: this figure illustrates the −log₁₀(Pᵥₐₗᵤₑ) along the different chromosomes; for an evaluation of ToBRFV infection by qPCR.

FIG. 5A: this figure illustrates the qPCR data obtained for different genotypes with respect to TM-1 gene, QTL8 and QTL11 (corresponding to QTL3 according to WO2018/219941).

FIG. 5B: this figure illustrates the qPCR data obtained for different genotypes with respect to QTL8 and QTL11 (corresponding to QTL3 according to WO2018/219941).

FIG. 6: this figure illustrates the results of the ELISA tests, carried out at 1 month, 2 months and 5 months post inoculation, for different plants, namely the average of different susceptible controls, the mean of different plants of Source_K, Inbred IR, a plant known to delay the symptoms, however without finally restricting the viral replication, and for a non-inoculated control.

FIG. 7A: this figure illustrates the foliar symptoms on plants, mechanically inoculated by ToBRFV, depending on the presence of the QTL8 (homozygous (R), heterozygous (H), or absent(S)). The three different lines represent three different situations regarding the presence of a resistance QTL on chromosome 11 (either “QTL3” or “QTL11”, both represented as QTL11), (homozygous (R), heterozygous (H), or absent(S)).

FIG. 7B: this figure illustrates the foliar symptoms on plants, mechanically inoculated by ToBRFV, depending on the presence of the QTL8 (homozygous (R), heterozygous (H), or absent(S)); and depending also on the presence of Tm-1 (homozygous (R), heterozygous (H), or absent(S)).

FIG. 8A. this figure illustrates the results of qPCR (viral multiplication), for different genotypes. Elite corresponds to plants comprising QTL3, (RSRR) corresponds to the genotype of plants comprising QTL8 heterozygously and QTL11 homozygously, (RSRS) corresponds to the genotype of plants comprising QTL8 heterozygously and QTL11 heterozygously, (SSRR) corresponds to the genotype of plants comprising QTL11 homozygously and not comprising QTL8 and (SSRS) corresponds to the genotype of plants comprising QTL11 heterozygously and not comprising QTL8.

HR (highly resistant) means that there is no viral multiplication. R (resistant) means that there is viral multiplication but at very low level; RI (intermediate resistant) means viral multiplication at low level; and S (susceptible) represents viral replication.

FIG. 8B.: this figure illustrates the results of foliar symptoms scoring at 28 post inoculation, for the same different phenotypes as FIG. 8A. A score of 1 or 2 is indicative of susceptible plants/genotypes in these conditions, a score of 4 or 5 is indicative of plants/genotypes with intermediate resistance and a score of 7 to 9 is indicative of resistant plants/genotypes, without symptoms of infection.

### EXPERIMENTAL SECTION

### Example 1: Identification of a Wild Solanum Habrochaites Accession as a Resistance Source Against ToBRFV

### 1.1 First Trial—Laboratory Screening in Israel

The inventors screened a wide range of more than 800 accessions of the various wild relatives of cultivated tomato. A wild accession from Solanum habrochaites was identified and further tested with a susceptible control for comparison, applying the protocols disclosed below.

Inoculation: the seedlings are mechanically inoculated with an inoculum of an isolate of ToBRFV ToBRFV-H1i1 (Israeli isolate), at the stage of second true leave.

The inoculum is applied on two young leaves of the plants to be tested, by rubbing gently these leaves with a rough sponge soaked in the inoculum.

### Immunostrip

Presence of ToBRFV in the plants is checked by using a TMV immunostrip (this immunostrip is not specific to TMV and recognizes also ToBRFV).

### ELISA

ELISA were performed using the Serum kit TMV DAS-ELISA from Agdia (ref. SRA57400), following the protocol recommended by the manufacturer.

### Trial:

The plants to be tested were sown on 15.07 and inoculated 2 weeks later, on 29.07. They were then scored (phenotypically+“immunostrip” assay) one month after inoculation, on 30/08. The sampling for ELISA was carried out on 10.09.

The results of the ToBRFV phenotypic assays and of the immunostrip assay are reported in table 1, for the identified wild Solanum habrochaites accession and for line 997, which is a susceptible control.

TABLE 1

| | | | | ToBRFV |
|---|---|---|---|---|
| | | Healthy, | Symptomless, | symptoms, |
| | Total no. | negative | but positive | positive |
| Accession/ | of | immunostrip | immunostrip | immunostrip |
| line | plants | test | test | test |
| Wild S. | 34 | 32 | 0 | 2 |
| habrochaites | | | | |
| accession of | | | | |
| interest | | | | |
| 997 (suscept. | 60 | 0 | 0 | 60 |
| Control) | | | | |

The results of the ToBRFV ELISA test are reported in table 2. More specifically, 27 plants of the accession of interest which were scored as “healthy and negative immunostrip test” in table 1 one month after inoculation, were then tested by ELISA around 10 days later.

TABLE 2

| | | | Results which cannot be | |
|---|---|---|---|---|
| | Total no. of | Negative | attributed clearly to positive | Positive |
| Accession/line | plants | ELISA result | or negative result | ELISA result |
| Wild S. habrochaites | 27 | 4 | 12 | 11 |
| accession of interest | | | | |
| 997 - susceptible control | 6 | 0 | 0 | 6 |

Conclusion of the trial: the S. habrochaites accession of interest seems to be segregating for ToBRFV resistance. Some of the plants of this accession are however highly resistant to ToBRFV and could constitute a source of resistance for cultivated tomatoes.

### 1.2 Second Trial—Laboratory Screening

Plants of the tested wild accession's genotype and F1 derived from the cross between this accession and a S. lycopersicum genotype were tested for resistance to ToBRFV. Two susceptible genotypes, Momor and Moneyberg, were used as control.

Two ToBRFV isolates were used to perform the Bioassay: isolate ToBRFV-H2i1 and isolate ToBRFV-H3i1, corresponding to a Jordan isolate (Jordan_2015) and a South European isolate (2018) respectively. It is indeed known that different strains of ToBRFV are circulating, with different levels of aggressivity; especially the strains from ToBRFV-H3 pathotype are known to be more aggressive than the ToBRFV-H1 and ToBRFV-H2 pathotypes.

Virus isolates are maintained by frozen storage of infectious leaves coming from 14 days old, infected tomato. Bioassays are carried out by sap-inoculation of tomato plantlets at two-leaf stage (i.e. 14-16 days after seeding) by rubbing the cotyledons with the index finger. Plants were kept in a S3 phytotron, with 20° C. night and 24° C. day, with a photoperiod of 13 hours.

Systemic symptoms evaluations are carried out by plant-by-plant scoring at 14 and 21 days post-inoculation (DPI), a last evaluation at 28 DPI is optionally carried out on some plants.

Symptoms are visually assessed according to the following scale:
9: No visible symptoms
7: small phenotypic differences, but not clear if due to the virus or not
5: moderate symptoms (mosaic and/or light vein banding)
3: strong/severe symptoms (strong mosaic and/or pronounced vein banding and/or small leaves deformation)
1: very strong/severe symptoms (leaves deformation and/or mosaic and/or highly pronounced vein banding).

Plants having notes 9 and 7 are considered Resistant (R), while plants scored 5, 3 or 1 are considered Susceptible(S).

The sampling for the qPCR is carried out at 28 DPI, i.e. around 1 month after inoculation.

The phenotyping results are detailed in table 3. In this table R and S refer to ToBRFV Resistant and Susceptible phenotype respectively.

TABLE 3

| | ToBRFV-H2i1 | | ToBRFV-H3i1 | |
|---|---|---|---|---|
| | isolate | | isolate | |
| Genotype | R | S | R | S |
| Wild S. habrochaites accession | 78 | 1 | 87 | 0 |
| of interest | | | | |
| F1 | 5 | 2 | 9 | 2 |
| Susceptible Momor | 0 | 9 | 0 | 8 |
| Susceptible Moneyberg | 0 | 9 | 0 | 9 |

After 28 days of test, the plants without symptoms are tested by quantitative PCR with Taqman probe to evaluate the presence of ToBRFV in plants compared to non-inoculated plants. The protocol for the molecular quantification of ToBRFV by qPCR is as follows:

The young wrapper leaf at head grown are sampled (3 to 4 leaves per plant). The leaves are ground in liquid nitrogen and an aliquot of 100 mg is kept for RNA extraction. For each sample, 100 mg of grounded leaves are used for RNA extraction

RNA extraction is performed using the «Maxwell® 16 LEV Plant RNA Kit» from Promega® and the Maxwell® extraction robot (Promega®). Extracted RNA are stored at −20° C. qPCR for virus quantification is performed using the TaqMan universal Master Mix (ThermoFisher Scientific®) with Kit Gotaq Probe OneStep RTqPCR system A6120 Promega, following the manufacturer instructions.

Primers and probes used are disclosed in table 4 below:

| primer | Sequence | SEQ ID NO |
|---|---|---|
| LM_TBRFV-1-F | AGATTTCCCTGGCTTTTGGA | SEQ ID NO. 26 |
| LM_TBRFV-1-R | CTCTTTCTGATATCAAGCACT | SEQ ID NO. 27 |
| LM_TBRFV-1-probe | CAAGGAGAGACTGCTAAATCGG | SEQ ID NO. 28 |

The expected amplified fragment is 187 bp in case of presence of the viral genome and hybridization with the probe.

### Mix:

The Kit TaqMan universal Master Mix, Applied Biosystem ThermoFisher Scientific is used.

The total volume of the mix is 20 μL (comprising 1 μL of the reverse and forward primers at 0.5 μM and 0.5 μM of probe at 0.25 μM); 2 μL of RNA are added; such that the final volume is 22 μL.
Thermocycler: Reverse transcription: 15 minutes at 45° C._Inactivation of Reverse transcription and
Activation: 2 minutes at 95° C.;
40 cycles comprising: Denaturation: 15 seconds at 95° C.,
   Annealing primers: 15 seconds at 54° C.; and
   Annealing probes: 30 seconds at 48° C.

For each sample, 3 replicates are performed. Standard dilution curves are used for the relative quantification. A melting curve is performed at the end of the protocol in the StepOne® to ensure the specificity of the detection/quantification.

The fluorescence of the probe is measured in real time, for each sample. The evolution of the fluorescence is followed as a function of the number of amplification cycles of qPCR, and a number of cycles, named Ct or Cq is defined for each sample.

Ct means “Crossing threshold” or “crossing point”, it is also named Cq or Cp, and it is the number of amplification cycles beyond which the amplification signal is considered as significative, i.e. above the margin of error or noise.

The Ct obtained for non-inoculated controls (negative template) corresponds to the threshold beyond which the virus is no longer detected. Any value of amplification cycles above this Ct therefore only means absence of the viral sequences.

The Ct of each sample is reported on the standard curve to calculate the relative quantity of virus in each sample. The sample is considered to be negative, i.e. to not replicate the virus after inoculation, when the measured Ct is above the Ct measured for non-inoculated plants.

The results obtained are reported in table 5:

TABLE 5

| | ToBRFV-H2i1 | | ToBRFV-H3i1 | |
|---|---|---|---|---|
| | isolate | | isolate | |
| Genotype | positive | negative | Positive | negative |
| Wild S. habrochaites accession | 1 | 5 | 0 | 6 |
| of interest | | | | |
| F1 | 2 | 2 | 0 | 4 |

The seed lot of the S. habrochaites accession of interest tested in this experiment, displaying resistance to ToBRFV is segregating for ToBRFV resistance. F1 lots were generated from this source, and one lot, fixed for the resistance, was called Source_K.

### Example 2: Development of BC1 Population

A backcross population has been developed by crossing resistant source_K plant with a susceptible elite parent, called “Elite 1” in the following, using Elite 1 as recurrent parent. Elite 1 is a breeding line, having indeterminate round fruits. This line contains the Tm2.2 resistance gene and QTL3 (i.e. QTL on chromosome 11 providing tolerance to ToBRFV, described as “QTL3” in patent application WO2018/219941) both homozygously. Elite 1 as well as Source_K do not comprise the tolerance QTL9, on chromosome 9, disclosed in application WO2018/219941 (called “QTL2” in that prior document), as checked by using the SNPs markers disclosed in WO2018/219941.

In the following table, “RR” means that the resistance gene (Tm-1 or Tm2.2) or QTL (QTL9 or QTL3) is present homozygously as checked by representative markers. “SS” means that the resistance genes are absent, or that a susceptible allele of the gene/QTL is present homozygously.

“NT” means that the phenotype and/or genotype have not been tested.

| | TM1 | TM2.2 | QTL9 | QTL3 |
|---|---|---|---|---|
| Elite 1 | SS | RR | SS | RR |
| Source K | RR | NT | SS | SS |

### Example 3: QTL Mapping in Source_K for ToBRFV Leaf Resistance on Leaf Symptoms and on Virus Titer

130 plants from the BC1 population, 8 plants of both parents Source K and Elite 1 and 6 plants of the F1, were screened for the resistance to ToBRFV with the protocol described in section 1.2 using the ToBRFV-H3i1 isolate. All plants were sampled for relative virus quantification using qPCR.

One month after sowing, a leaf sampling is carried out, DNA is extracted for genotyping the plants.

The inoculation is made around one month after sowing. The first and second phenotypic scorings are made at 14 and 21 DPI respectively, a last evaluation at 28 DPI is optionally carried out; simultaneously, the leaves are sampled for qPCR.

### Results:

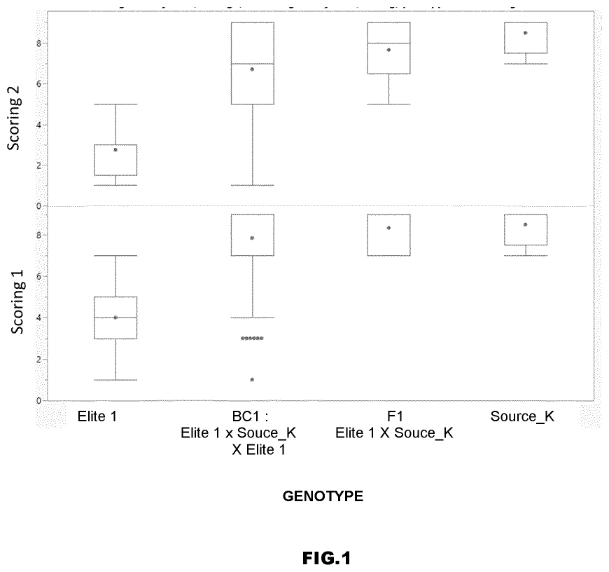

After the 2 scorings, a segregation was observed in the BC1 population as shown on FIG. 1.

Quantitative PCR was performed for each plant samples, the parents and non-inoculated plants that were not grown in the same phytotron to avoid contact contamination (negative control) with (i) ToBRFV taqman protocol described in part 1.2 and (ii) the housekeeping gene Ubiquitine (UBI) protocol.

The commercial kit used is the Kit Gotaq OneStep RTqPCR; it is used in combination with the system A6020 Promega.

The qPCR cycle lasts 1 h 15 and is as follows:
Reverse transcription: 15 minutes at 45° C.
Inactivation of the reverse transcription and activation of Go Taq: 2 minutes at 95° C.
And then 40 cycles: denaturation: 15 seconds at 95° C. and Annealing/elongation: 30 seconds at 60° C.
For the Ubiquitin, the primers are those disclosed in Mascia et al 2010 and are as follows:
UBI forward primer: TCGTAAGGAGTGCCCTAATGCTGA (SEQ ID NO.29) Tm=62.7° C.
UBI reverse primer: CAATCGCCTCCAGCCTTGTTGTAA (SEQ ID NO.30) Tm=62.7° C.

The relative quantification of virus in each plant was then calculated using the Pfaffl method (Pfaffl, 2001). Plants with a relative quantity of virus which is inferior to the quantity of virus in the non-inoculated plants, as measured by the cycle numbers, are considered as negative (immune).

130 individuals of a BC1 population ((Elite 1*Source_K)*Elite 1) were genotyped with set of 782 SNPs (midplex) and Tm1, Tm2.2 and S. habrochaites specific markers.

### Results of the Mapping Using ToBRFV Leaf Symptom Score.

The QTL analysis was done using ANOVA model for biparental population in a software module platform proprietary of the applicant, using ToBRFV leaf symptom score (applying the symptom scale described in example 1.2) at 14 and 21 DPI.

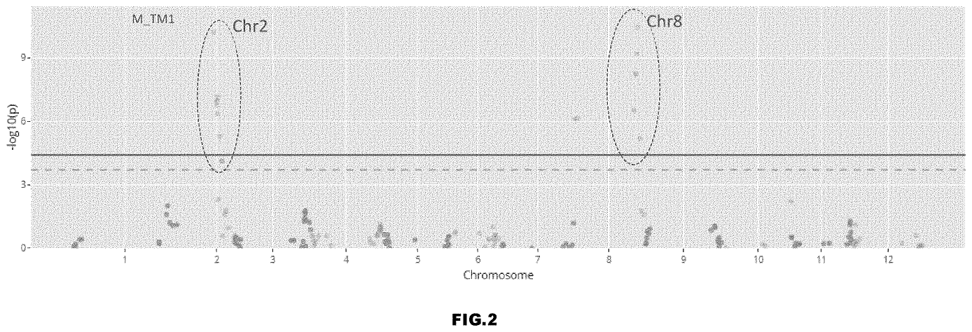

The results presented in FIG. 2 and table 7 show that 2 major QTL were detected in the BC1 source_K*Elite 1 population, on chromosome 2 and 8, on the basis of the ToBRFV leaf symptom score. The markers significantly linked to ToBRFV leave resistance after 21 DPI are described in Table 6. The best linked marker on chromosome 2 (TO-0178073) is linked to Tm-1 gene.

FIG. 2 illustrates the −log₁₀(Pᵥₐₗᵤₑ) by chromosome for ToBRFV leaf symptom score at 21 days post inoculation

TABLE 6
Markers linked to QTL on chromosome 2 and 8, as evaluated by
ToBRFV leaf symptom score at 21 DPI.

| | Chromo- | | Allele | Allele |
|---|---|---|---|---|
| Marker | some | Pvalue.Locus | Elite 1 | Source K |
| TO-0178073 | 2 | 6.16E−11 | G/G | A/A |
| SEQ ID NO: 31 | | | | |
| TO-0202572 | 8 | 3.79E−11 | C/C | A/A |
| SEQ ID NO: 32 | | | | |

TABLE 7
Results of the QTL detection for the trait corresponding to resistance at 21 DPI by ANOVA. For
each SNP, an homozygous allele was arbitrarily chosen (allele 1) as well as its effect on resistance
(Effect 1) as reference. Presence of the marker at the heterozygous stage (Allele htz) and the
associated level of resistance is then assessed (Effect htz). The level of significance of the
association between the trait and the locus is indicated by the pvalue and LOD score. “R2 locus”
represent an estimate of the proportion of trait variability associated with the locus.

| | | Pos | Allele | Effect | Allele | Effect | Pvalue. | | R2. |
|---|---|---|---|---|---|---|---|---|---|
| Locus | CHR | SL2.50 | 1 | 1 | htz | htz | Locus | LOD | Locus |
| TO-0178073 | 2 | 34 285 077 | G/G | −1.33 | A/G | 1.33 | 6.16E−11 | 10.21 | 0.30 |
| TO-0162501 | 2 | 37 321 323 | A/A | −1.11 | A/G | 1.11 | 1.03E−07 | 6.99 | 0.20 |
| TO-0162502 | 2 | 37 328 686 | A/A | −1.11 | A/T | 1.11 | 1.03E−07 | 6.99 | 0.20 |
| TO-0162503 | 2 | 37 342 653 | G/G | −1.09 | A/G | 1.09 | 1.73E−07 | 6.76 | 0.20 |
| TO-0121094 | 2 | 37 647 707 | G/G | −1.12 | A/G | 1.12 | 6.67E−08 | 7.18 | 0.21 |
| TO-0080945 | 2 | 38 040 631 | A/A | −1.05 | A/G | 1.05 | 4.37E−07 | 6.36 | 0.18 |
| TO-0100566 | 2 | 40 037 728 | A/A | −0.95 | A/G | 0.95 | 5.13E−06 | 5.29 | 0.15 |
| TO-0100946 | 2 | 41 845 519 | G/G | −0.84 | A/G | 0.84 | 7.79E−05 | 4.11 | 0.12 |
| TO-0121826 | 2 | 42 019 898 | G/G | −0.84 | A/G | 0.84 | 7.79E−05 | 4.11 | 0.12 |
| TO-0109334 | 8 | 1 198 982 | A/A | −1.07 | A/C | 1.07 | 7.95E−07 | 6.10 | 0.18 |
| TO-0143197 | 8 | 3 498 192 | A/A | −1.09 | A/G | 1.09 | 7.51E−07 | 6.12 | 0.18 |
| TO-0142175 | 8 | 55 964 965 | G/G | −1.12 | A/G | 1.12 | 3.10E−07 | 6.51 | 0.19 |
| TO-0121802 | 8 | 57 576 808 | G/G | −1.24 | A/G | 1.24 | 5.85E−09 | 8.23 | 0.23 |
| TO-0121799 | 8 | 57 915 637 | A/A | −1.24 | A/G | 1.24 | 5.81E−09 | 8.24 | 0.24 |
| TO-0102000 | 8 | 58 963 339 | A/A | −1.28 | A/C | 1.28 | 6.60E−10 | 9.18 | 0.26 |
| TO-0202572 | 8 | 59 780 025 | C/C | −1.35 | A/C | 1.35 | 3.79E−11 | 10.42 | 0.30 |
| TO-0101270 | 8 | 61 842 894 | A/A | −0.96 | A/G | 0.96 | 6.64E−06 | 5.18 | 0.15 |

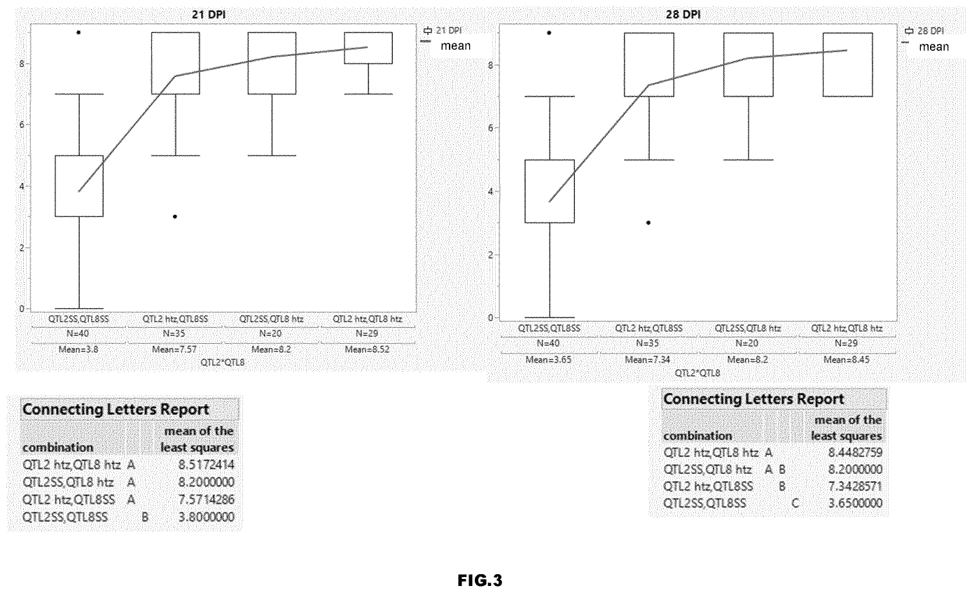

The ANOVA performed to evaluate the effect of Tm-1 and QTL8 combination on ToBRFV leaves resistance at 21 and 28 DPI shows significant effect of QTL8 and Tm-1, individually and in combination. (FIG. 3, box plot).

### Results of the Mapping Using Virus Titer.

The mapping is then conducted on the basis of virus titer measured by qPCR as detailed above.

The QTL analysis was performed using ANOVA model for biparental population in a software modules platform proprietary of the Applicant) using ToBRFV Ct score.

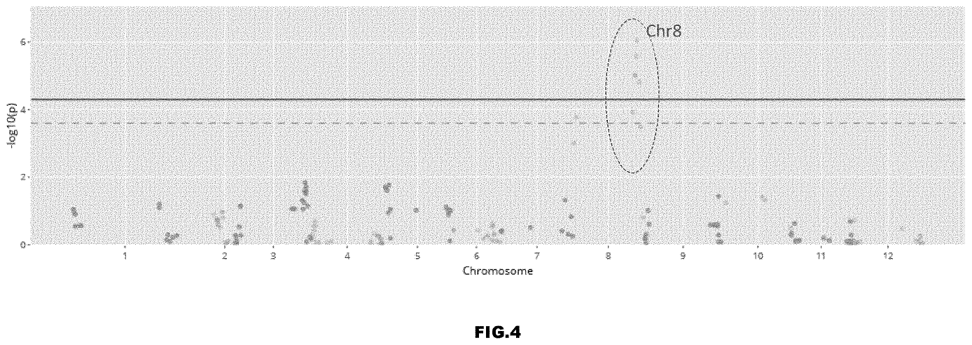

The results presented in FIG. 4 and table 8 show that QTL on chromosome 8 detected for ToBRFV leaf resistance is also significantly associated to ToBRFV Ct (virus presence in leaf). The marker significantly linked to ToBRFV Ct is TO-0202572.

TABLE 8
ANOVA results for ToBRFV Ct
Variable: Ct ToBRFV

| | | Position | Allele | | Allele | Effect | Pvalue. | | R2. |
|---|---|---|---|---|---|---|---|---|---|
| Locus | CHR | SL2.50 | 1 | Effect1 | htz | htz | Locus | LOD | Locus |
| TO-010933 | 8 | 1 198 982 | A/A | −2.75 | A/C | 2.75 | 2.75E−07 | 6.56 | 0.13 |
| TO-0143197 | 8 | 3 498 192 | A/A | −3.31 | A/G | 3.31 | 5.46E−10 | 9.26 | 0.12 |
| TO-0142175 | 8 | 55 964 965 | G/G | −3.29 | A/G | 3.29 | 5.80E−10 | 9.24 | 0.11 |
| TO-0121802 | 8 | 57 576 808 | G/G | −3.69 | A/G | 3.69 | 6.91E−13 | 12.16 | 0.15 |
| TO-0121799 | 8 | 57 915 637 | A/A | −3.69 | A/G | 3.69 | 6.91E−13 | 12.16 | 0.15 |
| TO-0102000 | 8 | 58 963 339 | A/A | −3.90 | A/C | 3.90 | 3.63E−15 | 14.44 | 0.17 |
| TO-0202572 | 8 | 59 780 025 | C/C | −4.31 | A/C | 4.31 | 3.83E−20 | 19.42 | 0.19 |
| TO-0101270 | 8 | 61 842 894 | A/A | −3.45 | A/G | 3.45 | 3.17E−12 | 11.50 | 0.12 |
| TO-0101274 | 8 | 62 865 754 | C/C | −2.43 | A/C | 2.43 | 8.91E−06 | 5.05 | 0.06 |

### Combination of Tm-1, QTL8 and QTL3:

The qPCR data obtained for the parents (Source_K, Elite 1), for two different F1 and for the BC1 described in example 2, were analyzed depending on the genotype of the plants with respect to Tm-1 (Tm-1 gene), QTL8 and QTL3. The 8 different genomic combinations are (RRR), (RRS), (RSR), (RSS), (SRR), (SRS), (SSR), (SSS), wherein R stands for Resistance and S for Susceptible, and the letter appearing first represents Tm-1 gene, the second letter QTL8 and the third letter QTL3. Tm-1 resistance gene and QTL8 having an expected dominant effect, the plants are noted R if the Tm-1 gene or QTL8 is present, either homozygously or heterozygously. For QTL3, the plant is noted “R” only if QTL3 is present homozygously and S if the plant is heterozygous for QTL3, given that QTL3 is known to be recessive.

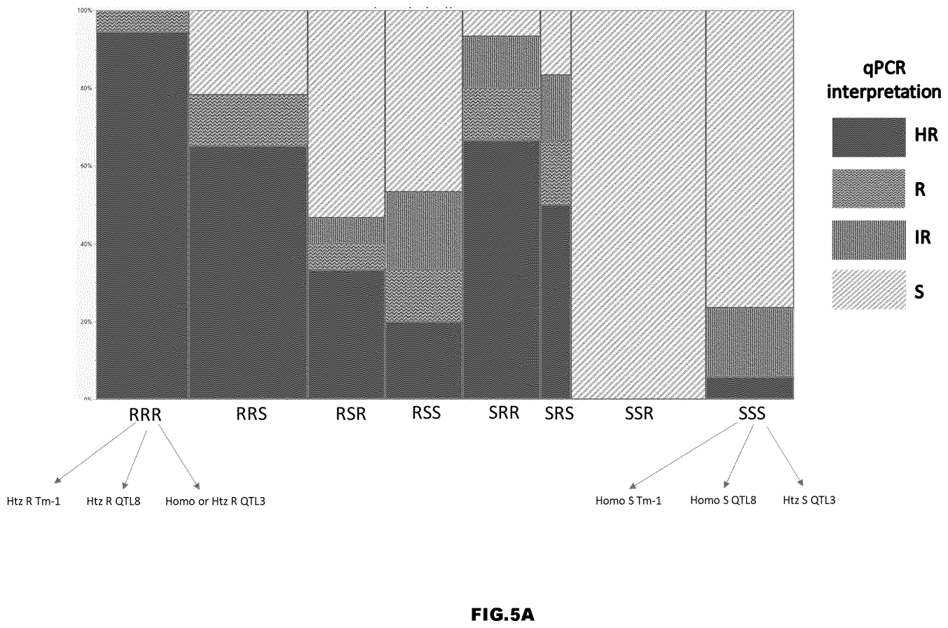

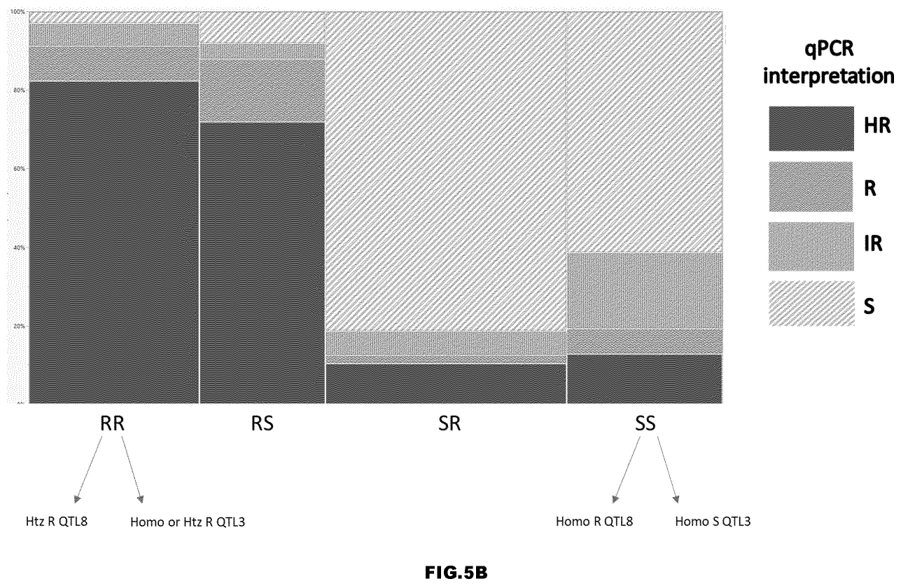

The results for these 8 different genotypes, as obtained from the BC1 population and parent screening, are illustrated in FIG. 5A. On this figure, the upper region (diagonal hatched) relates to plants in which the viral concentration is important; these plants are considered as susceptible to ToBRFV(S). The region below (vertical hatched) corresponds to plants considered as intermediate resistant (IR), i.e. plants in which the viral concentration as measured by qPCR is between 1000 times less and 100 times less than within the positive control Elite 1 (susceptible). The region below (wavy) corresponds to plants considered as resistant (R), i.e. plants in which the viral concentration as measured by qPCR is at most 1000 times less than within the positive control Elite 1 (susceptible). The bottom region (gray area) corresponds to plants considered as highly resistant (HR), i.e. plants in which the viral concentration as measured by qPCR is less than within non-inoculated control Elite 1 (susceptible), i.e. below the detection level.

It can be inferred from these results that the plants exhibiting the lowest level of viral multiplication (and thus the highest number of cycles in qPCR) are those bearing the QTL8; this feature is improved by the combination with Tm-1 resistance gene and also by QTL3. The combination of these 3 genetic determinants gives an even lower level of viral multiplication and thus increased resistance.

The same analysis has been carried out with the combination of QTL8 and QTL3. The four haplotypes are (RR), (RS), (SR) and (SS), wherein the 1^{st} letter refers to QTL8 presence (which is dominant) and the 2nd letter to the QTL3, normally recessive for the ToBRFV tolerance. “R” is thus attributed to a plant with respect to QTL3 only when this QTL is present homozygously.

The results for these 4 different haplotypes, as obtained from the BC1 population and parent screening are illustrated in FIG. 5B. It can be inferred from these results that the plants exhibiting the lowest level of viral multiplication (and thus the highest number of cycles in qPCR) are those bearing the QTL8 as obtained by the inventors, in combination with QTL3.

### Example 4: QTL8 Region Analysis

The interval on tomato genome assembly SL3.0 ranges from 59100098 to 61/782,030 and contains 292 genes. The Solanum lycopersicum knowledge graph (v51) from Knetminer was used to investigate functions represented by these genes. A total of 45 traits were represented within the interval. Forty-one genes (out of 2566 in the genome) were annotated with a “Disease resistance” trait showing the possible existence of a resistance gene cluster. Among them, 5 genes (out of 219 in the genome) were annotated with the Gene Ontology Biological Process term “Defense response to virus” (Solyc08g075980.1, Solyc08g075825.1 (RDR6), Solyc08g076000.3, Solyc08g075640.3 and Solyc08g075630.3).

The QTL region in S. lycopersicum was first compared to a S. habrochaites genome. A genome assembly was very recently released for the S. habrochaites LA1353 (Seong et al 2022). Using a whole-genome pairwise aligner, chromosome 8 of S. lycopersicum (according to the genome assembly SL4.0) was found to match with scaffold64 of LA1353.

Although the region was globally well conserved between SL4.0 and LA1353 (82%), some important variations could be observed, with some sequences in LA1353 which were not mapped against SL4.0 revealing the presence of some additional genes in the S. habrochaites accession. Using a blastp search, 541 possible gene models were detected in this region in LA1353.

The inventors then generated a whole genome resequencing of Source_K (on the basis of the resistant plants, as this source segregates for the resistance) using Illumina short reads (Eurofins). A total of 86,589,017 cleaned reads (trimmed with Fastp v0.20) were obtained and 93% read pairs were properly mapped against LA1353 genome assembly (vs 80% when using SL4.0 as a reference genome). Variants were called using the GATK tool suite v4.2 and following the recommended best practices. The inventors found 20,175,094 variations and use them to construct predicted gene sequences with the bcftools consensus tool v1.9 and considering possible heterozygous alleles. Using this approach, 246 coding and protein sequences could be predicted in Source_K.

A pairwise comparison of predicted protein sequences from LA1353 and Source_K indicated an overall high percentage of identity around the possible causal gene. This identity was lower when comparing to SL4.0 (min 38.2%, mean 91.5%, max 100%). These pairwise comparisons were able to detect at least 2 blocks of genes with high identity, probably corresponding to similar functional domains. This suggests the existence of NBS-LRR type gene clusters in this specific region.

Gene prediction in LA1353 was performed using short read RNA-seq from diverse S. habrochaites but without iso-seq supporting data. Some gene models in LA1353 could therefore be erroneous especially for duplicated genes harboring very similar sequence stretches.

A full-length high confidence sequence homologous to seq116 of WO2020148021 (SEQ ID NO:33) was detected in Source_K.

The consensus sequence of the gene, potentially present as multiple copies, identified in the resistant plants of Source_K, encodes a protein, homologous to SEQ ID No:116 of WO2020148021, and having the sequence:

(SEQ ID NO: 1)
MAEAFLQIMLENLTCFIQGELGLILGFKDEFEKLQSTFTTIQAVVQDAQLKQLKDKAIENWLQKLNGAAYEA
DDILDECKTEAPIIQKKNKYGCYHPNVITFRHKIGKRMKKIMEKLDAIAAERIKFHLDERTIERQVATRQTGF
VLNEPQVYGRDKDKDEIVKILINNAQTLSVLPILGMGGLGKTTLAQMVFNDQRVIEHFHPKIWICVSEDFNE
KRLIKEIVESIEEKSLGDMDLAPLQKKLQDLLNGKKYLLVLDDVWNEDQDKWAKLRQVLKAGASGAYVLT
TTRLEKVGSIMGALQPYELSNLSQEDCWLLFMQRAFGHQEEMNLNLVAIGKVIVKKCGGVPLAAKTLGGI
LRFKREERQWEHVRDNEIWSLPQDESSILPSLRLSYHHLPLDLRQCFAYCAVFPKDTKMIKENLITLWMA
HGFLLSKGNLELEDVGNEVWNELYLRCFFQEIEAKSGNTYFKIHDLIHDLATSLFSASTSSSNIREINVEGY
LHMMSIGFAKVVSSYSPPHLQKFVSLRVLNLSSMGLKQLPSSIGDLVHLRYLNLSLNNMRTLPKQLCKLQ
NLQTLNVEYCWSLCCLPKETSKLGSLRNLLLDGCDGLDSMPPRIGSLTCLKTLSFFVIGERKDSLLGELRN
LNLYGSIEITHLERVKNDRDAKEANLSAKENLHSLSMRWKGPHRYESEEVEVLESLKPHPNLTSLLITGFR
GFRLPKWMNHSVLKNVVSIAIRGCENCSCLPPFGDLPCLESLELGDGSAELEYVEDSGFPTRRRFPSLRK
LIIVNFDNLKGLLKEAGEEQFPVLEEMTISWCPVLVIPTLSSVKKLVVYRNMSDAIGLRSIYNLRALTSLNISH
NLTATSLPEEMFKSLANLKYLEISFIFNLKELPNSLASLNALKHLKIEYCDALESLPEEGVKGLTSLTELSITN
CKRLKCLPEGLQHLTNLSVRECPTLAKRCEKGIGQDWYKIAHIPHLL.

The differences in sequences confirm that Source_K and the resistance gene are different from the Source used in WO2020148021 and from the other sources suggested in Jewehan, 2022.

### Example 5: ToBRFV Resistance from Source_K at an Adult Plant Stage

In order to evaluate the level of ToBRFV resistance of Source_K at an adult plant stage, a greenhouse trial was carried out in Israel.

### Material & Methods

The trial was based on mechanical inoculation of seedlings with ToBRFV-H1i1 isolate of ToBRFV at the stage of second true leave.

The controls in this trial are as follows:
1. Susceptible controls—
   Haz. Tm-S—an indeterminate cultivated parental line which has no resistance to ToMV, known as expressing most severe ToBRFV foliar symptoms.
   Haz. Tm-R—an indeterminate cultivated parental line which has the Tm-22 resistance gene at homozygotic state, known as expressing moderate-severe ToBRFV foliar symptoms.
2. Resistant control—
   Inbred IR—an indeterminate cultivated parental line which has the resistance gene Tm-1 and QTL3 (a QTL on chromosome 11 as described in WO2018/219941), both at homozygous state, known as resistant to ToBRFV foliar and fruit symptoms. In this control line—when inoculated by ToBRFV—it is known to delay the virus accumulation at the early stage of growth (compared to susceptible genotypes).
3. Non-inoculated, healthy control—grown in a separate greenhouse.

The phenotypic evaluation was carried out on the basis of foliar symptoms and the scoring scale is as detailed in example 1.2. The Elisa test is as detailed in example 1.1.

Because the ELISA assay detects both infectious virions and non-infectious virus particles, the assay is followed by a bioassay to confirm the presence or absence of infectious form of ToBRFV. The bioassay is carried out on leaves of N. tabacum cv. xanthi; infectious virions cause typical local lesions that demonstrate viability of the virus.

Specifically, the entire surface of two fully expanded consecutive leaves of N. tabacum cv. Xanthi NN plants at the 4-5 true leaves stage are inoculated with a leaves extract of the tested tomato plants. The tobacco plants are then inoculated during at least 5 days before the lesions on the leaves are observed.

### Timetable.

Sowing;
Mechanical inoculation: 13 days later;
Transplanting in the quarantine: around one month after sowing;
1^{st} Sampling for ELISA: around 40 days after inoculation (i.e. around one month);
1^{st} scoring (foliar symptoms): 6 weeks after inoculation;
2^{nd} Sampling for ELISA: around 9 weeks after inoculation (i.e. around 2 months);
2^{nd} scoring (foliar symptoms): between 9 and 10 weeks after inoculation;
3^{rd} Sampling for ELISA: around 5 months after inoculation;
3^{rd} scoring (foliar symptoms): around 5 months after inoculation;
Sampling for bioassay on Tobacco cv. Xanthii: between 5 and 6 months after inoculation.

### Results

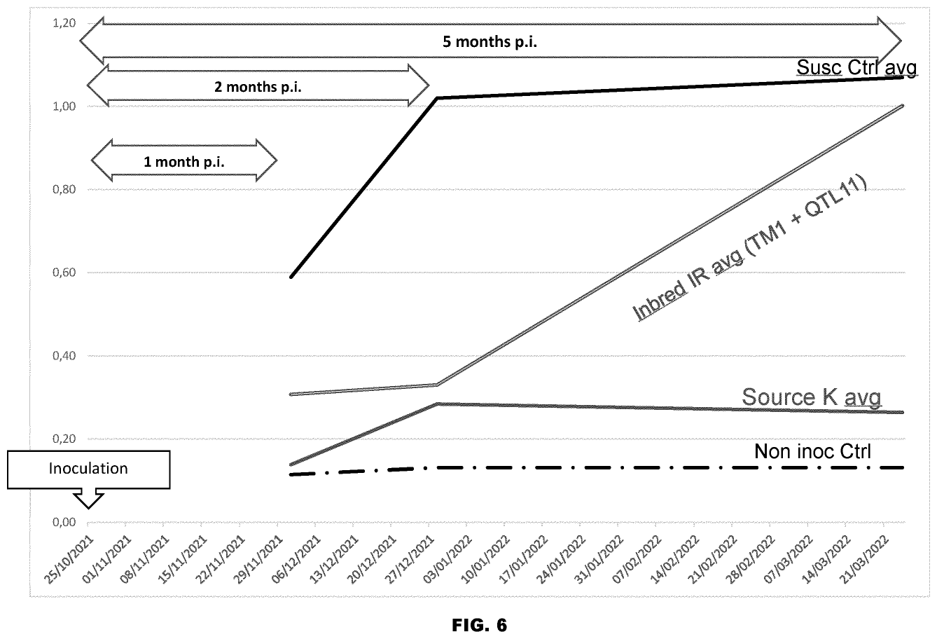

Table 9 reports the results of the 1^{st} scoring based on the phenotype (foliar symptoms) and ELISA assay, 5 to 6 weeks after inoculation. The results of the ELISA assay are also illustrated on FIG. 6 for the mean of Source_K, the mean of Inbred IR and the mean of healthy non-inoculated controls.

“Nb of +”; “Nb of −” and “%+” stand for “number of positives”, “number of negatives” and “percentage of positives” respectively.

TABLE 9

| | | Foliar Symptoms | | | | | | | Elisa | | |
|---|---|---|---|---|---|---|---|---|---|---|---|
| | Nb of | Score | | | | | | Std | Nb | Nb | |
| | plants | 1 | 3 | 5 | 7 | 9 | mean | Dev | of + | of − | % + |
| Source_K | 15 | 0 | 0 | 0 | 0 | 15 | 9 | 0 | 0 | 15 | 0 |
| Inbred IR | 20 | 0 | 0 | 0 | 0 | 20 | 9 | 0 | 5 | 15 | 25 |
| Haz Tm-S | 20 | 20 | 0 | 0 | 0 | 0 | 1 | 0 | 10 | 0 | 100 |
| Haz Tm-R | 14 | 6 | 8 | 0 | 0 | 0 | 2.1429 | 1.02711 | 14 | 0 | 100 |
| Healthy control | 8 | 0 | 0 | 0 | 0 | 8 | 9 | 0 | 0 | 8 | 0 |

Table 10 reports the results of the 2^{nd} scoring according to the phenotype (foliar symptoms) 9 to 10 weeks after inoculation. The results of the ELISA assay are illustrated on FIG. 6.

Plants of Source K have a very low ELISA results, level of virus concentration.

TABLE 10

| | | Foliar Symptoms | | | | | | |
|---|---|---|---|---|---|---|---|---|
| | Nb of | Score | | | | | | Std |
| | plants | 1 | 3 | 5 | 7 | 9 | mean | Dev |
| Source_K | 15 | 0 | 0 | 0 | 0 | 15 | 0 | 0 |
| Inbred IR | 20 | 0 | 0 | 0 | 0 | 20 | 9 | 0 |
| Haz Tm-S | 20 | 20 | 0 | 0 | 0 | 0 | 1 | 0 |
| Haz Tm-R | 14 | 6 | 8 | 0 | 0 | 0 | 2.1429 | 1.02711 |
| Healthy control | 8 | 0 | 0 | 0 | 0 | 8 | 9 | 0 |

Table 11 reports the results of the 3^{rd} scoring according to the phenotype (foliar symptoms), 150 days after inoculation (around 5 months).

“nt” stands for not tested.

The results of the ELISA assay are illustrated on FIG. 6.

TABLE 11

| | | Foliar Symptoms | | | | | | |
|---|---|---|---|---|---|---|---|---|
| | Nb of | Score | | | | | | Std |
| | plants | 1 | 3 | 5 | 7 | 9 | mean | Dev |
| Source_K | 15 | 0 | 0 | 0 | 0 | 15 | 9 | 0 |
| Inbred IR | 20 | 0 | 0 | 0 | 0 | 20 | 9 | 0 |
| Haz Tm-S | 20 | 20 | 0 | 0 | 0 | 0 | 1 | 0 |
| Haz Tm-R | 14 | 6 | 8 | 0 | 0 | 0 | 2.1429 | 1.02711 |
| Healthy control | 5 | 0 | 0 | 0 | 0 | 5 | 9 | 0 |

Table 12 reports the average number of local lesions on Tobacco cv. Xanthii plants.

TABLE 12
ToBRFV Virus presence detection by bioassay on Tobacco
cv. Xanthii (167 days after inoculation).

| | | Average | | | |
|---|---|---|---|---|---|
| | | nb of | | | |
| | | local | | Nb of | Nb of |
| | Nb | lesions on | | “positive” | “negative” |
| Name | of plants | “xanthii” | Std Dev | plants | plants |
| Haz Tm-S | 2 | >400 | 0 | 2 | 0 |
| Healthy control | 2 | 0 | 0 | 0 | 2 |
| Source_K | 15 | 0 | 0 | 0 | 15 |
| Inbred IR | 2 | >400 | 0 | 2 | 0 |

Conclusion of the trial: Source_K is resistant to ToBRFV at the mature plant stage. Plants were resistant at the phenotypic level, and the ELISA result at the final stage of the trial shows almost no presence of the ToBRFV virus (as illustrated on FIG. 6). Bioassays on tobacco confirmed that ToBRFV is either absent or not viable in source K plants after 5 months of experiment.

### Example 6: Characterization of the Resistance Level of Plants Cumulating Source K Resistance and QTL3

300 BC1F2 plants obtained by selfing BC1 described in example 2 were genotyped with markers linked to Tm-1 gene, QTL8 as identified above, and QTL3 as disclosed in the art as providing ToBRFV resistance or tolerance.

### Seed Deposit

One population of BC1F2, named “BC1-132”, comprising the introgressed sequences from Source K, at least from the position corresponding to TO-0039023 to the position corresponding to TO-0202974 on chromosome 8, were used for making a seed deposit at the NCIMB, on 20 Feb. 2023, under accession number NCIMB 44125. QTL8 is segregating in this seed lot, as checked inter alia by the presence of the resistant allele of markers TO-0202968, TO-0202970, TO-0202972 et TO-0202974.

These seeds also comprise the QTL3, as disclosed in WO2018/219941, homozygously, as checked by the presence of the resistant allele of marker TO-0202544. Tm-1 is also segregating in this seed lot.

### Resistance Level

Two repetitions of 9 plants of each haplotype described in table 13, the parents source_K and Elite 1, a line carrying only a QTL8 and a susceptible control Momor were evaluated for resistance to the 2 strains of ToBRFV ToBRFV-H211 and ToBRFV-H3i1, by phenotypic evaluation (bioassay) and by virus quantification by qPCR using the protocols described in examples 2, and 3, especially at the plantlet stage of development.

TABLE 13
haplotypes tested.

| plant | Tm-1 | QTL8 | QTL3 | Comments |
|---|---|---|---|---|
| a | SS | RS | RR | QTL8 hetero; |
| | | | | QTL11 or QTL3 homo |
| c | SS | RS | RS | QTL8 hetero; |
| | | | | QTL11 or QTL3 hetero |
| b | SS | RR | RR | QTL8 homo; |
| | | | | QTL11 or QTL3 homo |
| d | SS | RR | RS | QTL8 homo; |
| | | | | QTL11 or QTL3 hetero |
| n | SS | RS | SS | Control: |
| | | | | QTL8 hetero only |
| m | SS | RR | SS | Control: |
| | | | | QTL8 homo only |
| o | SS | SS | RR | Control: |
| | | | | QTL11 or or QTL3 homo only |
| Source K | RR | RR | SS | Control |
| Line QTL8 | SS | RR | SS | Control |
| Elite 1 | SS | SS | RR | Control |
| S control | SS | SS | SS | control |

For the genotype, “R” means resistant allele and “S” susceptible allele, irrespective of whether the Resistant allele is recessive or dominant.

For each isolate, two repetitions of 9 plants were evaluated.

This assay aims at demonstrating the enhanced resistance of plants cumulating QTL8 and QTL3, irrespective of the infecting strain, even when infection occurs at the plantlet stage.

### Example 7: Source K+QTL3 Spectrum of Resistance to Several ToBRFV Isolates

Two bioassays were performed, in different locations, to evaluate the efficiency of resistance of source_K (Tm-1+QTL8) or plants of the invention (HAPLO a to HAPLO o in table 13) against 6 isolates of ToBRFV representing the pathogen diversity. One bioassay was performed using 3 different ToBRFV strains, from different geographical areas (ToBRFV-H212, ToBRFV-H112, ToBRFV-H312). The second bioassay was performed using 3 ToBRFV isolates, including ToBRFV-H2i3, ToBRFV-H1i3, ToBRFV-H3i3.

The protocols described previously (examples 2 and 3) for bioassays and qPCR were used.

This assay aims at demonstrating the enhanced resistance of plants cumulating QTL8 and QTL3, irrespective of the infecting strain. It is indeed known that different strains of ToBRFV are circulating, with different levels of aggressivity; especially the strains from ToBRFV-H3 pathotype are known to be more aggressive than the ToBRFV-H1 and ToBRFV-H2 pathotypes.

### Example 8: High Temperature Evaluation of Resistance of Source K and BC1F2 Combining QTL8 and QTL3 or QTL11

Plants carrying the 2 QTLs of resistance (QTL8 and QTL3 or QTL11) and/or potentially the Tm-1 gene, plants of source_K and the susceptible control Momor were evaluated for resistance to ToBRFV at higher temperature. 32 combinations of genotypes (“Combos”) were tested in 1-8 repeats of 5 plants each, in a field tunnel trial in Israel. Same protocol than the one described in example 2. was used except that plants were kept at higher temperatures after mechanical inoculation.

This assay aims at demonstrating the enhanced resistance of plants cumulating QTL8 and QTL3/QTL11, even at high temperatures.

### Method & Timetable.

Mechanical inoculation: one month after sowing.
Transplanting: day after mechanical inoculation.
1st Foliar symptoms scoring: around 7 weeks after transplantation.
Sampling for 1st Bioassay: around 8 weeks after transplantation.
2nd (final) foliar symptoms scoring: around 3 months after inoculation and transplantation.
Sampling for 2nd Bioassay: around 3.5 months after inoculation and transplantation;
Foliar symptoms scoring scale is 1-9 (1=most severe symptoms; 9=no symptoms)
Climatic data: temperature and relative humidity data inside the field tunnel was recorded with a “HOBO” placed at the center of the tunnel at a height of ca. 1.5 meter above the surface.
Bioassay: for most of the “Combos” which were tested by bioassay 3 plots were sampled, for each plot 3 plants were sampled—1 leaflet for each plant. These 3 leaflets were bulked into one ‘BIOREBA” bag. Each bag was tested by mechanical inoculation of 4 leaves of tobacco cv. “Xanthi” (2 “Xanthi” plants×2 leaves in each plant).

### Results on Climatic Data

### 1^{st} Period of the Trial: End of June to Beginning of August

Relative humidity daily pattern is quite similar during the entire period: the highest humidity is at the early morning hours (ca. 80%, around 05:00-07:00), the lowest humidity is at noon hours (15% and lower, around 10:00-16:00).
Temperature in each of the days during the data collection period exceeded 43° C.
Overall—at the 1^{st} period, 29% of the entire time—temperature exceeded 40° C. (in average—in each day the temp. exceeded 40° C. for 7 hours).
15% of the entire time—temperature exceeded 45° C. (in average—in each day the temp. exceeded 45° C. for 3.6 hours).
The maximal temperature: 51.2° C.

### 2^{nd} Period of the Trial: Mid-August to Beginning of October

Until mid-September, the temperature remained at the same level and even higher as in the 1st period, only at the last 3 weeks of the trial (13.9-5.10) the heat level decreased.
The maximal temperature: 55.1° C. . . .
Overall—at the 2^{nd} period, 21% of the entire time—temperature exceeded 40° C.
8% of the entire time—temperature exceeded 45° C.

### Conclusions and Remarks Regarding Evaluation of Resistance

1. In general, the trial was transplanted at a late date which is normally not used for transplanting of tomatoes in this region of Israel, because too hot. As a result, at the first scoring-14% of the plants which were transplanted (143 out of 1013) died. Additional 21% (216 plants) were problematic for evaluation (too chlorotic, curling of the foliage, distorted foliage, or too small plants).
2. The climatic data of the first half of the season shows severe hot temperature pressure, which renders the results on the plants difficult to analyze, some genotypes having less than 12 plants. The climatic data of the second half of the season shows that most of this second half of the season was as hot as the first half. Only at the last 3 weeks of the trial the temperature started to decrease.
3. Phenotypic and bioassay (virus content) data:
   In view of the number of dead plants or which were problematic, further to the hot climatic conditions, there were not sufficient plants to obtain reliable results for some genotypes (combinations of genetic determinants). The bioassays were carried out preferentially on plants corresponding to homozygous combinations for the genetic determinants of interest.

When at least one ‘BIOREBA” bag, for a given genotype, gives positive results regarding presence of the virus, the corresponding genotype (combination of genetic determinants) was considered as positive regarding the presence of the virus.

The results of the phenotypic scoring and second bioassay are given in the tables 14A (Combinations of homozygous genotypes), 14B (independently of the Tm-1 effect) and 14C (haplotypes with less than 12 plants were removed as statistically not representative) below. Grouping of the results in Table 14A, 14B and 14C was achieved by applying Tukey test.

TABLE 14A
Combinations comprising homozygous genotypes.

| | | | | | | | | LSM (leaf | virus presence |
|---|---|---|---|---|---|---|---|---|---|
| | TM1 | QTL8 | QTL11 | TUKEY TEST | | | | score) | (2nd bioassay) |
| Combo | SS | RR | RR | A | | | | 9 | NO |
| | RR | RR | SS | A | | | | 8.95 | NO |
| | RR | RR | RR | A | B | | | 8.14 | NO |
| | SS | SS | RR | A | B | C | | 7.47 | YES |
| | SS | RR | SS | | B | C | | 6.23 | YES |
| | RR | SS | SS | | | C | | 5.59 | YES |
| | SS | SS | SS | | | | D | 2.76 | YES |

TABLE 14B
Combinations of genotypes, irrespective of the
Tm-1 presence, with QTL11e (corresponding to
“QTL3”) and QTL11d (corresponding to “QTL11”).

| | | | | | Least Squares |
|---|---|---|---|---|---|
| QTL combination | Tuckey test | | | | means on leaf score |
| QTL8 (RS), QTL11e (RS) | A | | | | 9 |
| QTL8 (RS), QTL11d (RR) | A | | | | 8.9 |
| QTL8 (RR), QTL11d (RR) | A | | | | 8.7 |
| QTL8 (RR), QTL11d (RS) | A | | | | 8.7 |
| QTL8 (RR), QTL11 (SS) | A | | | | 8.6 |
| QTL8 (SS), QTL11e (RR) | A | B | | | 8 |
| QTL8 (RS), QTL11 (SS) | | B | C | | 7.9 |
| QTL8 (SS), QTL11e (RS) | | | C | D | 6.1 |
| QTL8 (SS), QTL11 (SS) | | | | D | 4.3 |

TABLE 14C
Combinations comprising homozygous and heterozygous genotypes,
irrespective of the type of QTL11, where the genotypes with less
than 12 plants have been removed for statistical reasons.

| Haplotype | | | | | | | | | | | Least Squares means on |
|---|---|---|---|---|---|---|---|---|---|---|---|
| QTL8 | TM1 | QTL11 | Tuckey test rank | | | | | | | | leaf score |
| RS | SS | RR | A | | | | | | | | 8.9345011 |
| RR | RS | RS | A | | | | | | | | 8.9225575 |
| RS | RR | RS | A | | | | | | | | 8.8781019 |
| RR | RS | RR | A | | | | | | | | 8.8686789 |
| RR | RR | RS | A | | | | | | | | 8.8313838 |
| RS | RR | RR | A | | | | | | | | 8.6783614 |
| RS | RS | RR | A | | | | | | | | 8.4506454 |
| RR | RR | SS | A | | | | | | | | 8.3751617 |
| RR | RS | SS | A | B | | | | | | | 7.9026765 |
| RR | SS | SS | A | B | | | | | | | 7.6709089 |
| RS | RR | SS | | B | C | D | E | | | | 6.5876829 |
| RS | RS | SS | | | C | D | E | | | | 6.2966394 |
| RS | SS | SS | | | | D | E | F | | | 5.4741108 |
| SS | RR | SS | | | | | | F | G | | 4.2156028 |
| SS | RS | SS | | | | | | | G | | 4.1863369 |
| SS | SS | SS | | | | | | | | H | 2.9990997 |

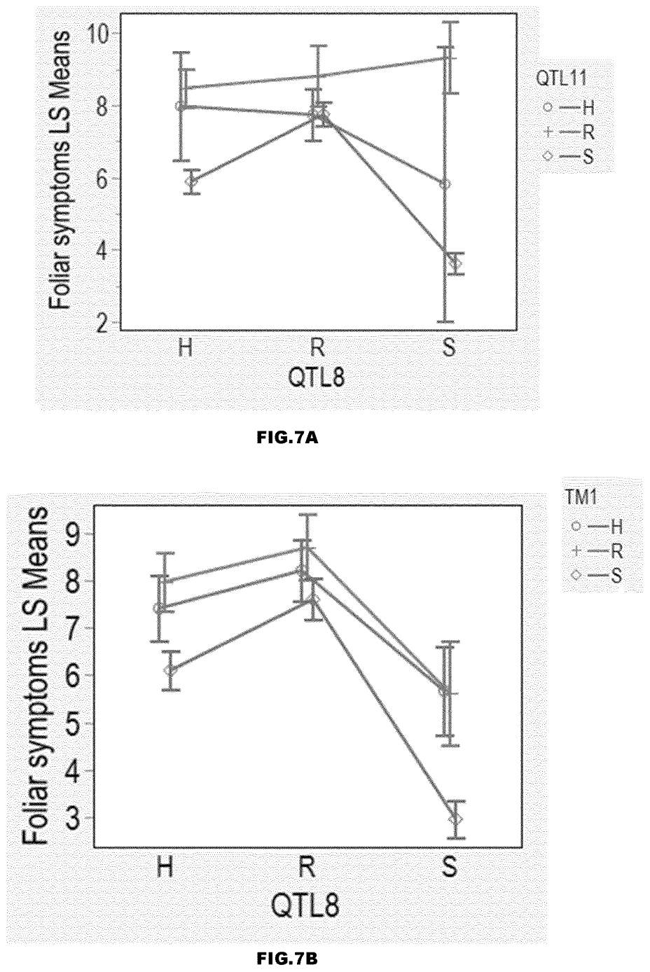

FIG. 7A illustrates the effect of the combination of QTL8 and QTL11 and demonstrates that the foliar symptoms of the plants are improved when QTL8, either homozygous or heterozygous, is combined with QTL11 (Corresponding to either “QTL3” or “QTL11”). FIG. 7B illustrates the same results for the combination of QTL8 and Tm-1.
Source K itself—as in previous trials, most of the plants are symptomless, except a very few plants (2 out of 29 at the 1^{st} scoring, 4 out of 29 at the 2^{nd} scoring) which show ToBRFV foliar symptoms. In accordance with the phenotypic data, the 2^{nd} bioassay results show that symptomless source K plants contain no virus.
QTL-8—when it is the only resistance gene, most of the plants are symptomless but some shows symptoms. Similarly, the 2^{nd} bioassay shows that some of the plants comprising the QTL-8 alone, does contain virus. It is noted that the results of the 2^{nd} bioassay were particularly severe, in view of the late assessment, and the very hot season.
The combination of QTL-8 with Tm-1 (at both at the homozygotic and heterozygotic state) improve the resistance level (=less symptomatic plants). The 2^{nd} bioassay results show that at the 24 plants of Combos having QTL-8-R and Tm-1R-no virus content is detected.

These results illustrate the enhanced resistance, especially at high and even very high temperatures, of plants comprising the QTL8, either homozygously or heterozygously, in combination either with a QTL on chromosome 11 (homozygously or heterozygously) or with the Tm-1 gene (homozygously or heterozygously); the resistance being clearly improved at the leaf symptoms level, especially when considered in combination with the presence of viral sequences.

### Example 9: Tests with Different QTL on Chromosome 11

Plants comprising the QTL8 from Source_K, in association with the QTL11 as disclosed in WO2021/245282 were produced. Their resistance against ToBRFV infection was tested according to the protocols disclosed in the previous examples, especially by qPCR and scoring of foliar symptoms, at 28 days post inoculation, and compared with the results obtained with the QTL3 as disclosed in WO2018/219941 and present in Elite 1. All the tested plants are susceptible with regard to Tm-1.

This assay aims at demonstrating that the enhanced resistance obtained when cumulating the QTL8 of Souce_K, with a QTL on chromosome 11 providing leaf resistance to ToBRFV and reduced viral replication, is not limited to QTL3, but is shared by QTL11 as well, providing ToBRFV resistance, and more specifically ToBRFV leaf resistance or tolerance.

The test was conducted using an artificial bioassay at seedling stage, with an aggressive strain of ToBRFV (ToBRFV-H3i1). The temperature was around 25° C.

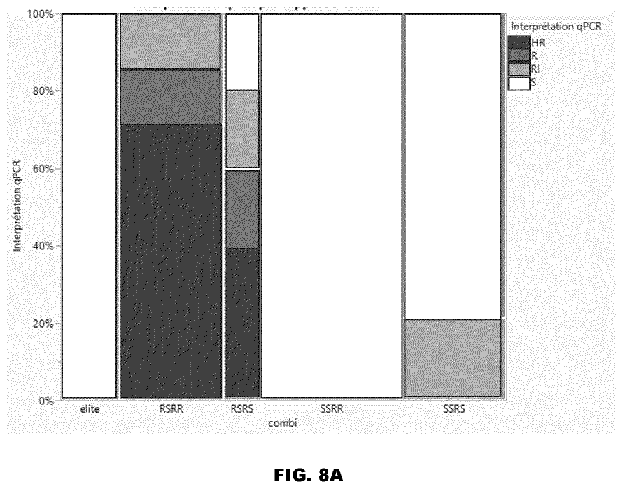

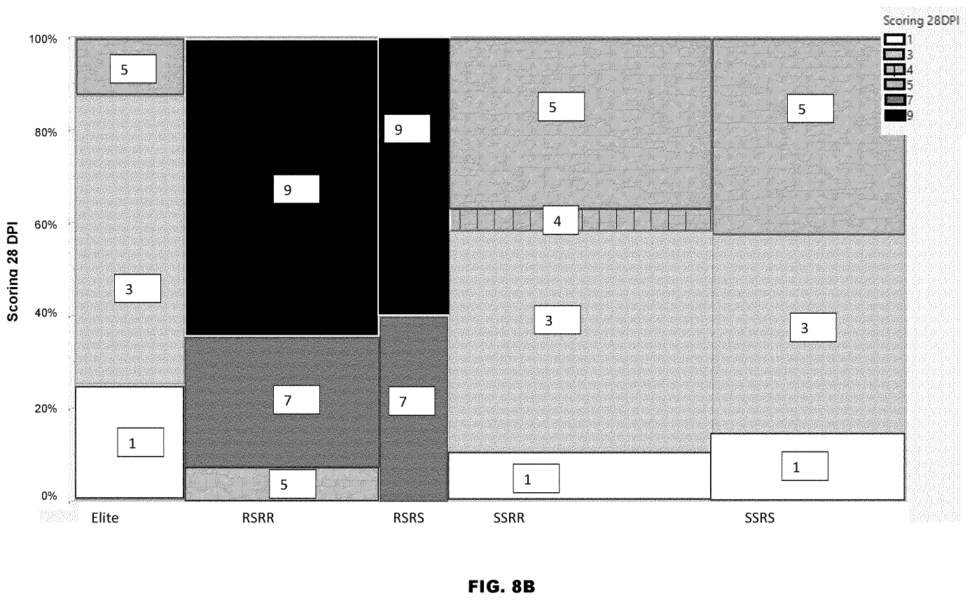

The results are illustrated in FIG. 8A (qPCR) and 8B (foliar symptoms). They show that QTL8, when associated with QTL11, greatly reduces or even abrogates the viral replication (FIG. 8A), as was observed for the combination of QTL8 with QTL3, and that 93% of the plants bearing QTL8 and QTL11 are without symptoms, and 7% are intermediate resistant. All the plants only comprising QTL11 are with symptoms, in the tested conditions i.e. in an artificial bioassay with a strain of ToBRFV known as being an aggressive strain.

### Example 10: Tests with Additional QTLs

Plants cumulating the QTL8 and QTL11 or QTL3 as described in the preceding examples, and also comprising either the Tm-1 gene, homozygously or heterozygously, or a QTL9, as disclosed in WO2021/245282, homozygously or heterozygously, or both, were produced. Their resistance against ToBRFV infection was tested, using the protocols described in the previous examples, and their resistance level was compared to the resistance of plants only comprising the combination of QTL8 and QTL3 or QTL11, without the additional QTLs.

This assay aims at demonstrating that the further combination with a QTL9 as defined in the literature, or Tm-1, has a positive effect on the level of ToBRFV resistance.

### Example 11 Genetic Modification of Tomato Seeds by Ethyl Methane Sulfonate (EMS)

Seeds of a tomato varieties are to be treated with EMS by submergence of approximately 2000 seeds per variety into an aerated solution of either 0.5% (w/V) or 0.7% EMS for 24 hours at room temperature. Approximately 1500 treated seeds per variety per EMS dose are germinated and the resulting plants are grown, preferably in a greenhouse, for example, from May to September, to produce seeds.

Following maturation, M2 seeds are harvested and bulked in one pool per variety per treatment. The resulting pools of M2 seeds are used as starting material to identify the individual M2 seeds and the plants with a resistance to Tomato Brown Rugose Fruit virus.

### Example 12: Evaluation of Resistance of Source K and Plants Combining QTL8 and QTL3 or QTL11 and/or Tm-1 Under Multi-Virus Infection

Bioassays were performed with the protocol described in section 1.2 to evaluate the efficiency of resistance of source_K (Tm-1+QTL8) or plants of the invention (HAPLO a to HAPLO o in table 13) against 3 isolates of ToBRFV representing the pathogen diversity in the context of multi-virus infection. Plants were inoculated with a mix of two or 3 virus, including ToBRFV and PepMV. Momor (comprising the Tm2-2 gene) was used as control.

The protocols described previously (examples 2 and 3) for bioassays and qPCR were used, and for scoring of foliar symptoms.

This assay aims at demonstrating the efficiency resistance to ToBRFV of plants cumulating QTL8, QTL3 or QTL11, even when inoculated with another virus.

Partial results are presented in the table 15 below:

TABLE 15

| Momor (Tm2-2) | | QTL8 + Tm-1 | | Absence of QTL8 | |
|---|---|---|---|---|---|
| | Indexing on | | Indexing on | | Indexing on |
| Foliar | tobacco | Foliar | tobacco | Foliar | tobacco |
| scoring | (3 plants) | scoring | (3 plants) | scoring | (3 plants) |
| 3 | Many spots of | 9 | No necrosis | 7 | necrosis |
| | necrosis | | | | |
| 1 | Many spots of | 8.3 | No necrosis | 5 | necrosis |
| | necrosis | | | | |

### LIST OF REFERENCES

Gao et al, 2016. DNA-guided genome editing using the Natronobacterium gregoryi Argonaute. Nat Biotechnol 34, 768-773.
Ishibashi et al, 2007. An inhibitor of viral RNA replication is encoded by a plant resistance gene. PNAS Aug. 21, 2007 104 (34) 13833-13838.
Jewehan at al, 2022. Evaluation of responses to tomato brown rugose fruit virus (ToBRFV) and selection of resistant lines in Solanum habrochaites and Solanum peruvianum germplasm. J Gen Plant Pathol 88, 187-196.
Luria et al, 2017. A New Israeli Tobamovirus Isolate Infects Tomato Plants Harboring Tm-22 Resistance Genes. PLOS One. 2017 Jan. 20; 12(1):e0170429.
Mascia et al 2010. Evaluation of reference genes for quantitative reverse-transcription polymerase chain reaction normalization in infected tomato plants. Mol Plant Pathol. 2010 November; 11(6):805-16.
Nagai et al, 2019. Signaling pathway played by salicylic acid, gentisic acid, nitric oxide, polyamines and non-enzymatic antioxidants in compatible and incompatible Solanum-tomato mottle mosaic virus interactions. Plant Sci. 2020 January; 290:110274.
Pelham et al, 1970. The establishment of a new strain of tobacco mosaic virus resulting from the use of resistant varieties of tomato.; Ann. Appl. Biol., 65:293-297.
Pfaffl M W, 2001. A new mathematical model for relative quantification in real-time RT-PCR. Nucleic Acids Res.; 29(9):e45.
Salem et al. 2016. A new tobamovirus infecting tomato crops in Jordan. Arch Virol. 2016 February; 161(2):503-6.
Seong, K. et al, 2022. A draft genome assembly for the heterozygous wild tomato Solanum habrochaites highlights haplotypic structural variations of intracellular immune receptors. bioRxiv.
Stevens, M., and Rick, C. M. 1986. Genetics and breeding.
Sui et al, 2017. Molecular and Biological Characterization of Tomato mottle mosaic virus and Development of RT-PCR Detection. Plant Dis. 2017 May; 101(5):704-711.

1. A Solanum lycopersicum plant resistant to Tomato Brown Rugose Fruit virus (ToBRFV) comprising in its genome the combination of:
a. a resistance quantitative trait locus (QTL) on chromosome 8 conferring resistance to ToBRFV, and
b. at least one QTL on chromosome 11, conferring to the plant foliar resistance to ToBRFV when present homozygously, or the Tm-1 gene,
wherein the plant has an improved resistance with respect to the resistance conferred by the QTL on chromosome 8 or 11 alone,
and wherein the resistance QTL on chromosome 8 is introgressed from S. habrochaites.

2. The S. lycopersicum plant according to claim 1, wherein the QTL on chromosome 8 is obtainable from a S. lycopersicum plant grown from seeds of LVSTBRFVRES3, representative seeds of which were deposited with the NCIMB under accession number NCIMB 44125, by crossing and selection of resistant plants.

3. The S. lycopersicum plant according to claim 1, wherein the introgressed sequences from S. habrochaites on chromosome 8 are present between position 59100098 and 61782030, according to genome assembly SL3.0 on chromosome 8.

4. The S. lycopersicum plant according to claim 1, wherein the resistance QTL on chromosome 8 comprises at least one of: allele G of SNP marker TO-0202968, allele A of TO-0202572, allele A of SNP marker TO-0202970, allele A of SNP marker TO-0202972 and allele A of SNP marker TO-0202974.

5. A Solanum lycopersicum plant resistant to ToBRFV comprising in its genome the combination of:
c. a resistance gene on chromosome 8 coding for a resistance protein having at least 90% sequence identity with SEQ ID NO.1, conferring resistance to ToBRFV, and
d. at least one quantitative trait locus (QTL) on chromosome 11, conferring to the plant foliar resistance to ToBRFV when present homozygously, or the Tm-1 gene,
wherein the plant has an improved resistance with respect to the resistance conferred by one of the resistance gene or the QTL on chromosome 11 alone.

6. The S. lycopersicum plant according to claim 5, wherein the resistance gene is present on chromosome 8 in the genome of seeds of LVSTBRFVRES3, representative seeds of which were deposited with the NCIMB under accession number NCIMB 44125.

7. The S. lycopersicum plant according to claim 1, wherein the QTL on chromosome 11 is chosen from:
QTL3, present in the genome of a plant of the seeds HAZTBRFVRES1 NCIMB accession number 42758, and of the seeds LVSTBRFVRES3 NCIMB accession number 44125 and
QTL11, introgressed from S. pimpinellifolium and present in the genome of a plant of the seeds LVSTBRFVRES2 NCIMB accession number 43591.

8. The S. lycopersicum plant according to claim 1, further comprising the Tm-1 gene, on chromosome 2, homozygously or heterozygously.

9. The S. lycopersicum plant according to claim 1, wherein the resistance QTL or resistance gene on chromosome 8 is present heterozygously or homozygously.

10. The S. lycopersicum plant according to claim 1, wherein the QTL on chromosome 11 is present homozygously or heterozygously.

11. (canceled)

12. The S. lycopersicum plant according to claim 1, wherein the plant delays, reduces or inhibits the replication or multiplication of the virus, in the absence of infection symptoms, even at temperature of 28° C. or above.

13. (canceled)

14. The S. lycopersicum plant according to claim 1, comprising one of the following combinations:
a) the resistance gene or QTL on chromosome 8 heterozygously, and QTL on chromosome 11 homozygously;
b) the resistance gene or QTL on chromosome 8 homozygously, and QTL on chromosome 11 homozygously;
c) the resistance gene or QTL on chromosome 8 heterozygously, and Tm-1 on chromosome 2 heterozygously;
d) the resistance gene or QTL on chromosome 8 homozygously, and Tm-1 on chromosome 2 heterozygously;
e) the resistance gene or QTL on chromosome 8 heterozygously, and Tm-1 on chromosome 2 homozygously;
f) the resistance gene or QTL on chromosome 8 homozygously, and Tm-1 on chromosome 2 homozygously;
g) the resistance gene or QTL on chromosome 8 heterozygously, Tm-1 heterozygously and QTL on chromosome 11 homozygously;
h) the resistance gene or QTL on chromosome 8 homozygously, Tm-1 heterozygously and QTL on chromosome 11 homozygously;
i) the resistance gene or QTL on chromosome 8 heterozygously, Tm-1 homozygously and QTL on chromosome 11 homozygously; and
j) the resistance gene or QTL on chromosome 8 homozygously, Tm-1 homozygously and QTL on chromosome 11 homozygously.

15. The S. lycopersicum plant according to claim 1, further comprising the Tm-2 resistance gene, on chromosome 9.

16. The S. lycopersicum plant according to claim 1, wherein the QTL, on chromosome 11, is to be found within the chromosomal region delimited by TO-0122252 (SEQ ID NO.6) and TO-0162427 (SEQ ID NO.7) for QTL3, or within the chromosomal region delimited by the SNP TO-0201237 (SEQ ID NO.3) and the marker SL2.50ch11_9924232 (SEQ ID NO.4).

17-20. (canceled)

21. A cell of a S. lycopersicum plant according to claim 1, comprising in its genome the resistance QTL or gene on chromosome 8 and the at least one QTL on chromosome 11, independently conferring resistance to ToBRFV.

22. A tissue culture of cells of the plant according to claim 1, wherein the cells are derived from embryos, protoplasts, meristematic cells, callus, pollen, leaves, anthers, stems, petioles, roots, root tips, seeds, flowers, cotyledons, and/or hypocotyls, and contain in their genome the resistance QTL or gene on chromosome 8 and the at least one QTL on chromosome 11, independently conferring the resistance to ToBRFV.

23. A plant part of a S. lycopersicum plant according to claim 1, wherein said plant part comprises cells comprising in their genome the resistance QTL or gene on chromosome 8 and the at least one QTL on chromosome 11, independently conferring resistance to ToBRFV.

24. Seed of a S. lycopersicum plant, which develops into a plant according to claim 1 or which derives from a plant according to claim 1.

25. A method for conferring improved resistance to ToBRFV to S. lycopersicum plants, comprising the steps of:
a) crossing a S. lycopersicum plant bearing a QTL on chromosome 11, conferring independently ToBRFV resistance, and/or bearing the Tm-1 gene, with a S. lycopersicum plant comprising a resistance QTL on chromosome 8, wherein the resistance QTL confers ToBRFV resistance,
b) selecting a plant in the progeny thus obtained, bearing the QTL on chromosome 11 and/or the Tm-1 gene, and the resistance QTL on chromosome 8;
c) optionally self-pollinating one or several times the plant obtained at step b) and selecting in the progeny thus obtained a plant having an improved resistance to ToBRFV,
wherein the resistance QTL on chromosome 8 is introgressed from S. habrochaites.

26-34. (canceled)

35. A method of producing tomatoes comprising:
a. growing a S. lycopersicum plant according to claim 1;
b. allowing the plant to set fruit; and
harvesting fruit of the plant.


SEQUENCE LISTING (60 sequences)

SEQ ID NO: 1 - protein, 976 aa, Solanum habrochaites
MAEAFLQIMLENLTCFIQGELGLILGFKDEFEKLQSTFTTIQAVVQDAQLKQLKDKAIENWLQKLNGAAYEADDILDECKTEAPIIQKKNKYGCYHPNVITFRHKIGKRMKKIMEKLDAIAAERIKFHLDERTIERQVATRQTGFVLNEPQVYGRDKDKDEIVKILINNAQTLSVLPILGMGGLGKTTLAQMVFNDQRVIEHFHPKIWICVSEDFNEKRLIKEIVESIEEKSLGDMDLAPLQKKLQDLLNGKKYLLVLDDVWNEDQDKWAKLRQVLKAGASGAYVLTTTRLEKVGSIMGALQPYELSNLSQEDCWLLFMQRAFGHQEEMNLNLVAIGKVIVKKCGGVPLAAKTLGGILRFKREERQWEHVRDNEIWSLPQDESSILPSLRLSYHHLPLDLRQCFAYCAVFPKDTKMIKENLITLWMAHGFLLSKGNLELEDVGNEVWNELYLRCFFQEIEAKSGNTYFKIHDLIHDLATSLFSASTSSSNIREINVEGYLHMMSIGFAKVVSSYSPPHLQKFVSLRVLNLSSMGLKQLPSSIGDLVHLRYLNLSLNNMRTLPKQLCKLQNLQTLNVEYCWSLCCLPKETSKLGSLRNLLLDGCDGLDSMPPRIGSLTCLKTLSFFVIGERKDSLLGELRNLNLYGSIEITHLERVKNDRDAKEANLSAKENLHSLSMRWKGPHRYESEEVEVLESLKPHPNLTSLLITGFRGFRLPKWMNHSVLKNVVSIAIRGCENCSCLPPFGDLPCLESLELGDGSAELEYVEDSGFPTRRRFPSLRKLIIVNFDNLKGLLKEAGEEQFPVLEEMTISWCPVLVIPTLSSVKKLVVYRNMSDAIGLRSIYNLRALTSLNISHNLTATSLPEEMFKSLANLKYLEISFIFNLKELPNSLASLNALKHLKIEYCDALESLPEEGVKGLTSLTELSITNCKRLKCLPEGLQHLTNLSVRECPTLAKRCEKGIGQDWYKIAHIPHLL

SEQ ID NO: 2 - DNA, 2928 bp, Solanum habrochaites
  source 1..2928: mol_type = unassigned DNA
atggctgaagctttccttcaaattatgttagagaatctgacttgtttcatccaaggggaacttggattgattcttggttttaaggatgagttcgaaaagcttcaaagcacgtttactacaatccaagctgtggtacaagatgctcagttgaagcaattgaaggacaaggcaattgaaaattggttgcagaaactcaatggtgctgcatatgaagctgatgacatcttggacgaatgtaaaactgaggcaccaattatacagaagaagaataaatatgggtgttatcatccaaacgttatcactttccgtcacaagattgggaaaaggatgaaaaagattatggagaaactagatgcaattgcagcggaacgaattaagtttcatttggatgaaaggactatagagagacaagttgctacacgccaaacaggttttgttttaaatgaaccacaagtttatggaagagacaaagataaggatgagatagtgaaaatcctgataaacaatgcccaaacactttcagtcctcccaatacttggtatggggggactaggaaagacgacccttgcccaaatggtcttcaatgatcagagagtaattgaacatttccatcccaaaatatggatttgtgtctcggaagattttaatgaaaagaggttgataaaggaaattgtagaatctattgaagaaaagtcacttggtgacatggacttggctccacttcaaaagaagcttcaggacttgctgaatggaaaaaaatatttgcttgtcttagatgatgtttggaatgaagatcaagataagtgggctaagttaagacaagtcttgaaggctggagcaagtggtgcttatgttctaaccactacccgtcttgaaaaggttggatcaatcatgggggcattgcaaccatatgaattgtcaaatttgtctcaagaagattgttggttgttgttcatgcaacgtgcatttgggcaccaagaagaaatgaatcttaatctagtggctatcggaaaggtgattgtgaaaaaatgtggtggtgtgcctctagcagctaaaactcttggaggtattttgcgcttcaagagagaagaaagacagtgggaacatgtgagagataatgagatttggagtctgcctcaagatgaaagttctattttgccttctctaagactgagctatcatcaccttccacttgatttgagacaatgttttgcgtattgtgcagtattcccaaaggacaccaaaatgataaaggaaaatctcatcactctctggatggcacatggttttcttttatcaaagggaaacttggagctagaggatgtgggtaatgaagtatggaatgaattatacttgaggtgtttcttccaagagattgaagctaaatccggtaatacttatttcaagatacatgatctcattcatgatttggcaacatctctattttcagcaagcacatcaagcagcaatatccgagaaataaatgtagaaggttacctacatatgatgtcgattggtttcgcaaaagtggtgtcttcttactctcctcctcacttgcaaaagtttgtctcattgagggttcttaatctaagttccatgggacttaagcagttaccgtcctccattggagatctagtacatttaagatacttgaacctctctctcaataacatgcgtactcttccaaagcagttatgcaagcttcaaaatctgcagactcttaatgtagagtattgctggtcactttgttgtttgccaaaagaaacaagtaaacttggtagtctccgaaatctcttacttgatggttgcgatggattggattctatgccaccaaggataggatctttgacatgccttaagactctaagtttctttgttattggcgagagaaaagattctctacttggtgaattacgaaacctgaatttgtatgggtcaattgaaatcacgcatcttgagagagtgaagaatgatagggatgcaaaagaagccaatttatctgcaaaagaaaatctgcattctttaagcatgagatggaaaggaccacatagatatgaatcagaagaagttgaagtgcttgaatccctcaaaccacaccctaatttgacttctttactaatcactggcttcagaggattccgtcttccaaagtggatgaatcactcagttttgaaaaatgttgtctctattgcaattagaggttgtgaaaactgctcatgcttaccaccgtttggtgatctgccttgtcttgaaagtctagagttaggagatgggtctgcggaactggagtatgttgaagattctggattccctacaagaagaaggtttccatctctgagaaaacttattatagtcaattttgataatctgaaaggattgctgaaagaggcaggagaagagcaattccccgtgcttgaagagatgacaattagctggtgtcctgtgcttgttattccgaccctttcttctgtcaagaaattggtagtttatcggaacatgtcagatgcaataggtttgaggtccatatataatcttagggctcttacttccctcaacattagccataacttgacagctacttcgctcccagaagagatgttcaaaagccttgcaaatctcaaatacttggaaatctctttcatcttcaatctcaaagagctgccaaacagcctggctagtctcaatgctttgaagcatctgaaaattgaatattgtgacgcactcgagagtctccccgaggaaggggtgaaaggtttaacttcactcacagaattatccataacaaattgtaagaggctaaaatgtttaccggagggattgcagcacctaacaaatttatcagttagggaatgtccaacactggccaagcggtgtgagaagggaataggacaagactggtacaaaattgctcacattcctcatctgctt

SEQ ID NO: 3 - DNA, 401 bp, Solanum lycopersicum
  misc_feature 201: allele = susceptible : A; resistant : G
  source 1..401: mol_type = unassigned DNA
gttactttaatatttaattgtattataataaattctatatactaagggatgcagttattttataattttgagaaaatacatggcatattcgttcatcttacttgtgaccttttttttaatagattatagtacattccttattaaatgggttaaagctatgatctggaaatctccatgcttaacgtctgtatgttttcaatrctatggtcaaacaatttgctcatataatttgtgtacgtgttatgaatttgggatttttcatatgagtaaaatagtttttaaaaaaaattctgttatgtccttgtgggcataggttcaaccttgaagtaaatttagggatcataataatgggtgttgttaattttgaaatcttattatggtcatttgtttgagtagattgc

SEQ ID NO: 4 - DNA, 301 bp, Solanum lycopersicum
  misc_feature 151: allele = susceptible : G; resistant : GT
  source 1..301: mol_type = unassigned DNA
gcaaaaaaataaaaaataaaaaaacgctagacagattgatgactatttatgctcatttcaaaacataactaatttgtatttaatttttttttaccttttttgattattttttttgctgtatgattttctcggttctattattagttatttgtttttttaaatattaatcataataacataaatacataattaatcccttaacaaaataaaataatgacatttttaataaacaataaaattgttgctaagaagtcccactaaaagcttaaatattgttgttttgaaaaaaatttccccaagg

SEQ ID NO: 5 - DNA, 401 bp, Solanum lycopersicum
  misc_feature 201: allele = susceptible : C; resistant : A
  source 1..401: mol_type = unassigned DNA
tcatcgtagtcagtaagattttaccaaggaattaaaatttattaagtattaagctcattacattagaattagcaccaagatttccaaaaagaactaaattggaattagcaaaaatctgaaatttgaagagttaagttaagtatgagttttgagtcaacttcaaaggaccataaatattagaacacgatgagttagatgtgmtacaagataccatagggaagatattcgaataacctttgcaaagcctttgagtttactaagtttcaagttcatacaagtgagatatgaccttttgaagttaggttctccagttaagcaaagttagccaaaaatagtgaaggtattttggtccttcccttacccaaacagatttaattcgttttagtcatattttagggtta

SEQ ID NO: 6 - DNA, 101 bp, Solanum lycopersicum
  misc_feature 51: allele = susceptible : A; tolerant : T
  source 1..101: mol_type = unassigned DNA
atggcaatagtgaactgcagatacaactgaaattgcagaacacccttaaawatagaatcaatagaaagttgcaacaatatttgaatgatgaagcaacaaag

SEQ ID NO: 7 - DNA, 101 bp, Solanum lycopersicum
  misc_feature 51: allele = susceptible : C; tolerant : T
  source 1..101: mol_type = other DNA
gcaccagttatagtaatgtcctgcttctttcctgtacccttatcagtagcygtgacagaaagaataccgttggtgtcaatgtcgaacttcacttcaatctg

SEQ ID NO: 8 - DNA, 101 bp, Solanum lycopersicum
  misc_feature 51: allele = susceptible : T; tolerant : C
  source 1..101: mol_type = other DNA
agccattgtgattgtgtctgttgtacattaccaaaattctctagagaaagygatacacatgccagccctatcgatataaagcaacgcaaggtggattctgc

SEQ ID NO: 9 - DNA, 101 bp, Solanum lycopersicum
  misc_feature 51: allele = susceptible: C; tolerant : T
  source 1..101: mol_type = unassigned DNA
aacaccaggtagagagcacagcgaaacaatggcctcaggaagatctacttygcgaagtgcagcaagccactccatacctccaccaggctttgatttcagtg

SEQ ID NO: 10 - DNA, 101 bp, Solanum lycopersicum
  misc_feature 51: allele = susceptible : A; tolerant : G
  source 1..101: mol_type = unassigned DNA
tcaactgcaactttaacagctgattcaacttcttcttctttcgaaacatcrcattgaatgtaacgacctccaatagattcagctaaacttgtacctacttc

SEQ ID NO: 11 - DNA, 101 bp, Solanum lycopersicum
  misc_feature 51: allele = susceptible : C; tolerant : A
  source 1..101: mol_type = unassigned DNA
gaggagctatcaacttcatagtcagattcagaaaatgattcagatgaggamgtggctgattcttcttgttttcttttcttccttctgctcgaactctctcc

SEQ ID NO: 12 - DNA, 101 bp, Solanum lycopersicum
  misc_feature 51: allele = susceptible : G; tolerant : A
  source 1..101: mol_type = unassigned DNA
cagaaataatagaaaatcagaaagaaaaatcagctttctaaatggaaaagrcgatggcactatgtttgaagttttaagcaacttttctgaagtcccaaaag

SEQ ID NO: 13 - skipped

SEQ ID NO: 14 - DNA, 101 bp, Solanum lycopersicum
  misc_feature 51: allele = susceptible : A; tolerant : G
  source 1..101: mol_type = unassigned DNA
ctcttggtgacaaaccactggctcaatttcttcgcgaagctaaagctatcrctgatgagcttgtcacggcaggcacacgtgtctcctgatgaattcaatgc

SEQ ID NO: 15 - DNA, 101 bp, Solanum lycopersicum
  misc_feature 51: allele = susceptible : T; tolerant : G
  source 1..101: mol_type = unassigned DNA
cattactgttgagatatctcatcggcaacccctggagcttgcccacccgcktgtcctccaggatctgatttcagaaaggatgaatagtaactgtgtttcag

SEQ ID NO: 16 - DNA, 121 bp, Solanum lycopersicum
  misc_feature 61: allele = susceptible : G; tolerant : A
  source 1..121: mol_type = unassigned DNA
caagaacccaacgacttcttcttctttgcttattgaaaaacttggttttgaaatgaaaggratcgagaaattggatactcagtggttctctactactaaaccttctcctgattttaagaaa

SEQ ID NO: 17 - DNA, 101 bp, Solanum lycopersicum
  source 1..101: mol_type = other DNA
  misc_feature 51: allele = susceptible: T ; tolerant: C
tgatcgacaattcttgttgttgttgaaactctgcaagtgagagagggatgyatatagagaaaggatattggtaaaggacaattctagaagggtctagggaa

SEQ ID NO: 18 - DNA, 401 bp, Solanum lycopersicum
  misc_feature 201: allele = susceptible : T ; resistant : A
  source 1..401: mol_type = unassigned DNA
tgcaacttctattcttccttccgcaggtgggagctgtgtattttatttatcttgtctagttgtactcttattagcttttgtacttgtttaactagatccatgagagttgataaattactttacaagttttaagtaaatgtgtgtctaaagatattatttataaaatcatattattgcaattacttctgtttccttaaaaawtttcacatgtttaattattgggatacgagagttctcttacttaggtgttagagtggtgcctggacggctcggtgggttggatcgtgacataatatatgaatttgtcaaaaaaatttacattatttttgtgatcgctatttcaatataatatacagtaagcatgcatatatgtacaatggatatatttttgtcatgctcta

SEQ ID NO: 19 - DNA, 401 bp, Solanum lycopersicum
  misc_feature 201: allele = susceptible : C; resistant : A
  source 1..401: mol_type = unassigned DNA
tgcgaggcactcgccttgatggttcttactacacttggggcaagtggggtaagtcttggtgcttgaaacacttccctaagacttagattctggtgccctaaccttctgatcatacatgttcttagaggatggaatactagttatcatagttctgctatggggttgaaaacttttcctgactctacgagcgatttccaccamcaaatttctgctaagagtagtcatagttcccagtcctaacctcttattctccttggcttgtttcctaagcttatcaccctcaacctgctgatcatgagtcataagcttaaggatgttcatatctcgtagtaacatagaacttctgcactcagttttcaccaaattttgactccgtacaagaaattattcatctgagccct

SEQ ID NO: 20 - DNA, 401 bp, Solanum lycopersicum
  misc_feature 201: allele = susceptible : C ; resistant : A
  source 1..401: mol_type = unassigned DNA
agtacaaattgcaactttttcaatgttaacgtctgcttttttagtgtataaaaaagaaggattcatgaactcctcagtcatattctattttaaaatacttaaacatcttttttaattatgtctcaagcatgtcaaacatctaatgcaaaaaagacttacgtttgtcgttgtggtaatttagctattttgaggatttcacamaccgatacgaacccagatcgataattatttaattgtgcaattggtgcattgatctttttttaagtggcttgattatgattcatcaacgagcaacccatcaaaattgagtctcggattcgagacatcaaattttcaaagcgttatcaaatttcaaatattctttatctccttattttatctcatgtacaacttgattgact

SEQ ID NO: 21 - DNA, 301 bp, Solanum lycopersicum
  misc_feature 151: allele = susceptible : C; resistant : CT
  source 1..301: mol_type = unassigned DNA
agtgatattgtcttcagctatagaatattatccagcccctctagctttggtcgatttttttttataacaaattttatccaagctatcattgtttttttttttttttcaatttacagcaattgtttcccgactagcctatcaatgttagagcttttttttggtttgaacatcgttgtcttgagaagcttaatccatgttcactgtaggcattgttatttaattttcaattgagtttgttaatctgcctatagatattaatttatttcctcttctttcgatgcatatgatcaatccagtctat

SEQ ID NO: 22 - DNA, 301 bp, Solanum lycopersicum
  misc_feature 151: allele = susceptible : A; resistant : AT
  source 1..301: mol_type = unassigned DNA
acaatgacatgacatcaaaaataatttaatttatcatgtcaaatttgtttaaaaataaattgtaatcgagtgattttaaagaataaagatgttaagttgagtgattttaaagacacaaactaaaatttagtgactttacaagataatttcatatagttcattgaccttttaagatattaactcaaaaaatcaataagtagtcacacacaaaaggtgattgcaaagtaaatttcaatttttagcataagcttagtggggaataccaatgacgaatctaaaagtatcaatacaaaaaatagaa

SEQ ID NO: 23 - DNA, 301 bp, Solanum lycopersicum
  misc_feature 151: allele = susceptible : T; resistant : C
  source 1..301: mol_type = unassigned DNA
aaatgttaatttttttatttataataaatattaatatgtcttagacttgaacttttaagttcattaaagatagttaaatatcgtatttgatatacttaacgaaaaagctaaaacgtaaaagaaaatataataagtttatatcttcttcttytttttttaaatttggatttgtgatagtagggctttgagagaaaattctttttataattttttttttcatattcaaggctcgaacctaagatctatgattaaggaagaagtagcctttccactatacaatacacgttcataatgagttaat

SEQ ID NO: 24 - protein, 754 aa, Solanum lycopersicum
MATAQSNSPRVFCIGTADTKFDELRFLSEHVRSSLNSFSNKSSFKVGVTVVDVSTSWKETNSCADFDFVPSKDVLSCHTLGEETMGTFADIRGLAIAIMSKALETFLSIANDEQNLAGVIGLGGSGGTSLLSSAFRSLPIGIPKVIISTVASGQTESYIGTSDLVLFPSVVDICGINNVSKVVLSNAGAAFAGMVIGRLESSKEHSITNGKFTVGVTMFGVTTPCVNAVKERLVKEGYETLVFHATGVGGRAMEDLVRGGFIQGVLDITTTEVADYVVGGVMACDSSRFDAILEKKIPLVLSVGALDMVNFGPKTTIPPEFQQRKIHEHNEQVSLMRTTVGENKKFAAFIAEKLNKASSSVCVCLPEKGVSALDAPGKDFYDPEATSCLTRELQMLLENNERCQVKVLPYHINDAEFANALVDSFLEISPKSRHVECQPAESKSIQDIQNDNAVLEKYPSCNGKNFSRLNDFPNAKPETLQKRTVILQKLKDQISKGKPIIGAGAGTGISAKFEEAGGVDLIVLYNSGRFRMAGRGSLAGLLPFADANAIVLEMANEVLPVVKEVAVLAGVCATDPFRRMDNFLKQLESVGFCGVQNFPTVGLFDGNFRQNLEETGMGYGLEVEMIAAAHRMGLLTTPYAFCPDEAVAMAEAGADIIVAHMGLTTSGSIGAKTAVSLEESVTCVQAIADATHRIYPDAIVLCHGGPISSPEEAAYVLKRTTGVHGFYGASSMERLPVEQAITATVQQYKSISME

SEQ ID NO: 25 - DNA, 2718 bp, Solanum lycopersicum
  source 1..2718: mol_type = unassigned DNA
gacttttttatttatttaatattaaatattaatattgctccatcggaataaaaataaaaataaaaatggaacctctccacttgacggttgttgccgcctctctgtccaaccgttgccggtgatctttcatcgtcgagattccagaagctaacaactttctaatcttctcaccattctcacactgagttacactctatcgtccattttgaaatctcgattgtaacacgaattgtatcattttgtttgaggaaaatggcaactgcacagagtaattctcctcgagttttctgtatcggaacagctgatacaaaattcgacgagcttcgtttcctctccgagcatgtgagatccagtcttaacagcttctccaataaatcctcattcaaggtaggagtgacagttgttgatgtctcaaccagctggaaggagacaaatagttgtgctgattttgattttgtaccgagtaaggatgtgctgtcatgccatacactaggggaagaaactatgggcacgtttgcagatattagaggcctagctattgcaatcatgagcaaagctcttgaaactttcctaagtatagctaatgatgaacagaatcttgctggagtaattggccttgggggtagtggaggaacatctctattgtcatctgccttccgatctcttccaattgggatcccaaaagttataatatctacagttgccagtggtcaaactgaatcttatattggaacatcagacttggtattgtttccttcagttgtagatatttgtgggattaacaatgtcagtaaggttgttctatctaatgcgggtgcagcatttgctggaatggtgatcgggaggcttgaaagttcaaaagagcatagcatcactaatggaaagtttacagttggtgtaactatgtttggggttacgactccttgtgttaatgctgtcaaagaaagattagtgaaagaaggatatgagactttggtgttccatgccacgggtgtcgggggcagggccatggaggatcttgttagaggaggttttatacagggtgtgctggatattacgacaactgaggttgcagattacgtagttggaggagtaatggcatgtgatagttcccgatttgatgcaatattagagaagaaaattcctttggttctgagtgtgggagcactggatatggtgaattttggtcctaaaactaccatacctcctgagtttcaacaaagaaagatccatgaacataatgagcaggtttccctaatgcgtactacagtaggtgaaaataagaaatttgctgcatttatagcagaaaagttgaacaaggcatcatcaagtgtatgtgtttgcttgccagagaaaggcgtgtctgcattggatgcacccgggaaagacttttatgatcctgaggcaactagttgtcttacacgtgaactacagatgcttcttgaaaataatgaacgttgtcaggttaaggtcctcccttaccatatcaatgatgcggagtttgcaaatgctttagttgattcattcttggaaatctctccgaaatctagacacgtagaatgtcagccagctgagtccaaatctatccaagacattcagaatgataatgctgttctagagaaatatccctcatgcaacgggaaaaacttttctcgcctgaatgactttccaaatgcaaaaccagaaactttgcagaaaagaactgtgatactgcagaaattgaaagatcaaataagtaagggcaagcctattattggggctggtgctggtacaggtatttctgctaagtttgaggaagctggtggtgtagatttgattgtcttgtacaactcagggcgctttaggatggcaggaaggggatccttagctggtctactgccctttgctgatgcaaatgccattgtacttgagatggccaacgaagtattgcctgtggttaaggaagtggcagttctggctggagtttgtgctactgatcctttccgcaggatggacaacttcctgaagcagttggaatccgttggattctgtggggtgcaaaactttccaactgttggtctgtttgacggtaacttcagacaaaatttggaagagactggaatgggttatggcttggaggttgagatgattgcagcagctcacaggatgggccttttgacaaccccatatgctttctgcccagatgaagcagttgctatggcagaagctggtgccgacatcatagttgctcatatggggcttacaacatctggttcaattggtgcaaaaacagccgtctcattggaggaaagtgtaacttgcgttcaagctattgcagatgctactcataggatatatcctgatgcaattgtgctctgccatggaggccctatatcttcccctgaagaagcagcatatgtactgaagagaaccacaggagttcatggattttatggcgcttcaagcatggaaagactaccagttgagcaagctataactgcaactgtccagcagtacaagtctatttctatggagtgagatgcagtggattttggttctttaaatgtccgaacctatatgtatggtcttcacctctttctttatgtatgatcatgtccagggctcctctgtactcatctagcaacttgaaacactacagcactgctgctgcagtttttatctgtccatatgatatcaatttcaacaagttatatcttttcgtcaaaaaaaaaaaaaaaa

SEQ ID NO: 26 - DNA, 20 bp, synthetic construct
  source 1..20: mol_type = other DNA
agatttccctggcttttgga

SEQ ID NO: 27 - DNA, 21 bp, synthetic construct
  source 1..21: mol_type = other DNA
ctctttctgatatcaagcact

SEQ ID NO: 28 - DNA, 22 bp, synthetic construct
  source 1..22: mol_type = other DNA
caaggagagactgctaaatcgg

SEQ ID NO: 29 - DNA, 24 bp, synthetic construct
  source 1..24: mol_type = other DNA
tcgtaaggagtgccctaatgctga

SEQ ID NO: 30 - DNA, 24 bp, synthetic construct
  source 1..24: mol_type = other DNA
caatcgcctccagccttgttgtaa

SEQ ID NO: 31 - DNA, 390 bp, Solanum lycopersicum
  source 1..390: mol_type = unassigned DNA
  misc_feature 297: allele = susceptible : G ; resistant :A
gccctgcccccgacacccgtggcatggaacaccaaagtctcatatccttctttcactaatctttctttgacagcattaacacaaggagtcgtaaccccaaacatagttacaccaactgtaaactttccattagtgatgctatgctcttttgaactttcaagcctnccnatcaccattccagcaaatgctgcacccgcattagatagaacaaccttactnacattgttaatcccacaaatatctacaactgaaggaaacaataccaagtctgatgttccaatataagattcagtttgrccactggcaactgtagatattataacttttgggatcccaattggaagagatcggaaggcagatgacaatagagatgttcctccactaccccca

SEQ ID NO: 32 - DNA, 301 bp, Solanum lycopersicum
  source 1..301: mol_type = unassigned DNA
  misc_feature 151: allele = susceptible : C ; resistant : A
tccccgtgcttgaagagttgacaattaggtgttgtcctgtgtttgttattccgaccctttcttctgtcaagaaattggtagtttatggggacaagtcagatgcaacagttttgaggtccatatataatcttagggctcttacttccctcamcattagccttaactccatagctacttcgctcccagaagagatgttcaaaagccttgcaaattctcaaatacttggaaatctctttcttcgacaatctcaaagagctgccaaacagcctggctagtctcaatgctttgaagcatctggaga

SEQ ID NO: 33 - protein, 979 aa, Solanum habrochaites
MAEAFLQIMLENLTCFIQGELGLILGFKDEFEKLQSTFTTIQAVVQDAQLKQLKDKAIENWLQKLNGAAYEADDILDECKTEAPIIQKKNKYGCYHPNVITFRRKIGKRMKKIMEKLDAIAAERIKFHLDERTIERQVATRQTGFVLNEPQVYGRDKDKDEIVKILINNAQTLSVLPILGMGGLGKTTLAQMVFNDQRVIEHFHPKIWICVSEDFNEKRLIKKIVESIEEKSLGDMDLAPLQKKLQDLLNGKKYLLVLDDVWNEDQDKWAKLRQVLKAGASGAYVLTTTRLEKVGSIMGTLQPYELSNLSQEDCWLLFMQCAFGHQEEMNLNLVAIGKVIVKKCGGVPLAAKTLGGILRFKREERQWEHVRDSEIWNLPQDESSILPALRLSYHHLPLDLRQCFSYCAVFPKDTKMEKENLISLWMAHGFLLSKGNLELEDVGNEVWNELYLRSFFQEIEVQYDRTYFKMHDLIHDLATSLFSASTSSSNIREINVEGYLHMMSIGFAKVVSSYSPPHLQKFVSLRVLNLSSMGLKQLPSSIGDLVHLRYLNLSLNNMRTLPKQLCKLQNLQTLNVEYCWSLCCFPKETSKLGSLRNLLLDGCDGLDSMPPRIGSLTCLKTLSLFVIGERKDSLLGELRNLNLYGSIEITHLERVKNDRDAKEANLSAKENLHSLSMRWEGPHRYESEEVEVLESLKPHSNVTCLTITGFRGIRLPEWMNHSVLKNVVSIAIRGCENCSCLPPFGDLPCLESLELRSGSAEVEYVEDSGFPTRRRFPSMRKLTIENFDNLKGLLKEAGEEQFPVLEELTIRCCPVFVIPTLSSVKKLVVHGNKSDAIVLRSIYNLRALTSLNISHNFTATSLPEEMFKSLANLKYLEIAFISNLKELPNSLASLNALKHLFINCCFALESLPEEAVKGLTSLTQLSITYCKRLKCLPEGLQQLTNLSVRYCPTLAKRCEKGIGQDWYKIAHIPHLLITD

SEQ ID NO: 34 - DNA, 2940 bp, Solanum habrochaites
  source 1..2940: mol_type = genomic DNA
atggctgaagctttccttcaaattatgttagagaatctgacttgtttcatccaaggggaacttggattgattcttggttttaaggatgagttcgaaaagcttcaaagcacgtttactacaatccaagctgtggtacaagatgctcagttgaagcaattgaaggacaaggcaattgaaaattggttgcagaaactcaatggtgctgcatatgaagctgatgacatcttggacgaatgtaaaactgaggcaccaattatacagaagaagaataaatatgggtgttatcatccaaacgttatcactttccgtcgcaagattgggaaaaggatgaaaaagattatggagaaactagatgcaattgcagcggaacgaattaagtttcatttggatgaaaggactatagagagacaagttgctacacgccaaacaggttttgttttaaatgaaccacaagtttatggaagagacaaagataaggatgagatagtgaaaatcctgataaacaatgcccaaacactttcagtcctcccaatacttggtatggggggactaggaaagacgacccttgcccaaatggtcttcaatgatcagagagtaattgaacatttccatcccaaaatatggatttgtgtctcggaagattttaatgaaaagaggttgataaagaaaattgtagaatctattgaagaaaagtcacttggtgacatggacttggctccacttcaaaagaagcttcaggacttgctgaatggaaaaaaatatttgcttgtcttagatgatgtttggaatgaagatcaagataagtgggctaagttaagacaagtcttgaaggctggagcaagtggtgcttatgttctaaccactacccgtcttgaaaaggttggatcaatcatggggacattgcaaccatatgaattgtcaaatttgtctcaagaagattgttggttgttgttcatgcaatgtgcatttgggcaccaagaagaaatgaatcttaatctagtggctatcggaaaggtgattgtgaaaaaatgtggtggtgtgcctctagcagctaaaactcttggaggtattttgcgcttcaagagagaagaaagacagtgggaacatgtgagagatagtgagatttggaatttacctcaagatgaaagttctattctgcctgccctgagacttagttaccatcaccttccacttgatttgagacaatgcttttcatattgtgcagtattcccaaaggataccaaaatggaaaaggaaaatctaatctctctctggatggcacatggttttcttttatcaaaaggaaacttggagctagaggatgtaggtaatgaagtatggaatgaattatacttgaggtcttttttccaagagattgaagttcaatatgatcgaacttatttcaagatgcatgatctcattcatgatttggcaacatctctattttcagcaagcacatcaagcagcaatatccgagaaataaatgtagaaggttacctacatatgatgtcgattggtttcgcaaaagtggtgtcttcttactctcctcctcacttgcaaaagtttgtctcattgagggttcttaatctaagttccatgggacttaagcagttaccgtcctccattggagatctagtacatttaagatacttgaacctctctctcaataacatgcgtactcttccaaagcagttatgcaagcttcaaaatctgcagactcttaatgtagagtattgctggtcactttgttgttttccaaaagaaacaagtaaacttggtagtctccgaaatctcttacttgatggttgcgatggattggattctatgccaccaaggataggatctttgacatgccttaagactctaagtttatttgttattggcgagagaaaagattctctacttggtgaattacgaaacctgaatctgtatgggtcaattgaaatcacgcatcttgagagagtgaagaatgatagggatgcaaaagaagccaatttatctgcaaaagaaaatctgcattctttaagcatgagatgggaaggaccacatagatatgaatcagaagaagttgaagtgcttgaatccctcaaaccacactccaatgtgacttgtttaacaatcactggcttcagaggaatccgtctcccagagtggatgaatcactcagttttgaaaaatgttgtctctattgcaattagaggttgtgaaaactgctcatgcttaccaccgtttggtgatctgccttgtctagaaagtctagagttacggagtgggtctgcggaagtggagtatgttgaagattctggattcccaacaagaagaaggtttccatctatgagaaaacttactatagaaaattttgataatctgaaaggattgctgaaagaggcaggagaagagcaattccccgtgcttgaagagttgacaattagatgttgtcctgtgtttgttattccgaccctttcttctgtcaagaaattggtagttcatgggaacaagtcagatgcaatagttttgaggtccatatataatcttagggctcttacttccctcaacattagccataacttcacagctacttcgctcccagaagagatgttcaaaagccttgcaaatctcaaatacttggaaatcgctttcatctccaatctcaaagagctgccaaacagcctggctagtctcaatgctttgaagcatctgtttattaattgttgttttgcactagagagtctccccgaggaagcggtgaaaggtttaacttcactcacacagttatccataacatactgtaagaggctaaaatgtttaccagagggattgcagcaactaacaaatttatcagttaggtattgtccaacactggccaagcgatgtgagaagggaataggacaagactggtacaaaattgctcacattcctcatctgctgattactgattag

SEQ ID NO: 35 - DNA, 301 bp, Solanum lycopersicum
  source 1..301: mol_type = unassigned DNA
  misc_feature 151: allele = susceptible : A; resistant : G
gtgaaataatcatagaaaaaatctggttttggttggtaggttgacctctagtcatgattggtactaacagtttattctgagatgtctgtgacactttagatcttgggctagcttgaagcgttgtgtggaaatccttgagcttgtcagkgtrtgtgtgtattactataaaatgtatgcagagttgagtaagtaaagaaattttgcaggctccagttttgagagaatacccggcaagcttaacattgacagcatattcttacctttttggcgttataccaatgatagggacgtcattccttgt

SEQ ID NO: 36 - DNA, 301 bp, Solanum lycopersicum
  source 1..301: mol_type = unassigned DNA
  misc_feature 151: allele = Susceptible : C; resistant : A
ccccagccattggtactccaaagtgaagctcgtacattgcaggaatatttggagccaaggcagcaacctgctcagaaatttagctaaacataagagcagcaccaatataacagacagtgatctgaataagaactaatttccttcctgctamaaatataaaaacacctaactaagtgatagaggaggcaaatgcttaattgattccatgatatgttatactttcaacatacttgtgtttcatgactcatcactgacatctaagtcttagtgccacatcttttatgtttttttttaacaaacc

SEQ ID NO: 37 - DNA, 301 bp, Solanum lycopersicum
  source 1..301: mol_type = unassigned DNA
  misc_feature 151: allele = Susceptible : G; resistant : A
ggcacgcatgttgacatcaaagaagacagtattgtggtttgcataacggtcagaaggatcaattccctcaaaacgttgaggaaactgaacataacagatcctatctccaccgcggtccatcataaagcacattccctccctgattgcctcrgagttgtagatgtagtgatcacaatcaaggttgaggataaaggggccattagacatgatggcagaagctctgaccaaggcattcatggcaccagcctttttgttatgatcatagccaggacgcttytcacgagagacatacaccagcaag

SEQ ID NO: 38 - DNA, 301 bp, Solanum lycopersicum
  source 1..301: mol_type = unassigned DNA
  misc_feature 151: allele = Susceptible : G; resistant : A
tgtcatgtctctgagaagtgaacaagcagagtctacctcattgactatacataatgcttgtatcacccgagcaaaagtgaatacagaaggagaaatacctttccccaacatttcataaaatacatttggagcaactttgggacaatttcyrgctaacagaatatccaacgcttgattgtatgacttgaaagtgggttcacaagagaaagtattccacatatccaaaagtattctagttgcttgccctggtaaaccagctcttccataatgcctcattatcataataaagagggactctttg

SEQ ID NO: 39 - DNA, 121 bp, Solanum lycopersicum
  source 1..121: mol_type = unassigned DNA
  misc_feature 61: allele = Susceptible : G; resistant : C
atcaaaacacattctttaattctctagaaaataatatgaaggtgggatgtggtggagagtsgggagtgaggtttcgttgagctcttgcaatgaggatcttgtgtttcagtatggttcttga

SEQ ID NO: 40 - DNA, 301 bp, Solanum lycopersicum
  source 1..301: mol_type = unassigned DNA
  misc_feature 151: allele = Susceptible : A; resistant : G
attagacatgggaaacagatccattcacacctcatcagatctggtttttacaaaaatgcttttgtatcagctgctcttgtggatatgtatagtagctgtggcaggttagacattgctcttcaggtattccagagttcagccgagagatctrttgcagcttggaattcaatgatctctgcatatggtttccacagcaatggtcagaaagcaattgacatctttcatgagatgattaattcgggacttactccgtctaaagtcacatttatcaacttattaacagcttgcagccacacagggc

SEQ ID NO: 41 - DNA, 301 bp, Solanum lycopersicum
  source 1..301: mol_type = unassigned DNA
  misc_feature 151: allele = Susceptible : A; resistant : G
tatgttgaattttggtgctcctgccacaggggagagccctccttctcaaggtgatgcctcaagtgactcgtctgaggaaaatggcgacagtcctttccaacatgaatcaggaccttatggtaacgctggccaaccaatgcatggtatgtcratgtatgcaaatatgggctggccaaaatcgctgtgaaaacactttttctgtgtcgacatgaagttgccaagggtwttccccttgccaacgctaacagggcagagtataggcgcgtttcttgatttctcaacttgagcttattttccttat

SEQ ID NO: 42 - DNA, 101 bp, Solanum lycopersicum
  source 1..101: mol_type = unassigned DNA
  misc_feature 51: allele = Susceptible: A; resistant: G
ttccgaaatgaggacgatccatcagcttcttcagctgagagcccctggtcracataccagaattctgtttttctaaaactgtccaaaatctcctgtaaaga

SEQ ID NO: 43 - DNA, 71 bp, Solanum lycopersicum
  source 1..71: mol_type = unassigned DNA
  misc_feature 36: allele = Susceptible: T; resistant: C
gatttgaatgccttgccacagccagaggatgacgaygagatttttggacaacaattagaagatgaaccaca

SEQ ID NO: 44 - DNA, 121 bp, Solanum lycopersicum
  source 1..121: mol_type = unassigned DNA
  misc_feature 61: allele = Susceptible: A; resistant: G
agagaatgatatcactgccttagtttctcaattaaaagttgtgcaaaaacaaaacacacarctagatgaagaaaacagagcattcgcctcaaagcttcagacaaaagaagttgagaacaac

SEQ ID NO: 45 - DNA, 71 bp, Solanum lycopersicum
  source 1..71: mol_type = unassigned DNA
  misc_feature 36: allele = Susceptible: T; resistant: G
tacaataccttctggcatccctttccgcaaaacgakagatctttagtatcaaaaccgagagcactgtcacc

SEQ ID NO: 46 - DNA, 401 bp, Solanum lycopersicum
  source 1..401: mol_type = unassigned DNA
  misc_feature 201: allele = Susceptible: A; resistant: G
gttgggaggcagaagggtataagattggacactgaaatttttgctacttaccgtaagctcatcaacatcgactgttagaaacatcagagaaagatatttctcagacagctcacagtagaatggagagatcattctacatggaccacaccatgaagcactgaaatttgcaataacctgccaaaatttcacttgcgtaagaarggtccaaaatgccaaagccagcaaaaatgtaaaattttaagcaagcaaaaggcgagggatccatatggggaaactgctgcaccattctgctattatccttctcttcgcatgtgtgcaaagcattagagttaaagtgccttgagtattgttttttaacgtcttttgaattttagtgcagctgggctagaggattaagttac

SEQ ID NO: 47 - DNA, 401 bp, Solanum lycopersicum
  source 1..401: mol_type = unassigned DNA
  misc_feature 201: allele = Susceptible: A; resistant: G
tactgtaccaatattcagtgcaaggtataggtgtccattcatgagggctgtcacccatgaacaccgaaaatgctgctcacagtacttgctgagagcttcagacgaaccatccatttgcagataatatccagtgctcaagataggggtgtttgttggagccggtgaattttttccagaagagagtatatgtttgggatcacrgtctgtgacaatgacctcgggatgtccatgtagcctaataatttcagaagcaaatgaagaagccatagtttgagctgtgaaggtagaagaaaaggcaatgattgattgcccatatttgagttaccagtcaaccaacgtcaagattgttaccttcccttcgaccccaaaaggcaagtaatgcattggtacctccttaaaca

SEQ ID NO: 48 - DNA, 401 bp, Solanum lycopersicum
  source 1..401: mol_type = unassigned DNA
  misc_feature 201: allele = Susceptible: G; resistant: A
tactatcgaagggtaaaaataaaaaacaagttgttcaaatatccattagaatgaagaaaagaaaagcaagttaggggctcttggaccgggtggtatcaatgtctaagcaatggttctccagaggcgtcacaccattccaagccttattcagcatggagaaaaatccctgaaatctaatggctcaagtctagaaatagcctrgtcgatagacacacaagtaaaaattacctttaccgtcttctacatcattagcattaggcgttctctgaccactcccttcctcttctccttttgcaaatgacgacagaacatctcgacttcgagggaagttttgggtgggattggagtaaaatcgtgtattcactccccatactcagcctccaagtcctatttaacctgtg

SEQ ID NO: 49 - DNA, 401 bp, Solanum lycopersicum
  source 1..401: mol_type = unassigned DNA
  misc_feature 201: allele = Susceptible: G; resistant: A
aagtaggaatacggtccatggtctgtgttcatggattgagacataacttacccaatcacttaaatgaacaacagatgaccagcgcggagcattgttcaatctattgtccgtatgtttgatcataactgaggttagcagttagttacgggaaatttacggattggatcaacttaaaattgtcataaattttagcacaaaatraattaggttctccataacccatgatatgatagataatttaatattctctccatatccactgagtttgctaaaatttgacctccgagtgaaaagttatgcctattttagtgaaggcctattgagtagaccctaacgacagaccgtctattgaacaacggcccatcagtccaggtcgtcgatttccgcgacaacaattagct

SEQ ID NO: 50 - DNA, 401 bp, Solanum lycopersicum
  source 1..401: mol_type = unassigned DNA
  misc_feature 201: allele = Susceptible: C; resistant: A
aacaacccaatattcttaggaaatgatccttaaaatggtgcatcttgcttagacaaccaatgtaagggctacttgactagagagagaagtctctgggatgattgaagctaccacaacatttggtttagctcccctccatgtgtctattgatgatttgaccgctagggtcacaacttgtgagagcaagaaaggggagaaatmtgaggctttgtcttttaaagacaaggtagcaaagcagaagaaggacatagactatccaaagtctactgacttcacttcattactagaggctgctaatgatttagaaacaattgatactttagagattacttcgcctaccaccagagaggtacatagggaggatgctagaattgatgtattagatgctgaaactgatgagg

SEQ ID NO: 51 - DNA, 401 bp, Solanum lycopersicum
  source 1..401: mol_type = genomic DNA
  misc_feature 201: allele = Susceptible: G; resistant: A
cattgcgaaattgcatttgttctcttaatcaaagtcttgtaactttgggtgatttagtgtgttctcggtgaaccgatcggcaactcaccgactacacatttatgtcgccgacttgatttttccccctcccctccgggctagtatacttgaactgtaggcgaattggggagccactcggcagttcaccaattggttttggcrattaccaggatcttctttttctccacttgttcagctcgtttttctcccttttttctagaagtgtcctgccttgcttattaactcatataccttaaaattaatgctttaacaatagtttgtagaataaaataggcatttaaggacactcaaactaccaaaaaatatccctaaatgagtaaaatctttgactcattatttat

SEQ ID NO: 52 - DNA, 401 bp, Solanum lycopersicum
  source 1..401: mol_type = unassigned DNA
  misc_feature 201: allele = Susceptible: G; resistant: A
gtaatagggtcataaaatacttggtgtgaatttaccaaacaccctaagcattggcagaaaacatatagtgtttgaacccctcaaagacgagacctaacaaatagaccatctgcgcatcaacggaccgtttgtggggtctcgtgggttgacacttagctttacatggagcctgactaacaaaccttgaagatccaacagatrgagcaagaagacaggccatcggttcaaccattggctgtcgttgcctttgttggtgaacacctcaaggaaatttttaaccaagatttgggctctttctctagggccccttgtgggtcaaaggtggggtcgtactaaacaattaaattccttaactatgtaatataggtttaggaatatctcatgcaggttccaataaaaaa

SEQ ID NO: 53 - DNA, 401 bp, Solanum lycopersicum
  source 1..401: mol_type = unassigned DNA
  misc_feature 201: allele = Susceptible: A; resistant: C
tgaaatgtttaaggtttagattggttggttcgctcacataggagggtaagtgtgggtgccagccccagcccggttttgggtcgtgacaattggacttccatctgtggtgattttaatacaccgatatgggtttttcccatgctattatcttcactttagtttcatgagtgcaagcatcccctagttgaaagagacctttcmacattgaaggccatttgatttaaggaaatgttacagccaacattctgtatgtatccataataataacatattttactctactcatgtcggatgctcttccttaatatatgcttgcgcacctattctttcacaagatccaacatattatgacatgtctggcttataagagtagtttgtgtgccccattttggtgttttgtt

SEQ ID NO: 54 - DNA, 401 bp, Solanum lycopersicum
  source 1..401: mol_type = unassigned DNA
  misc_feature 201: allele = Susceptible: G; resistant: C
gacttgactcttaacttttagtcaattgaattatggattcaaagttaattatctcatgtttatggatgattttaagatttttgagtgtaccttaaagtgtgggaataaactagaaaacataggtatgttgctagggtactagtacgaaatgaagtgggaaaagattgatgaattggtaaccctggcgcattaagaggtgcsggccgcaagtgggtaacatcagatactagtgtagggttttttggctggtatggagccccagattccaaacttcagcgcccctaatgaggcgctttggctggaacagcgccttagtcccttgcccagttgaattccaacttttcttgctcatttttcaactctaaacccctaatttcaacttgattctttacccaaacaca

SEQ ID NO: 55 - DNA, 401 bp, Solanum lycopersicum
  source 1..401: mol_type = unassigned DNA
  misc_feature 201: allele = Susceptible: G; resistant: A
tatgagagagaattttttaaaccatctaatccatgaaaaggcttcacagacattagatcagtacaaggaccgtcaatggggttcgtcaacctaaaactacagacagtgtgatatttttatttttttccattgttagctcgtcatcctaaaagtgttttgagtgtgccgttaagatatattggtactaaaaatttctccgcrggtactatgtctcccaagaggcaagtggtttatacaaaaaggggcaaattcaagttagttggcccttcttcttggttaattgatgaagacctagactagaagagggacccagcctacattccttcggacataaaaacaccaccaacaaaaccctaaactactaagagcaccattctaatgaggaccgcacactgttcggc

SEQ ID NO: 56 - DNA, 401 bp, Solanum lycopersicum
  source 1..401: mol_type = unassigned DNA
  misc_difference 201: allele = Susceptible: A; resistant: C
aaaatttaaccatcaaatcttcattaaacatagacgtatgggatctaattcacctttgagacctctttcctcaaaggttttcttcaaagtattttcaaaagttgagttttcttgtaggtgcattcatccgaatttggacctcccacagtggttcaaagttcaaaaatttgatgtcattggaggaatagggaaccccttagmgcatctaaggtcctattgttacaaactaatgggagttaggagtgataaagctatattgatgcaactttttggcagaagattgagcggagaagctttggaactgtttaaatctcacgagaccagacaatgagattgctggaatgcctcggctaatgaattcattgagcgagttgcttacaacgtagagatcgtccctgact

SEQ ID NO: 57 - DNA, 401 bp, Solanum lycopersicum
  source 1..401: mol_type = unassigned DNA
  misc_feature 201: allele = Susceptible: A; resistant: C
gaactatctaacttctttcatcctccttttatgcgatagagttcatctttataaaagtttctttctaactcgtgcttgcacgtatcttttagatcatgcctccatgaagatttgtctgagctagtcgttctaggagcaacgttaaggatcaagaggtgcccaatgcactagaagtgcgaccctaaggagaggtcacttctmggatcggataatgagacagattgcgaccaacaaagctatgaaataaagaaagaattgacaggagctagttgatacttctagcattcgtgagtacttaaggatgaatttttcaagcttcaataattcaagtgtgactgaggatccagaatattttataaaatagttgtagaaagtgattgaggtcatgcgtgttgcttatg

SEQ ID NO: 58 - DNA, 401 bp, Solanum lycopersicum
  source 1..401: mol_type = unassigned DNA
  misc_feature 201: allele = Susceptible: A; resistant: G
agttagtgagttctatagtctttggcaagagttttggagtataatatgatggtaaaagcttgtaaaaatggtttccgtacttacaataggagtttaaggggtccgggtacgactccccaaataccaccaaggggtccttgaggaggaccctaacactgaccaaagagatttcaaaacaacttggttgatggttgcaattcrtgaccagtaggttggtctacaccccgtaagtcaagtcatgcaccactcctacaaagctgatcctcgggtgcaaaccacaaaggtggttgacgtacagtaacttctcctacagtccgtaggtggaggggtgtaggtgtttccagttttaagtaatttattttagttaggtgtttggtaatcgattagttatacaattacgg

SEQ ID NO: 59 - DNA, 401 bp, Solanum lycopersicum
  source 1..401: mol_type = unassigned DNA
  misc_feature 201: allele = Susceptible: A; resistant: G
cattaagagactaaggtcgtggggtaacaatccatggaccaggaccacgacctgtgacctggcctaagatccgtggatctggcagtgggtcgacccttagaggattttctggacctttttgggaagggttgtaggttgaaatccacggaccaccacttacagtctgtgcgtcaccctagtggtaactatggattgcacatrtagcttctggaattaacatgcaacttttggatgttggttggtctttttaggataaaaggtgttacattatctcccccttggaaataatcttcctcgagtgaagactaaactagctgaatatgaaggaaaagagcttccaaccctaccactaattactagaaactgatttctgactgaaataagttccaaggacatgcaaa

SEQ ID NO: 60 - DNA, 300 bp, Solanum lycopersicum
  source 1..300: mol_type = other DNA
  misc_feature 200: allele = Susceptible: G; resistant:A
tgaacatgtattattataagaatccaattaaaaacaaaatgaaacacttgaaagatccgtacttcccaaaaaaaattcctccttctcatggccttacaacaaaatttataaattctctttgttagaatagaaatatgtatatatatattcctacttagaataggaagaagaataggaataggaatcctagttagaaaagracttcaatgtattgtctataaatagggtctcaatgtaacaatttagatacacaattcaataatatttttccaatatatattctcacatggtatcagagca